(19) United States
(12) Patent Application Publication
(10) Pub. No.: US 2024/0245761 A1
(43) Pub. Date: Jul. 25, 2024

(54) METHODS OF TREATING CELIAC DISEASE

(71) Applicant: Curemark, LLC, Rye Brook, NY (US)

(72) Inventors: Joan M. Fallon, White Plains, NY (US); Matthew F. Heil, Sherman, CT (US); James Szigethy, Montgomery, NY (US); James J. Fallon, Armonk, NY (US)

(21) Appl. No.: 18/583,069

(22) Filed: Feb. 21, 2024

Related U.S. Application Data
(63) Continuation of application No. 17/742,753, filed on May 12, 2022, now abandoned.
Application No. 17/742,753 is a continuation of application No. 16/422,462, filed on May 24, 2019, now Pat. No. 11,364,287.
Application No. 16/422,462 is a continuation of application No. 13/836,135, filed on Mar. 15, 2013, now Pat. No. 10,350,278.
(60) Provisional application No. 61/653,253, filed on May 30, 2012.

(51) Int. Cl. A61K 38/54; A61K 38/46; A61K 38/47; A61K 38/48

(52) U.S. Cl. CPC A61K 38/54; A61K 38/465; A61K 38/47; A61K 38/48

(57) ABSTRACT

Described herein are compositions which include digestive enzymes and which are formulated to reduce one or more symptoms of Celiac disease. Also described herein is a method for treating a subject with Celiac disease using digestive enzymes and their derivatives to alleviate the symptoms of Celiac disease. The method comprises administering to the individual an effective amount of digestive enzymes that are either naturally or recombinantly derived, or their derivatives, in an amount effective to reduce one or more symptoms of Celiac disease.

### CROSS-REFERENCE

This application is a continuation of U.S. application Ser. No. 17/742,753, filed May 12, 2022, which is a continuation of U.S. application Ser. No. 16/422,462, filed May 24, 2019, now U.S. Pat. No. 11,364,287, issued Jun. 21, 2022, which is a continuation of U.S. application Ser. No. 13/836,135, filed Mar. 15, 2013, now U.S. Pat. No. 10,350,278, issued Jul. 16, 2019, which claims the benefit of U.S. Provisional Application No. 61/653,253, filed May 30, 2012, which application is incorporated herein by reference in its entirety.

### RELATED APPLICATIONS

This application is related to the following patents and patents pending, each of which are fully incorporated herein by reference: U.S. application Ser. No. 09/707,395 filed Nov. 7, 2000, issued on Oct. 14, 2003 as U.S. Pat. No. 6,632,429 B1, entitled “Methods for Treating Pervasive Developmental Disorders”; U.S. application Ser. No. 11/555,697 filed Nov. 2, 2006, entitled “Methods for Treating and Diagnosing Parkinson's Disease and Related Dysautonomic Disorders”; U.S. application Ser. No. 11/533,818 filed on Sep. 21, 2006, entitled “Pharmaceutical Preparations for Attention Deficit Disorder, Attention Deficit Hyperactivity Disorder and Other Associated Disorders”; U.S. application Ser. No. 12/386,051 filed Apr. 13, 2009, entitled “Enzyme Delivery Systems and Methods of Preparation and Use”; U.S. application Ser. No. 12/493,147 filed Jun. 26, 2009, entitled “Methods and Compositions for the Treatment of Symptoms of Complex Regional Pain Syndrome”; International Application No. PCT/US09/49374 filed Jul. 1, 2009, entitled “Methods and Compositions for the Treatment of Symptoms of Neurological and Mental Health Disorders”; U.S. application Ser. No. 12/426,794 filed Apr. 20, 2009, issued on Dec. 27, 2011 as U.S. Pat. No. 8,084,025, entitled “A Method for the Treatment of the Symptoms of Drug and Alcohol Addiction”; U.S. application Ser. No. 12/493,122 filed Jun. 26, 2009, entitled “Methods and Compositions for the Treatment of Symptoms of Williams Syndrome”; U.S. application Ser. No. 11/232,180 filed Sep. 21, 2005, entitled “Combination Enzyme for Cystic Fibrosis”; U.S. Provisional Application No. 61/102,818 filed Oct. 3, 2008, entitled “Pharmaceutical Preparation for the Treatment of Symptoms of Prion Diseases and Method of Making Same”; U.S. Provisional Application No. 61/253,805 filed Oct. 21, 2009, entitled “Methods and Compositions for the Prevention and Treatment of Influenza”, and International Application No. PCT/US12/34489 filed Apr. 20, 2012, entitled “Compounds for the Treatment of Neuropsychiatric Disorders”.

### BACKGROUND OF THE INVENTION

Celiac disease, also known as sprue, non-tropical sprue, gluten intolerance or gluten-sensitive enteropathy, is a condition that damages the lining of the small intestine and prevents it from absorbing parts of food important for general health. The damage is due to a reaction to eating gluten, which is found in wheat, barley, rye, and possibly oats. Parts of these grains can be found in a whole host of processed foods.

Celiac disease is both a disease of malabsorption, as nutrients are not absorbed properly, and an abnormal immune reaction to gluten. The exact cause of Celiac disease is unknown. The lining of the intestines contains areas called villi, which maximize the intestine's ability to absorb nutrients. When people with Celiac disease eat foods or use products that contain gluten, their immune system reacts by damaging these villi.

Celiac disease has a strong genetic component associated with the Histocompatibility-Linked-Antigen (HLA) region of the human genome. Environmental factors may also play a role in triggering the disease. People who have a family member with Celiac disease are at greater risk for developing the disease. Sometimes the disease is triggered or becomes active for the first time after surgery, pregnancy, childbirth, viral infection or severe emotional stress. The disorder is most common in Caucasians and persons of European ancestry with women being typically affected more often than men.

No description in the Background Section should be taken as an admission that such disclosure constitutes prior art to the instant invention.

### SUMMARY OF THE INVENTION

The present disclosure provides a method for using digestive enzymes and their derivatives to alleviate the symptoms of Celiac disease and related disorders. The method comprises administering to the individual a digestive enzyme either naturally or recombinantly derived, or their derivatives, in an amount effective to reduce the symptoms of Celiac disease. Digestive enzymes generally comprise all proteases, amylases, and lipases, as well as other proteins secreted in a mammal that affect the digestive process either directly or indirectly.

The disorders that present symptoms potentially suitable for alleviation according to a method described herein include, but are not limited to, Celiac disease, celiac sprue, non-tropical sprue, gluten-sensitive enteropathy, irritable bowel syndrome and chronic fatigue.

Prior to the present application the use of digestive enzymes in the treatment of Celiac disease has not been elucidated. The use of compositions comprising high doses of digestive enzymes to address the lack of protein digestion experienced by those with Celiac disease is described herein. The application of these enzymes of the high protease classification as applied to individuals with Celiac disease represents a novel discovery for the use of digestive enzymes.

In one aspect, provided herein is a method for treating a subject exhibiting one or more symptoms of Celiac disease or a related disorder comprising, administering a therapeutically effective amount of a composition comprising digestive enzymes to the individual. In one embodiment, the disorders comprise Celiac disease, non-tropical sprue, and celiac sprue.

In another embodiment, the symptoms of Celiac disease which are potentially suited for alleviation are for example, gastrointestinal symptoms, and include, but are not limited to, irritable bowel syndrome, protein deficiency, iron-deficiency anemia caused by menstrual blood loss or protein deficiency, inflammatory bowel disease, diverticulitis, intestinal infections, stool abnormalities, diarrhea which may be protracted or intermittent, constipation, alternating constipation with diarrhea, abdominal pain, bloating, gas or indigestion, decreased appetite (may also be increased or unchanged), lactose intolerance, nausea and vomiting, stools that float, are foul smelling, bloody, or “fatty”, and/or unexplained weight loss.

In certain embodiments, the symptoms of celiac and related disorders which are potentially suited for alleviation include, but are not limited to: depression, anxiety, chronic headaches, migraines, fatigue, memory loss, dementia, mania, hypomania, malaise (which may be protracted), seizures, tingling or numbness especially in the extremities and the head, seizures, and irritable and fussy behavior in children.

In another embodiment, the symptoms of celiac and related disorders which are potentially suited for alleviation are those, for example, which represent metabolic, dermatological, immunological or hormonal symptoms and include, but are not limited to, bruising easily, hair loss, mouth ulcers, nosebleeds, missed menstrual periods, Vitamin B12 deficiency, vitamin deficiencies, mineral deficiencies, osteoporosis, delayed growth in children, itchy skin (dermatitis herpetiformis), muscle cramps, joint pain, short stature, poor weight gain, slowed growth, failure to thrive, low cholesterol, pancreatic cancer, lymphoma, gastric cancer, colon cancer and intestinal cancer.

In one embodiment, the digestive enzymes are manufactured using technology selected from the group consisting of enteric coating, lipid encapsulation, direct compression, dry granulation, wet granulation and any combination thereof. In another embodiment, the preparation is administered orally via a dosage formulation selected from the group consisting of pills, tablets, capsules, microcapsules, mini-capsules, time-released capsules, mini-tabs, sprinkles, and any combination thereof. In one embodiment, the total amount of protease ranges per dose from 10,000 to about 1,500,000 U.S.P. units.

Another aspect relates to the method for treating Celiac disease and related disorders in a subject comprising administering an effective amount of a composition comprising one or more digestive enzymes to the individual. In one embodiment the symptoms of Celiac disease and related disorders are selected from a group comprising gastrointestinal symptoms and include, but are not limited to, irritable bowel syndrome, protein deficiency, iron-deficiency anemia caused by menstrual blood loss or protein deficiency, inflammatory bowel disease, diverticulitis, intestinal infections, stool abnormalities, diarrhea which may be protracted or intermittent, constipation, alternating constipation with diarrhea, abdominal pain, bloating, gas or indigestion, decreased appetite (may also be increased or unchanged), lactose intolerance, nausea and vomiting, stools that float, are foul smelling, bloody, or “fatty”, and unexplained weight loss. In yet another embodiment the symptoms comprise those which are neurological or neuropsychiatric symptoms and include but are not limited to: depression, anxiety, chronic headaches, migraines, fatigue, memory loss, dementia, mania, hypomania, malaise (which may be protracted) seizures, tingling or numbness especially in the extremities and the head, seizures and irritable and fussy behavior in children. In another embodiment, the symptoms are metabolic, dermatological, immunological or hormonal and include, but are not limited to, bruising easily, hair loss, mouth ulcers, nosebleeds, missed menstrual periods, low cholesterol, Vitamin B12 deficiency, vitamin deficiencies, mineral deficiencies, osteoporosis, delayed growth in children, itchy skin (dermatitis herpetiformis), muscle cramps, joint pain, short stature, poor weight gain, slowed growth, failure to thrive, lymphoma, gastric cancer, colon cancer, intestinal cancer and pancreatic cancer.

Provided herein are compositions of digestive enzymes which are useful in the prevention or treatment of one or more symptoms of Celiac disease or a related disorder. Digestive enzymes generally comprise all proteases, amylases, and lipases, as well as other proteins secreted in a mammal that affect the digestive process either directly or indirectly. Treatment of Celiac disease or a related disorder encompasses stasis of one or more symptoms (i.e., they do not worsen), as well as reduction (partial or complete) of one or more symptoms. In one embodiment, one or more symptoms of such disorders are reduced in severity or duration by about 2%, about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100%. In another embodiment, one or more symptoms of such disorders are reduced in severity or duration by about 2-fold, about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 90-fold, about 95-fold, about 100-fold or more. Compositions may include not only one or more digestive enzymes, but also one or more pharmaceutically acceptable carriers, excipients, buffers, fillers, binders, stabilizers, surfactants, diluents, extracts, lubricants, fillers, flavorings, preservatives, colorants, diluents, and coating agents, such as vegetable oil, crystalline oils, taste maskers, etc.

Digestive enzymes to be used in a composition described herein include amylase, lipase, protease, and any combination thereof. In one embodiment, the composition comprising digestive enzyme comprises amylase, lipase and protease. In another embodiment, the composition comprises amylase and lipase. In another embodiment, the composition comprises amylase and protease. In another embodiment, the composition comprises lipase and protease. The enzymes and the amount of each enzyme present in such compositions may be empirically determined by a physician based upon the patient to be treated. In another embodiment, the digestive enzyme is further selected from the group consisting of chymotrypsin, trypsin, pancreatin, papain and any combination thereof. Digestive enzymes may be derived from a source selected from the group consisting of animal enzymes, plant enzymes, synthetic enzymes, recombinant enzymes and any combination thereof. In one embodiment the animal enzyme is derived from a mammal. In another embodiment, the mammal is a pig. In one embodiment, digestive enzymes are derived from a mammalian pancreas. In one embodiment the pancreas is a pig pancreas.

Compositions (preparations) comprising digestive enzymes may be manufactured using any appropriate technology including, but not limited to, enteric coating, lipid encapsulation, direct compression, dry granulation, wet granulation, and any combination thereof. A preparation may be an oral dosage formulation such as, for example, pills, tablets, capsules, microcapsules, mini-capsules, time released capsules, mini-tabs, sprinkles, and any combination thereof. In one embodiment digestive enzymes are provided as a pharmaceutical composition. In one embodiment the pharmaceutical composition is in the form of encapsulated sprinkles. In one embodiment the encapsulation is a lipid coating. In one embodiment the lipid coating is a soy lipid coating.

In one embodiment, the total amount of protease in a composition ranges from about 5,000 to about 1,500,000 U.S.P. units/dose. In another embodiment, the total amount of amylase in a composition ranges from about 1,000 to about 15,000,000 U.S.P. units/dose. In another embodiment, the total amount of lipase in a composition ranges from about 1,500 to about 282,000 U.S.P. units/dose.

In any of such methods, one or more of the formulations described herein may be administered to a subject. Provided herein is a pharmaceutical composition comprising digestive enzymes for use in the methods described herein, wherein the digestive enzymes comprise about 23,000 U.S.P. units/dose of lipase, about 144,000 U.S.P. units/dose of amylase and about 140,000 U.S.P. units/dose of protease. Provided herein is a pharmaceutical composition comprising digestive enzymes for use in the methods described herein, wherein the digestive enzymes comprise about 23,040 U.S.P. units/dose of lipase, about 144,000 U.S.P. units/dose of amylase and about 140,400 U.S.P. units/dose of protease. Provided herein is a pharmaceutical composition comprising digestive enzymes for use in the methods described herein, wherein the digestive enzymes comprise about 16,800 U.S.P. units/dose of lipase, about 70,000 U.S.P. units/dose of protease, and about 70,000 U.S.P. units/dose of amylase. Provided herein is a pharmaceutical composition comprising digestive enzymes for use in the methods described herein, wherein the digestive enzymes comprise about 16,800 U.S.P. units/dose of lipase, about 110,000 U.S.P. units/dose of protease, and about 70,000 U.S.P. units/dose of amylase.

A dose of a pharmaceutical composition described herein may be formulated for oral administered in an amount of, for example, about 3 teaspoons, about 2.75 teaspoons, about 2.5 teaspoons, about 2.25 teaspoons, about 2 teaspoons, about 1.75 teaspoons, about 1.5 teaspoons, about 1.25 teaspoons, about 1 teaspoon, about ½ teaspoon, about ¼ teaspoon, or about ⅛ teaspoon.

A dose of a composition described herein may be formulated for oral administered in an amount of, for example, about ½ tablet, about 1 tablet, about 1.5 tablets, about 2 tablets, about 2.5 tablets, about 3 tablets, about 3.5 tablets, or about 4 tablets.

Compositions contemplated herein include, but are not limited to, pellets, capsules, caplets, beadlets, sprinkles, tablets, softgels or other carriers.

In one embodiment, a dose of a composition comprises about 8,400 U.S.P. units lipase, 35,000 U.S.P. units protease, and about 35,000 U.S.P. units amylase. In one embodiment, a dose of a composition comprises about 8,400 U.S.P. units lipase, 35,000 U.S.P. units protease, and about 35,000 U.S.P. units amylase. In one embodiment, a dose of a composition comprises about 16,800 U.S.P. units lipase, about 70,000 U.S.P. units protease, and about 70,000 U.S.P. units amylase. In one embodiment, a dose of a composition comprises about 33,600 U.S.P. units lipase, about 140,000 U.S.P. units protease, and about 140,000 U.S.P. units amylase. In one embodiment, a dose of a composition comprises about 50,400 U.S.P. units lipase, about 210,000 U.S.P. units protease, and about 210,000 U.S.P. units amylase. In one embodiment, a dose of a composition comprises about 67,200 U.S.P. units lipase, about 280,000 U.S.P. units protease, and about 280,000 U.S.P. units amylase. In one embodiment, a dose of a composition comprises about 84,000 U.S.P. units lipase, about 350,000 U.S.P. units protease, and about 350,000 U.S.P. units amylase. In one embodiment, a dose of a composition comprises about 100,800 U.S.P. units lipase, about 420,000 U.S.P. units protease, and about 420,000 U.S.P. units amylase. In one embodiment, a dose of a composition comprises about 117,600 U.S.P. units lipase, about 490,000 U.S.P. units protease, and about 490,000 U.S.P. units amylase. In one embodiment, a dose of a composition comprises about 134,400 U.S.P. units lipase, about 560,000 U.S.P. units protease, and about 560,000 U.S.P. units amylase. In one embodiment, a dose of a composition comprises about 151,200 U.S.P. units lipase, about 630,000 U.S.P. units protease, and about 630,000 U.S.P. units amylase. In one embodiment, a dose of a composition comprises about 168,000 U.S.P. units lipase, about 700,000 U.S.P. units protease, and about 700,000 U.S.P. units amylase. In one embodiment, a dose of a composition comprises about 184,800 U.S.P. units lipase, about 770,000 U.S.P. units protease, and about 770,000 U.S.P. units amylase. In one embodiment, a dose of a composition comprises about 201,600 U.S.P. units lipase, about 840,000 U.S.P. units protease, and about 840,000 U.S.P. units amylase. In one embodiment, a dose of a composition comprises about 218,400 U.S.P. units lipase, about 910,000 U.S.P. units protease, and about 910,000 U.S.P. units amylase. In one embodiment, a dose of a composition comprises about 235,200 U.S.P. units lipase, about 980,000 U.S.P. units protease, and about 980,000 U.S.P. units amylase. In one embodiment, a dose of a composition comprises about 252,000 U.S.P. units lipase, about 1,050,000 U.S.P. units protease, and about 1,050,000 U.S.P. units amylase. Any of the doses may be optionally supplemented with additional protease. In one embodiment, a dose may be supplemented with about 0 U.S.P. units; about 20,000 U.S.P. units; about 40,000 U.S.P. units; about 60,000 U.S.P. units; about 80,000 U.S.P. units; about 100,000 U.S.P. units; about 120,000 U.S.P. units; about 140,000 U.S.P. units; or about 160,000 U.S.P. units protease.

In one embodiment, a dose of pharmaceutical composition comprises digestive enzymes, wherein the digestive enzymes comprise a dose as set forth in Table 1.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a graph illustrating increase in weight gain prior to and following administration of enzyme compositions to eight patients with Celiac disease.

### DETAILED DESCRIPTION OF THE INVENTION

The present inventors have identified compositions comprising digestive enzymes which are useful for prevention and/or treatment of Celiac disease (CD).

### Celiac Disease

Celiac disease affects people in all parts of the world. Originally thought to be a rare childhood syndrome, Celiac disease is now known to be a common genetic disorder. More than 2 million people in the United States have the disease, or about 1 in 133 people. Among people who have a first-degree relative such as a parent, sibling, or child diagnosed with Celiac disease, as many as 1 in 22 may have the disease.

Gluten (from Latin gluten “glue”) is a protein composite that appears in foods processed from wheat and related species, including barley and rye. It is utilized in the processing of food to give it elasticity, for example with dough, helping it to rise and keep its shape, and often giving the final product a chewy texture. It is used in products that range from foodstuffs to lipsticks and other cosmetics.

Gluten is the composite of a gliadin and a glutelin, which exist conjoined with starch, in the endosperm of various grass-related grains. The prolamin and glutelin from wheat—gliadin, which is alcohol soluble, and glutenin, which is only soluble in dilute acids or alkalis—compose about 80% of the protein contained in wheat seed.

Being insoluble in water, the gliadin and glutelin can be purified by washing away the associated starch. Worldwide, gluten is a source of protein, both in foods prepared directly from sources containing it, and as an additive to foods otherwise low in protein.

The seeds of most flowering plants have endosperms with stored protein to nourish embryonic plants during germination. True gluten, with gliadin and glutenin, is limited to certain members of the grass family. The stored proteins of maize and rice are sometimes called glutens, but their proteins differ from wheat gluten by lacking gliadin.

Gluten is extracted from flour by washing out the starch: starch is water-soluble while gluten is not, and gluten binds together strongly, while starch dissolved in cold water is mobile. If a saline solution is used instead of water, a purer protein is obtained, with certain harmless impurities going into solution with the starch. However, on an industrial scale, starch is the prime product, making cold water the favored solvent.

In home or restaurant cooking, a ball of wheat flour dough is kneaded under water until the starch dissolves out. In industrial production, a slurry of wheat flour is kneaded vigorously by machinery until the starch dissolves and the gluten condenses into a mass. This mass is collected by centrifugation, and then transported through several stages integrated in a continuous process. Approximately 65% of the water in the wet gluten is removed by means of a screw press; the remainder is sprayed through an atomizer nozzle into a drying chamber, where it remains at an elevated temperature a short time to evaporate the water without denaturing the gluten. The process yields a flour-like powder with 7% moisture content, which is air-cooled and pneumatically transported to a receiving vessel. In the final step, the collected gluten is sifted and milled to produce a uniform product.

Recognizing Celiac disease can be difficult because some of its symptoms are seen in other diseases. Celiac disease can be often misdiagnosed as irritable bowel syndrome, iron-deficiency anemia caused by menstrual blood loss, inflammatory bowel disease, diverticulitis, intestinal infections, and chronic fatigue syndrome. As a result, Celiac disease has long been under diagnosed or misdiagnosed.

Abnormalities with stools are the hallmark of the disease. Most individuals experience protracted diarrhea when they have Celiac disease. However, some individuals experience constipation, some have alternating constipation with diarrhea and still others experience very little problems with their stools.

Other gastrointestinal symptoms associated with Celiac disease include: abdominal pain, bloating, gas, or indigestion, decreased appetite (may also be increased or unchanged), lactose intolerance, nausea and vomiting, stools that float, are foul smelling, bloody, or “fatty”, and unexplained weight loss (although people can be overweight or of normal weight). Further symptoms of Celiac disease may include diarrhea which may be intermittent, an accompanying lactose intolerance, nausea, vomiting which may be intermittent or connected to the ingestion of food,

More symptoms may include easy bruising, depression, anxiety, chronic headaches, migraines, fatigue, malaise (which may be protracted), hair loss, mouth ulcers, seizures, muscle cramps, joint pains, tingling or numbness, especially in the extremities and the head, nosebleeds, and missed menstrual periods.

As the intestines in Celiac disease do not absorb many important vitamins, minerals and other parts of food, the following symptoms may arise over time: bruising easily, depression or anxiety, fatigue, delayed growth in children, hair loss, itchy skin (dermatitis herpetiformis), missed menstrual periods, mouth ulcers, muscle cramps and joint pain, nosebleeds, seizures, tingling or numbness in the hands or feet, unexplained short height and osteoporosis.

Children with Celiac disease may have defects in the tooth enamel and changes in tooth color, delayed puberty, diarrhea, constipation, fatty or foul-smelling stools, nausea, or vomiting, irritable and fussy behavior, poor weight gain, slowed growth and shorter than normal height for their age and depression.

Individuals with Celiac disease have higher than normal levels of certain autoantibodies, proteins that react against the body's own cells or tissues in their blood. Diagnosis of Celiac disease may include blood tests for high levels of anti-tissue transglutaminase antibodies (tTGA) or anti-endomysium antibodies (EMA). If test results are negative but Celiac disease is still suspected, additional blood tests may be needed. Also, an anti-gliadin antibody may be detected in the blood. The following indirect markers may be found in Celiac disease: low albumin in the blood, high levels of alkaline phosphatase, low cholesterol, abnormal complete blood count, low iron and decreased prothrombin time, as well as other clotting factors.

Dermatitis herpetiformis (DH) is an intensely itchy, blistering skin rash that affects 15 to 25 percent of people with Celiac disease. The rash usually occurs on the elbows, knees, and buttocks. Most people with DH have no digestive symptoms of Celiac disease. DH is diagnosed through blood tests and a skin biopsy. If the antibody tests are positive and the skin biopsy has the typical findings of DH, patients do not need to have an intestinal biopsy. Both the skin disease and the intestinal disease respond to a gluten-free diet and recur if gluten is added back into the diet. The rash symptoms can be controlled with immunosuppressants such as dapsone. Because dapsone does not treat the intestinal condition, people with DH must maintain a gluten-free diet.

Up to 30% of patients with Celiac disease also complain of concomitant chronic headaches or migraines. Systemic inflammatory responses to undigested proteins may lead to the advent of headaches and migraines.

There presently is no cure for Celiac disease, and multiple attempts to secure a pharmaceutical treatment of any kind have been unsuccessful. The only known treatment heretofore for Celiac disease is a gluten-free diet. A dietitian is generally employed to help teach the individual with Celiac disease how to employ the gluten free diet. Individuals with Celiac disease can learn from a dietitian how to read ingredient lists and identify foods that contain gluten in order to make informed decisions at the grocery store and when eating out.

Following the gluten free diet will stop symptoms, heal existing intestinal damage, and prevent further damage. Improvement begins within days of starting the diet. The small intestine usually heals in 3 to 6 months in children but may take several years in adults. A healed intestine means a person now has villi that can absorb nutrients from food into the bloodstream.

To stay well, people with Celiac disease must avoid gluten for the rest of their lives. Eating even a small amount of gluten can damage the small intestine. The damage will occur in anyone with the disease, including people without noticeable symptoms. Depending on a person's age at diagnosis, some problems will not improve, such as short stature and dental enamel defects.

Some people with Celiac disease show no improvement on the gluten-free diet. The most common reason for poor response to the diet is that small amounts of gluten are still being consumed. Hidden sources of gluten include additives such as modified food starch, preservatives, and stabilizers made with wheat. And because many corn and rice products are produced in factories that also manufacture wheat products, they can be contaminated with wheat gluten.

Rarely, the intestinal injury will continue despite a strictly gluten-free diet. People with this condition, known as refractory Celiac disease, have severely damaged intestines that cannot heal. Because their intestines are not absorbing enough nutrients, they may need to receive nutrients directly into their bloodstream through a vein, or intravenously. Researchers are evaluating drug treatments for refractory Celiac disease.

### Diagnosis

Americans are not routinely screened for Celiac disease. However, because Celiac disease is hereditary, family members of a person with the disease may wish to be tested. Four to 12 percent of an affected person's first-degree relatives will also have the disease.

Screening for Celiac disease generally refers to testing for the presence of auto-antibodies in the blood in people without symptoms. There are several tests that can be used to assist in diagnosis including, but not limited to, albumin levels (may be low), alkaline phosphatase levels (high level may be a sign of bone loss), clotting factor abnormalities, cholesterol (may be low), complete blood count (CBC—test for anemia), liver enzymes (transaminases), and prothrombin time. Blood tests can detect several special antibodies, called anti-tissue transglutaminase antibodies (tTGA) or anti-endomysium antibodies (EMA).

If the tests are positive, upper endoscopy is usually performed to sample a piece of tissue (biopsy) from the first part of the small intestine (duodenum). The biopsy may show a flattening of the villi in the parts of the intestine below the duodenum.

The level of symptoms may determine the order of the tests, but tests lose their usefulness if the patient is already taking a gluten-free diet. Intestinal damage begins to heal within weeks of gluten being removed from the diet, and antibody levels decline over months. For those who have already started on a gluten-free diet, it may be necessary to perform a re-challenge with some gluten-containing food in one meal a day over 2-6 weeks before repeating the tests.

High-risk symptoms include, for example, weight loss, anemia (hemoglobin less than 120 g/l in females or less than 130 g/l in males), or diarrhea (more than three loose stools per day).

Serological blood tests may be used to make a diagnosis of Celiac disease. IgA anti-endomysial antibodies can detect Celiac disease with a sensitivity and specificity of 90% and 99% according to a systematic review. The systematic review estimates that the prevalence of Celiac disease in primary care patients with gastrointestinal symptoms to be about 3%. Serology for anti-tTG antibodies was initially reported to have a high sensitivity (99%) and specificity (>90%) for identifying Celiac disease; however, the systematic review found the two tests were similar. Modern anti-tTG assays rely on a human recombinant protein as an antigen. tTG testing should be done first as it is an easier test to perform. An equivocal result on tTG testing should be followed by antibodies to endomysium.

If the tests are positive, upper endoscopy is usually performed to sample a piece of tissue (biopsy) from the first part of the small intestine (duodenum). The biopsy may show a flattening of the villi in the parts of the intestine below the duodenum. Genetic testing of the blood for specific markers is also available to help determine who may be at risk for Celiac disease. A follow-up biopsy or blood test may be ordered several months after the diagnosis and treatment. These tests evaluate your response to treatment. Normal results mean that you have responded to treatment, which confirms the diagnosis. However, this does not mean that the disease has been cured.

If blood tests and symptoms suggest Celiac disease, a biopsy of the small intestine should be performed to confirm the diagnosis. During the biopsy, the doctor removes tiny pieces of tissue from the small intestine to check for damage to the villi. To obtain the tissue sample, an endoscope is used which is inserted through the patient's mouth and stomach into the small intestine. The doctor then takes the samples using instruments passed through the endoscope.

### Compositions and Formulations

The invention also relates to a specific blend of enzymes, with or without coating, with or without other components as described above whereby enzyme administration occurs in individuals with Celiac disease and related disorders, including but not limited to: celiac sprue, non-tropical sprue, gluten-sensitive enteropathy and irritable bowel syndrome.

Digestive enzymes are produced by the salivary glands, glands in the stomach, the pancreas and glands in the small intestines. Digestive enzymes produced by the pancreas are secreted into the duodenum, or upper segment of the small intestine, raising the pH to around 5 or 6, and they assist in the digestion of food components, including carbohydrates, lipids, proteins and nucleic acids.

Pancreatic enzymes administered to humans are commonly of porcine origin. Manufacturers of enzyme preparations for these individuals have used enteric coatings for targeted complete delivery in the distal portion of the small intestine, where lipase activity is important.

Digestive enzymes generally comprise all proteases, amylases and lipases, as well as other proteins secreted in a mammal which affect the digestive process either directly or indirectly.

Digestive enzymes to be used in the compositions and methods described herein include, for example, pancreatic enzymes. There are two types of pancreatic enzymes which have U.S.P. designations: pancreatin and pancrealipase. Pancreatin is a substance containing enzymes, principally amylase, lipase, and protease, obtained from the pancreas of the hog Sus scrofa Linne var. domesticus Gray (Fam. Suidae) or of the ox Bos taurus Linne (Fam. Bocidae). Pancreatin contains, in each mg, not less than 25 U.S.P. units of amylase activity, not less than 2 U.S.P. units of lipase activity, and not less than 25 U.S.P. of protease activity. More information on Pancreatin is provided in Example 1 below. In contrast, pancrealipase U.S.P. refers to a cream-colored, amorphous powder, having a faint, characteristic (meaty), but not offensive odor, which contains lipase in an amount of not less than 24 U.S.P. Units/mg; protease in an amount of not less than 100 U.S.P. Units/mg; and amylase in an amount of not less than 100 U.S.P. Units/mg; with not more than 5% fat and not more than 5% loss on drying.

In another aspect, this disclosure relates to a pharmaceutical composition comprising a therapeutically effective amount of an enzyme preparation, which comprises a core amount of pancreatic or digestive enzymes effective for treating a subject susceptible to treatment by the enzymes, specifically those suffering with Celiac disease or a related disorder.

In another aspect, this disclosure relates to a pharmaceutical composition comprising a therapeutically effective amount of an enzyme preparation, which comprises a core amount of pancreatic or digestive enzymes effective for treating a subject susceptible to treatment by the enzymes, specifically those suffering with Celiac disease and related disorders.

Compositions comprising an effective amount of the compound may be administered via any conventional route including but not limited to oral, parenteral, intramuscular, intravenous, transmucosal, transdermal, via suppository or other method. Further the oral administration can be in the form of pellets, capsules, caplets, beadlets, sprinkles, tablets, softgels or other carrier. The enzymes may be coated or uncoated.

The composition of the dosage form may include other components, generally utilized in pharmaceutical preparations including but not limited to binders, extracts, lubricants, fillers, flavorings, preservatives, colorants, taste maskers, diluents, and coating agents, such as vegetable oil, crystalline oils, and other coating methodologies.

In one embodiment, the digestive enzymes comprise amylase, lipase, protease, or a combination thereof. In another embodiment, the digestive enzyme is further selected from the group consisting of chymotrypsin, trypsin, pancreatin, papain and any combination thereof. Digestive enzymes may be derived from a source selected from the group consisting of animal enzymes, plant enzymes, synthetic enzymes, recombinant enzymes and any combination thereof. In one embodiment the digestive enzymes are pancreatic digestive enzymes. In one embodiment, the animal enzyme is derived from a mammal. In one embodiment the mammal is a pig. In one embodiment, digestive enzymes are derived from a mammalian pancreas. In one embodiment the pancreas is a pig pancreas.

In yet another embodiment of the present disclosure, digestive enzymes comprise proteases, amylases, and lipases, as well as other proteins secreted in a mammal that affect the digestive process either directly or indirectly. In one embodiment, the digestive or pancreatic enzyme composition comprises one or more of the following: amylases, proteases, cellulase, papaya, bromelain, lipases, chymotrypsin, trypsin, and elastase.

In one embodiment of the present disclosure, digestive enzymes comprise proteases, amylases and lipases, as well as other proteins secreted in a mammal that affect the digestive process either directly or indirectly. In one aspect, digestive enzymes present in the composition include an amylase, a protease, or a lipase. In another aspect, digestive enzymes present in the composition include two or more of: an amylase, a protease, and a lipase. In one aspect, digestive enzymes present in the composition include an amylase, a protease, and a lipase. In another aspect, a composition may further contain one or more of cellulase, papaya, bromelain, chymotrypsin, and trypsin. In one embodiment, the digestive or pancreatic enzyme composition comprises one or more of the following: amylases, proteases, cellulase, papaya, bromelain, lipases, chymotrypsin, and trypsin.

Compositions may contain an amount of protease from about 5,000; about 7,500; about 10,000; about 15,000; about 20,000; about 25,000; about 30,000; about 35,000; about 40,000; about 50,000; about 55,000; about 65,000; about 70,000; about 75,000; about 90,000; about 95,000; about 100,000; about 110,000; about 115,000; about 130,000; about 140,000; about 140,400; about 150,000; about 155,000; about 160,000; about 170,000; about 175,000; about 180,000; about 190,000; about 195,000; about 200,000; about 210,000; about 220,000; about 230,000; about 240,000; about 250,000; about 280,000; about 290,000; about 300,000; about 310,000; about 320,000; about 330,000; about 340,000; about 350,000; about 360,000; about 370,000; about 380,000; about 390,000; about 400,000; about 410,000; about 420,000; about 430,000; about 440,000; about 450,000; about 465,000; about 470,000; about 480,000; about 490,000; about 500,000; about 510,000; about 520,000; about 530,000; about 540,000; about 550,000; about 560,000; about 570,000; about 580,000; about 590,000; about 600,000; about 610,000; about 620,000; about 630,000; about 640,000; about 650,000; about 660,000; about 670,000; about 680,000; about 690,000; about 700,000; about 710,000; about 720,000; about 730,000; about 740,000; about 750,000; about 760,000; about 770,000; about 780,000; about 790,000; about 800,000; about 810,000; about 820,000; about 830,000; about 840,000; about 850,000; about 860,000; about 870,000; about 880,000; about 890,000; about 900,000; about 910,000; about 920,000; about 930,000; about 940,000; about 950,000; about 960,000; about 970,000; about 980,000; about 990,000; about 1,000,000; about 1,010,000; about 1,020,000; about 1,020,000; about 1,030,000; about 1,040,000; about 1,050,000; about 1,060,000; about 1,070,000; about 1,080,000; about 1,090,000; about 1,100,000; about 1,100,000; about 1,120,000; about 1,130,000; about 1,140,000; about 1,150,000; about 1,170,000; about 1,190,000; about 1,200,000; about 1,210,000; about 1,250,000; about 1,300,000; about 1,350,000; about 1,400,000; about 1,450,000; or about 1,500,000; about 1,200,000; about 1,250,000; about 1,300,000; about 1,350,000; about 1,400,000; about 1,450,000; or about 1,500,000 U.S.P. units/dose along with all values in between per dose.

Compositions may contain an amount of amylase from about 1,000 to about 15,000,000; from about 5,000 to about 1,000,000; from about 15,000 to about 750,000; from about 50,000 to about 500,000; from about 75,000 to about 250,000; from about 95,000 to about 200,000; or from about 100,000 to about 150,000 U.S.P. units/dose. Compositions may contain an amount of amylase including, but not limited to, about 1,000; about 3,000; about 5,000; about 7,500; about 10,000; about 15,000; about 20,000; about 25,000; about 30,000; about 35,000; about 40,000; about 50,000; about 65,000; about 70,000; about 75,000; about 100,000; about 140,000; about 144,000; about 210,000; about 280,000; about 350,000; about 420,000; about 490,000; about 500,000; about 560,000; about 630,000; about 700,000; about 770,000; about 840,000; about 910,000; about 980,000; about 1,000,000; about 1,050,000; about 2,000,000; about 3,000,000; about 4,000,000; about 5,000,000; about 6,000,000; about 7,000,000; about 8,000,000; about 9,000,000; about 10,000,000; about 11,000,000; about 12,000,000; about 13,000,000; about 14,000,000; and about 15,000,000 U.S.P. units/dose, along with all values in-between per dose

Compositions may contain an amount of lipase from about 1,500 to about 282,000; from about 5,000 to about 200,000; from about 5,000 to about 150,000; from about 75,000 to about 100,000; from about 10,000 to about 75,000; from about 15,000 to about 50,000; or from about 20,000 to about 40,000 U.S.P. units/dose. Compositions may contain an amount of lipase including, but not limited to, about 1,500; about 1,880; about 2,000; about 3,000; about 5,000; about 7,500; about 8,400; about 10,000; about 15,000; about 16,800; about 20,000; about 23,000; about 23,040; about 25,000; about 30,000; about 33,600; about 40,000; about 50,000; about 50,400; about 65,000; about 67,200; about 75,000; about 84,000; about 100,000; about 100,800; about 117,600; about 125,000; about 134,400; about 150,000; about 151,200; about 168,000; about 184,800; about 200,000; about 201,600; about 218,400; about 235,200; about 250,000; about 252,000; and about 282,000 U.S.P. units/dose along with all values in-between per dose.

In another embodiment, the digestive enzyme composition is comprised of protease, lipase, and amylase where the activity of protease is between about 5,000 to about 1,500,000 U.S.P. units/dose, or between about 10,000 to about 1,500,000 U.S.P. units/dose including, but not limited to, about 5,000; about 7,500; about 10,000; about 15,000; about 20,000; about 25,000; about 30,000; about 35,000; about 40,000; about 50,000; about 55,000; about 65,000; about 70,000; about 75,000; about 90,000; about 95,000; about 100,000; about 110,000; about 115,000; about 130,000; about 140,000; about 140,400; about 150,000; about 155,000; about 160,000; about 170,000; about 175,000; about 180,000; about 190,000; about 195,000; about 200,000; about 210,000; about 220,000; about 230,000; about 240,000; about 250,000; about 280,000; about 290,000; about 300,000; about 310,000; about 320,000; about 330,000; about 340,000; about 350,000; about 360,000; about 370,000; about 380,000; about 390,000; about 400,000; about 410,000; about 420,000; about 430,000; about 440,000; about 450,000; about 465,000; about 470,000; about 480,000; about 490,000; about 500,000; about 510,000; about 520,000; about 530,000; about 540,000; about 550,000; about 560,000; about 570,000; about 580,000; about 590,000; about 600,000; about 610,000; about 620,000; about 630,000; about 640,000; about 650,000; about 660,000; about 670,000; about 680,000; about 690,000; about 700,000; about 710,000; about 720,000; about 730,000; about 740,000; about 750,000; about 760,000; about 770,000; about 780,000; about 790,000; about 800,000; about 810,000; about 820,000; about 830,000; about 840,000; about 850,000; about 860,000; about 870,000; about 880,000; about 890,000; about 900,000; about 910,000; about 920,000; about 930,000; about 940,000; about 950,000; about 960,000; about 970,000; about 980,000; about 990,000; about 1,000,000; about 1,010,000; about 1,020,000; about 1,020,000; about 1,030,000; about 1,040,000; about 1,050,000; about 1,060,000; about 1,070,000; about 1,080,000; about 1,090,000; about 1,100,000; about 1,100,000; about 1,120,000; about 1,130,000; about 1,140,000; about 1,150,000; about 1,170,000; about 1,190,000; about 1,200,000; about 1,210,000; about 1,250,000; about 1,300,000; about 1,350,000; about 1,400,000; about 1,450,000; or about 1,500,000; about 1,200,000; about 1,250,000; about 1,300,000; about 1,350,000; about 1,400,000; about 1,450,000; or about 1,500,000 U.S.P. units/dose along with all values in between per dose and where the ratio of protease to lipase is such that the amount of lipase is never more than 0.188 times the amount of protease; and further wherein the ratio of protease activity to amylase activity is between 1:0.1 and 1:10.

In some embodiments, the digestive enzyme composition comprises at least one protease and at least one lipase, wherein the ratio of total proteases to total lipases (in U.S.P. units) ranges from about 5.371:1 to about 20:1 including 5.371:1, 6:1, 7:1, 8:1, 9:1, 10:1, 11:1, 12:1, 13:1, 14:1, 15:1, 16:1, 17:1, 18:1, 19:1 and 20:1, along with all values in-between. In some embodiments, the ratio of proteases to lipases ranges from about 5.371:1 to about 10:1 including 5.371:1, 6:1, 7:1, 8:1, 9:1, and 10:1, along with all values in-between.

In yet another embodiment, the digestive enzyme composition comprises at least one protease and at least one lipase, wherein the ratio of total proteases to total lipases (in U.S.P. units/dose) ranges from about 5.371:1 to about 20:1 including 5.371:1, 6:1, 7.1, 8.1, 9:1, 10:1, 11:1, 12:1, 13:1, 14:1, 15:1, 16:1, 17:1, 18:1, 19:1, and 20:1, along with all values in-between. In another embodiment, the digestive enzyme composition comprises at least one protease and at least one lipase, wherein the ratio of total proteases to total lipases (in U.S.P. units/dose) ranges from about 1:1 to about 20:1. In yet another embodiment, the ratio of proteases to lipases ranges from about 4:1 to about 10:1. In one embodiment, the ratio of proteases to lipases ranges from about 5.371:1 to about 10:1 including 5.371:1, 6:1, 7:1, 8:1, 9:1, and 10:1 along with all values in-between. In one embodiment, the digestive enzyme composition comprises at least one protease and at least one amylase, wherein the ratio of total proteases to total amylases (in U.S.P. units/dose) ranges from about 1:0.1 to about 1:10 including 1:0.25, 1:0.5, 1:0.75, 1:1, 1:1.25, 1:1.5, 1:1.75: 1:2, 1:1.25, 1:1.5, 1:1.75, 1:1.2, 1:1.25, 1:1.5, 1:1.75, 1:1.2, 1:1.25, 1:1.5, 1:1.75, 1:1.2: 1:1.5, 1:2, 1:3, 1:4, 1:5, 1:6, 1:7, 1:8, 1.9 and 1:10 along with all values in-between.

In another embodiment, the digestive enzyme composition is comprised of protease, lipase, and amylase where the activities are: protease between about 5,000 to about 1,500,000 U.S.P. units/dose, or between about 10,000 to about 1,500,000 U.S.P. units/dose including about 5,000; about 7,500; about 10,000; about 15,000; about 20,000; about 25,000; about 30,000; about 35,000; about 40,000; about 50,000; about 55,000; about 65,000; about 70,000; about 75,000; about 90,000; about 95,000; about 100,000; about 110,000; about 115,000; about 130,000; about 140,000; about 140,400; about 150,000; about 155,000; about 160,000; about 170,000; about 175,000; about 180,000; about 190,000; about 195,000; about 200,000; about 210,000; about 220,000; about 230,000; about 240,000; about 250,000; about 280,000; about 290,000; about 300,000; about 310,000; about 320,000; about 330,000; about 340,000; about 350,000; about 360,000; about 370,000; about 380,000; about 390,000; about 400,000; about 410,000; about 420,000; about 430,000; about 440,000; about 450,000; about 465,000; about 470,000; about 480,000; about 490,000; about 500,000; about 510,000; about 520,000; about 530,000; about 540,000; about 550,000; about 560,000; about 570,000; about 580,000; about 590,000; about 600,000; about 610,000; about 620,000; about 630,000; about 640,000; about 650,000; about 660,000; about 670,000; about 680,000; about 690,000; about 700,000; about 710,000; about 720,000; about 730,000; about 740,000; about 750,000; about 760,000; about 770,000; about 780,000; about 790,000; about 800,000; about 810,000; about 820,000; about 830,000; about 840,000; about 850,000; about 860,000; about 870,000; about 880,000; about 890,000; about 900,000; about 910,000; about 920,000; about 930,000; about 940,000; about 950,000; about 960,000; about 970,000; about 980,000; about 990,000; about 1,000,000; about 1,010,000; about 1,020,000; about 1,020,000; about 1,030,000; about 1,040,000; about 1,050,000; about 1,060,000; about 1,070,000; about 1,080,000; about 1,090,000; about 1,100,000; about 1,100,000; about 1,120,000; about 1,130,000; about 1,140,000; about 1,150,000; about 1,170,000; about 1,190,000; about 1,200,000; about 1,210,000; about 1,250,000; about 1,300,000; about 1,350,000; about 1,400,000; about 1,450,000; or about 1,500,000; about 1,200,000; about 1,250,000; about 1,300,000; about 1,350,000; about 1,400,000; about 1,450,000; or about 1,500,000 U.S.P. units/dose along with all values in between per dose and where the ratio of protease to lipase is such that the amount of lipase is never more than 0.188 times the amount of protease and where the ratio of protease activity to amylase activity is between 1:0.1 and 1:10.

In yet another embodiment, the digestive enzyme composition comprises at least one protease and at least one lipase, wherein the ratio of total proteases to total lipases (in U.S.P. units/dose) ranges from about 5.371:1 to about 20:1 including 5.371:1, 6:1, 7.1, 8.1, 9:1, 10:1, 11:1, 12:1, 13:1, 14:1, 15:1, 16:1, 17:1, 18:1, 19:1, and 20:1, along with all values in-between. In another embodiment, the digestive enzyme composition comprises at least one protease and at least one lipase, wherein the ratio of total proteases to total lipases (in U.S.P. units/dose) ranges from about 1:1 to about 20:1. In yet another embodiment, the ratio of proteases to lipases ranges from about 4:1 to about 10:1. In one embodiment, the ratio of proteases to lipases ranges from about 5.371:1 to about 10:1 including 5.371:1, 6:1, 7:1, 8:1, 9:1, and 10:1 along with all values in-between. In yet another embodiment, the ratio of proteases to lipases ranges from about 1:3 to about 1:0.57. In another embodiment, the ratio of proteases to lipases ranges from about 1:4 to about 1:0.05

In one embodiment, the digestive enzyme composition comprises at least one protease and at least one amylase, wherein the ratio of total proteases to total amylases (in U.S.P. units/dose) ranges from about 1:0.1 to about 1:10 including 1:0.25, 1:0.5, 1:0.75, 1:1, 1:1.25, 1:1.5, 1:1.75: 1:2, 1:1.25, 1:1.5, 1:1.75, 1:1.2, 1:1.25, 1:1.5, 1:1.75, 1:1.2, 1:1.25, 1:1.5, 1:1.75, 1:1.2: 1:1.5, 1:2, 1:3, 1:4, 1:5, 1:6, 1:7, 1:8, 1.9 and 1:10 along with all values in-between.

In another embodiment, the digestive enzyme composition comprises at least one protease and at least one amylase, wherein the ratio of total proteases to total amylases (in U.S.P. units/dose) ranges from about 1:6 to about 1:0.14. In another embodiment, the ratio of proteases to amylases ranges from about 1:7 to about 1:0.125.

In yet another embodiment, the digestive enzyme composition comprises at least one protease wherein the activity of protease is from about 5,000; about 7,500; about 10,000; about 15,000; about 20,000; about 25,000; about 30,000; about 35,000; about 40,000; about 50,000; about 55,000; about 65,000; about 70,000; about 75,000; about 90,000; about 95,000; about 100,000; about 110,000; about 115,000; about 130,000; about 140,000; about 140,400; about 150,000; about 155,000; about 160,000; about 170,000; about 175,000; about 180,000; about 190,000; about 195,000; about 200,000; about 210,000; about 220,000; about 230,000; about 240,000; about 250,000; about 280,000; about 290,000; about 300,000; about 310,000; about 320,000; about 330,000; about 340,000; about 350,000; about 360,000; about 370,000; about 380,000; about 390,000; about 400,000; about 410,000; about 420,000; about 430,000; about 440,000; about 450,000; about 465,000; about 470,000; about 480,000; about 490,000; about 500,000; about 510,000; about 520,000; about 530,000; about 540,000; about 550,000; about 560,000; about 570,000; about 580,000; about 590,000; about 600,000; about 610,000; about 620,000; about 630,000; about 640,000; about 650,000; about 660,000; about 670,000; about 680,000; about 690,000; about 700,000; about 710,000; about 720,000; about 730,000; about 740,000; about 750,000; about 760,000; about 770,000; about 780,000; about 790,000; about 800,000; about 810,000; about 820,000; about 830,000; about 840,000; about 850,000; about 860,000; about 870,000; about 880,000; about 890,000; about 900,000; about 910,000; about 920,000; about 930,000; about 940,000; about 950,000; about 960,000; about 970,000; about 980,000; about 990,000; about 1,000,000; about 1,010,000; about 1,020,000; about 1,020,000; about 1,030,000; about 1,040,000; about 1,050,000; about 1,060,000; about 1,070,000; about 1,080,000; about 1,090,000; about 1,100,000; about 1,100,000; about 1,120,000; about 1,130,000; about 1,140,000; about 1,150,000; about 1,170,000; about 1,190,000; about 1,200,000; about 1,210,000; about 1,250,000; about 1,300,000; about 1,350,000; about 1,400,000; about 1,450,000; or about 1,500,000; about 1,200,000; about 1,250,000; about 1,300,000; about 1,350,000; about 1,400,000; about 1,450,000; or about 1,500,000 U.S.P. units/dose along with all values in between per dose.

In yet another embodiment, the digestive enzyme composition comprises only one or more coated or uncoated proteases, wherein the activity of protease is between 10,000 to 1,500,000; from about 25,000 to about 1,000,000; from about 50,000 to about 750,000; from about 75,000 to about 500,000; from about 85,000 to about 250,000; from about 95,000 to about 200,000; or from about 110,000 to about 150,000 U.S.P. units/dose. Compositions may contain an amount of protease including, but not limited to, about 5,000; about 7,500; about 10,000; about 15,000; about 20,000; about 25,000; about 30,000; about 35,000; about 40,000; about 50,000; about 55,000; about 65,000; about 70,000; about 75,000; about 90,000; about 95,000; about 100,000; about 110,000; about 115,000; about 130,000; about 140,000; about 140,400; about 150,000; about 155,000; about 160,000; about 170,000; about 175,000; about 180,000; about 190,000; about 195,000; about 200,000; about 210,000; about 220,000; about 230,000; about 240,000; about 250,000; about 280,000; about 290,000; about 300,000; about 310,000; about 320,000; about 330,000; about 340,000; about 350,000; about 360,000; about 370,000; about 380,000; about 390,000; about 400,000; about 410,000; about 420,000; about 430,000; about 440,000; about 450,000; about 465,000; about 470,000; about 480,000; about 490,000; about 500,000; about 510,000; about 520,000; about 530,000; about 540,000; about 550,000; about 560,000; about 570,000; about 580,000; about 590,000; about 600,000; about 610,000; about 620,000; about 630,000; about 640,000; about 650,000; about 660,000; about 670,000; about 680,000; about 690,000; about 700,000; about 710,000; about 720,000; about 730,000; about 740,000; about 750,000; about 760,000; about 770,000; about 780,000; about 790,000; about 800,000; about 810,000; about 820,000; about 830,000; about 840,000; about 850,000; about 860,000; about 870,000; about 880,000; about 890,000; about 900,000; about 910,000; about 920,000; about 930,000; about 940,000; about 950,000; about 960,000; about 970,000; about 980,000; about 990,000; about 1,000,000; about 1,010,000; about 1,020,000; about 1,020,000; about 1,030,000; about 1,040,000; about 1,050,000; about 1,060,000; about 1,070,000; about 1,080,000; about 1,090,000; about 1,100,000; about 1,100,000; about 1,120,000; about 1,130,000; about 1,140,000; about 1,150,000; about 1,170,000; about 1,190,000; about 1,200,000; about 1,210,000; about 1,250,000; about 1,300,000; about 1,350,000; about 1,400,000; about 1,450,000; or about 1,500,000; about 1,200,000; about 1,250,000; about 1,300,000; about 1,350,000; about 1,400,000; about 1,450,000; or about 1,500,000 U.S.P. units/dose along with all values in between per dose. An added benefit is that this formulation will be useful in very young infants who are not able to tolerate lipase activity.

Provided herein is a pharmaceutical composition comprising digestive enzymes for use in the methods described herein, wherein the digestive enzymes comprise about 23,000 U.S.P. units/dose of lipase, about 144,000 U.S.P. units/dose of amylase and about 140,000 U.S.P. units/dose of protease. Provided herein is a pharmaceutical composition comprising digestive enzymes for use in the methods described herein, wherein the digestive enzymes comprise about 23,040 U.S.P. units/dose of lipase, about 144,000 U.S.P. units/dose of amylase and about 140,400 U.S.P. units/dose of protease. Provided herein is a pharmaceutical composition comprising digestive enzymes for use in the methods described herein, wherein the digestive enzymes comprise about 16,800 U.S.P. units/dose of lipase, about 70,000 U.S.P. units/dose of protease, and about 70,000 U.S.P. units/dose of amylase. Provided herein is a pharmaceutical composition comprising digestive enzymes for use in the methods described herein, wherein the digestive enzymes comprise about 16,800 U.S.P. units/dose of lipase, about 110,000 U.S.P. units/dose of protease, and about 70,000 U.S.P. units/dose of amylase.

Compositions contemplated herein include, but are not limited to, pellets, capsules, caplets, beadlets, sprinkles, tablets, softgels or other carriers.

Provided herein are dose forms of a composition provided herein. Non-limiting exemplary doses are set forth in Table 1.

| Dose | Supplemented with Protease |
|---|---|
| Lipase, U.S.P. units 8,400 | Protease, U.S.P. units 0; Protease, U.S.P. units 20,000; |
| Protease, U.S.P. units 35,000 | Protease, U.S.P. units 40,000; Protease, U.S.P. units |
| Amylase, U.S.P. units 35,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 16,800 | Protease, U.S.P. units 0; Protease, U.S.P. units 20,000; |
| Protease, U.S.P. units 70,000 | Protease, U.S.P. units 40,000; Protease, U.S.P. units |
| Amylase, U.S.P. units 70,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 33,600 | Protease, U.S.P. units 0; Protease, U.S.P. units 20,000; |
| Protease, U.S.P. units 140,000 | Protease, U.S.P. units 40,000; Protease, U.S.P. units |
| Amylase, U.S.P. units 140,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 50,400 | Protease, U.S.P. units 0; Protease, U.S.P. units 20,000; |
| Protease, U.S.P. units 210,000 | Protease, U.S.P. units 40,000; Protease, U.S.P. units |
| Amylase, U.S.P. units 210,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 67,200 | Protease, U.S.P. units 0; Protease, U.S.P. units 20,000; |
| Protease, U.S.P. units 280,000 | Protease, U.S.P. units 40,000; Protease, U.S.P. units |
| Amylase, U.S.P. units 280,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 84,000 | Protease, U.S.P. units 0; Protease, U.S.P. units 20,000; |
| Protease, U.S.P. units 350,000 | Protease, U.S.P. units 40,000; Protease, U.S.P. units |
| Amylase, U.S.P. units 350,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 100,800 | Protease, U.S.P. units 0; Protease, U.S.P. units 20,000; |
| Protease, U.S.P. units 420,000 | Protease, U.S.P. units 40,000; Protease, U.S.P. units |
| Amylase, U.S.P. units 420,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 117,600 | Protease, U.S.P. units 0; Protease, U.S.P. units 20,000; |
| Protease, U.S.P. units 490,000 | Protease, U.S.P. units 40,000; Protease, U.S.P. units |
| Amylase, U.S.P. units 490,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 134,400 | Protease, U.S.P. units 0; Protease, U.S.P. units 20,000; |
| Protease, U.S.P. units 560,000 | Protease, U.S.P. units 40,000; Protease, U.S.P. units |
| Amylase, U.S.P. units 560,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 151,200 | Protease, U.S.P. units 0; Protease, U.S.P. units 20,000; |
| Protease, U.S.P. units 630,000 | Protease, U.S.P. units 40,000; Protease, U.S.P. units |
| Amylase, U.S.P. units 630,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 168,000 | Protease, U.S.P. units 0; Protease, U.S.P. units 20,000; |
| Protease, U.S.P. units 700,000 | Protease, U.S.P. units 40,000; Protease, U.S.P. units |
| Amylase, U.S.P. units 700,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 184,800 | Protease, U.S.P. units 0; Protease, U.S.P. units 20,000; |
| Protease, U.S.P. units 770,000 | Protease, U.S.P. units 40,000; Protease, U.S.P. units |
| Amylase, U.S.P. units 770,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 201,600 | Protease, U.S.P. units 0; Protease, U.S.P. units 20,000; |
| Protease, U.S.P. units 840,000 | Protease, U.S.P. units 40,000; Protease, U.S.P. units |
| Amylase, U.S.P. units 840,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 218,400 | Protease, U.S.P. units 0; Protease, U.S.P. units 20,000; |
| Protease, U.S.P. units 910,000 | Protease, U.S.P. units 40,000; Protease, U.S.P. units |
| Amylase, U.S.P. units 910,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 235,200 | Protease, U.S.P. units 0; Protease, U.S.P. units 20,000; |
| Protease, U.S.P. units 980,000 | Protease, U.S.P. units 40,000; Protease, U.S.P. units |
| Amylase, U.S.P. units 980,000 | 60,000; Protease, U.S.P. units 80,000; Protease, U.S.P. |
| | units 100,000; Protease, U.S.P. units 120,000; Protease, |
| | U.S.P. units 140,000; or Protease, U.S.P. units 160,000. |
| Lipase, U.S.P. units 252,000 | Protease, U.S.P. units 0; Protease, U.S.P. units 20,000; |
| Protease, U.S.P. units | Protease, U.S.P. units 40,000; Protease, U.S.P. units |
| 1,050,000 | 60,000; Protease, U.S.P. units 80,000; Protease, U.S.P. |
| Amylase, U.S.P. units | units 100,000; Protease, U.S.P. units 120,000; Protease, |
| 1,050,000 | U.S.P. units 140,000; or Protease, U.S.P. units 160,000. |

Provided herein is a dose of a pharmaceutical composition that comprises digestive enzymes for use in the methods described herein, wherein the dose comprises about 8,400 U.S.P. units lipase, 35,000 U.S.P. units protease, and about 35,000 U.S.P. units amylase. Provided herein is a dose of a pharmaceutical composition comprising digestive enzymes for use in the methods described herein, wherein the dose comprises about 16,800 U.S.P. units lipase, about 70,000 U.S.P. units protease, and about 70,000 U.S.P. units amylase. Provided herein is a dose of a pharmaceutical composition comprising digestive enzymes for use in the methods described herein, wherein the dose comprises about 33,600 U.S.P. units lipase, about 140,000 U.S.P. units protease, and about 140,000 U.S.P. units amylase. Provided herein is a dose of a pharmaceutical composition comprising digestive enzymes for use in the methods described herein, wherein the dose comprises about 50,400 U.S.P. units lipase, about 210,000 U.S.P. units protease, and about 210,000 U.S.P. units amylase. Provided herein is a dose of a pharmaceutical composition comprising digestive enzymes for use in the methods described herein, wherein the dose comprises about 67,200 U.S.P. units lipase, about 280,000 U.S.P. units protease, and about 280,000 U.S.P. units amylase. Provided herein is a dose of a pharmaceutical composition comprising digestive enzymes for use in the methods described herein, wherein the dose comprises about 84,000 U.S.P. units lipase, about 350,000 U.S.P. units protease, and about 350,000 U.S.P. units amylase. Provided herein is a dose of a pharmaceutical composition comprising digestive enzymes for use in the methods described herein, wherein the dose comprises about 100,800 U.S.P. units lipase, about 420,000 U.S.P. units protease, and about 420,000 U.S.P. units amylase. Provided herein is a dose of a pharmaceutical composition comprising digestive enzymes for use in the methods described herein, wherein the dose comprises about 117,600 U.S.P. units lipase, about 490,000 U.S.P. units protease, and about 490,000 U.S.P. units amylase. Provided herein is a dose of a pharmaceutical composition comprising digestive enzymes for use in the methods described herein, wherein the dose comprises about 134,400 U.S.P. units lipase, about 560,000 U.S.P. units protease, and about 560,000 U.S.P. units amylase. Provided herein is a dose of a pharmaceutical composition comprising digestive enzymes for use in the methods described herein, wherein the dose comprises about 151,200 U.S.P. units lipase, about 630,000 U.S.P. units protease, and about 630,000 U.S.P. units amylase. Provided herein is a dose of a pharmaceutical composition comprising digestive enzymes for use in the methods described herein, wherein the dose comprises about 168,000 U.S.P. units lipase, about 700,000 U.S.P. units protease, and about 700,000 U.S.P. units amylase. Provided herein is a dose of a pharmaceutical composition comprising digestive enzymes for use in the methods described herein, wherein the dose comprises about 184,800 U.S.P. units lipase, about 770,000 U.S.P. units protease, and about 770,000 U.S.P. units amylase. Provided herein is a dose of a pharmaceutical composition comprising digestive enzymes for use in the methods described herein, wherein the dose comprises about 201,600 U.S.P. units lipase, about 840,000 U.S.P. units protease, and about 840,000 U.S.P. units amylase. Provided herein is a dose of a pharmaceutical composition comprising digestive enzymes for use in the methods described herein, wherein the dose comprises about 218,400 U.S.P. units lipase, about 910,000 U.S.P. units protease, and about 910,000 U.S.P. units amylase. Provided herein is a dose of a pharmaceutical composition comprising digestive enzymes for use in the methods described herein, wherein the dose comprises about 235,200 U.S.P. units lipase, about 980,000 U.S.P. units protease, and about 980,000 U.S.P. units amylase. Provided herein is a dose of a pharmaceutical composition comprising digestive enzymes for use in the methods described herein, wherein the dose comprises about 252,000 U.S.P. units lipase, about 1,050,000 U.S.P. units protease, and about 1,050,000 U.S.P. units amylase. Any of the doses may be optionally supplemented with additional protease. In one embodiment, a dose may be supplemented with about 0 U.S.P. units; about 20,000 U.S.P. units; about 40,000 U.S.P. units; about 60,000 U.S.P. units; about 80,000 U.S.P. units; about 100,000 U.S.P. units; about 120,000 U.S.P. units; about 140,000 U.S.P. units; or about 160,000 U.S.P. units protease.

A dose of a pharmaceutical composition described herein may be formulated for oral administered in an amount of, for example, about 3 teaspoons, about 2.75 teaspoons, about 2.5 teaspoons, about 2.25 teaspoons, about 2 teaspoons, about 1.75 teaspoons, about 1.5 teaspoons, about 1.25 teaspoons, about 1 teaspoon, about ½ teaspoon, about ¼ teaspoon, or about ⅛ teaspoon.

Alternatively, a dose of a pharmaceutical composition described herein may be formulated for oral administered in an amount of, for example, about ½ tablet, about 1 tablet, about 1.5 tablets, about 2 tablets, about 2.5 tablets, about 3 tablets, about 3.5 tablets, about 4 tablets, about 4.5 tablets, about 5 tablets, about 5.5 tablets, or about 6 tablets.

Provided herein is a single composition that comprises a dose of Formulation A and a dose of Formulation B. Compositions contemplated herein include, but are not limited to, pellets, capsules, caplets, beadlets, sprinkles, tablets, softgels, or other carriers.

In one embodiment, a composition comprises a dose of about ¼ teaspoon of formulation A comprises about 16,800 U.S.P. units lipase, about U.S.P. units 70,000 protease, and about 70,000 U.S.P. units amylase. In another embodiment, a composition comprises a dose of about ⅛ teaspoon of formulation A and about ½ teaspoon of formulation B, where the composition comprises about 8,400 U.S.P. units lipase, about 55,000 U.S.P. units protease, about U.S.P. units 35,000 amylase. In one embodiment, a composition comprises a dose of about ⅛ teaspoon of formulation A and about ⅛ teaspoon of formulation B, where the composition comprises about 8,400 U.S.P. units lipase, about 75,000 U.S.P. units protease, and about 35,000 U.S.P. units amylase. In one embodiment, a composition comprises a single dosage form of a composition dose of about ¼ teaspoon of formulation A and ½ teaspoon of formulation B, where the composition comprises about 16,800 U.S.P. units lipase, about U.S.P. units 90,000 protease, and about 70,000 U.S.P. units amylase.

In yet another embodiment, the digestive enzyme composition comprises only one more protease and only one or more amylases, which may be coated or uncoated, wherein the ratio of total proteases to total amylases (in U.S.P. units/dose) ranges from about 1:0.1 to about 1:10 including 1:0.25, 1:0.5, 1:0.75, 1:1, 1:1.25, 1:1.5, 1:1.75: 1:2, 1:1.25, 1:1.5, 1:1.75, 1:1.2, 1:1.25, 1:1.5, 1:1.75, 1:1.2, 1:1.25, 1:1.5, 1:1.75, 1:1.2: 1:1.5, 1:2, 1:3, 1:4, 1:5, 1:6, 1:7, 1:8, 1.9 and 1:10 along with all values in-between. In another embodiment, the digestive enzyme composition comprises at least one protease and at least one amylase, wherein the ratio of total proteases to total amylases (in U.S.P. units/dose) ranges from about 1:6 to about 1:0.14. In another embodiment, the ratio of proteases to amylases ranges from about 1:7 to about 1:0.125. An added benefit is that this formulation will be useful in very young infants who are not able to tolerate lipase activity.

In one embodiment the coated or uncoated digestive enzymes to be administered are comprised of pancreatin, pancrealipase, or a combination thereof. In one embodiment a coating technology can be used, such as the ones described in U.S. Pat. Nos. 6,835,397, 6,153,236, or US 2009-0004285 which are herein incorporated by reference in their entirety.

Enzyme preparations with non-lipid enteric coatings can be used to deliver lipases in individuals in need of lipase administration. Certain methods and enzyme compositions for use in treating children and other individuals in, for example, U.S. Pat. Nos. 7,138,123, 6,660,831, 6,632,429, 6,534,063, which is herein incorporated by reference in its entirety.

The composition of the dosage form may include other components, generally utilized in pharmaceutical preparations including but not limited to binders, disintegrants, extracts, lubricants, fillers, flavorings, preservatives, colorants, taste maskers, diluents and coating agents, such as vegetable oil, crystalline oils, and other coating methodologies.

In one embodiment, coating of a digestive enzyme preparation is used to obtain release at selected transit times or in selected locations of the gastrointestinal tract of humans. In one aspect, this disclosure relates to controlled release enzyme preparations administered to a subject with Celiac disease or a related disorder.

In yet another aspect, this disclosure relates to an enzyme delivery system comprising a coated enzyme preparation having particles which comprise: (a) a core comprising pancreatic or digestive enzymes present in an amount from about 5% to 99% by weight of the particles; and (b) a generally uniform coating to provide for controlled release of the enzymes, the coating comprising an emulsifiable lipid. In one aspect, the coated enzyme preparation particles of the enzyme delivery system are non-aerosolizable.

In some embodiments a coated digestive enzyme preparation comprising (a) a core containing a digestive enzyme particle, where the enzyme present in an amount of from about 5% to 95% by weight of the particles, including 5%, 10%, 15%, 20%, 25%, 30%, 35%, 40%, 45%, 50%, 55%, 60%, 65%, 70%, 75%, 80%, 85%, 90% and 95% by weight, along with all values in-between; and (b) a coating comprising a crystallizable lipid, wherein the coating continuously coats the core and the crystallizable lipid releases the enzyme upon exposure to physiological conditions.

In some embodiments a coated enzyme preparation having particles which comprise: (a) a core comprising pancreatic or other digestive enzymes present in an amount of from about 5% to 95% by weight of the particles, including 5%, 10%, 15%, 20%, 25%, 30%, 35%, 40%, 45%, 50%, 55%, 60%, 65%, 70%, 75%, 80%, 85%, 90% and 95% by weight along with all values in-between; and (b) a generally uniform coating to provide for controlled release of the enzymes, the coating comprising a crystallizable lipid. In some embodiments, the coated enzyme preparation particles of the enzyme delivery system are non-aerosolizable.

The present disclosure also relates to methods of making the enzyme preparations by lipid coating and/or encapsulation of digestive enzymes. The methods comprise providing an emulsifiable lipid and coating pancreatic/digestive enzyme particles with the lipid. The digestive enzymes comprise 5 to 99% of the coated enzyme preparations by weight.

In another aspect as described herein, the inventors have discovered that the methods of this disclosure produce coated digestive enzyme preparations comprising digestive and/or pancreatic enzymes coated with an emulsifiable lipid alone, or with a lipid blend to achieve a controlled rate of enzyme release, with increased release of the pancreatic/digestive enzyme upon exposure of the coated preparation to a suitable solvent. The inventors have discovered that coated pancreatic/digestive enzyme preparations having a coating consisting essentially of one or more monoglycerides exhibit time-sensitive biologically-suitable release of the pancreatic/digestive enzymes upon exposure of the coated composite to a solvent, such as water, while protecting against release in 0.1 N HCl or acidic gastric fluid.

The nature of the human digestive tract creates challenges for the delivery of digestive enzymes to patients susceptible to treatment with digestive enzymes. Multiple temperature and pH changes over the course of the digestive tract make specific delivery a challenge but a necessity. For instance, pH as low as 1 is encountered in the stomach, but rapidly increases to a more basic pH of 5-6 in the proximal small intestine. For example, generally the pH in the stomach is approximately 1.2, the pH in the duodenum is about 5.0 to 6.0; the pH in the jejunum is about 6.8, and the pH is about 7.2 in the proximal ileum and about 7.5 in the distal ileum. The low pH in the stomach that changes rapidly to a more basic pH of 5-6 in the proximal small intestines calls for a specific delivery method depending upon where the enzyme is to be delivered.

Delivery of digestive enzymes can also be challenging due to the rapid degradation and denaturing of enzymes at ambient room temperature, as well as the enhanced degradation and denaturing that can occur with high temperature, pressure, humidity and/or exposure to light. Moisture and heat together can quickly destabilize enzymes, reducing their effectiveness, and weaken their potency leading to inaccurate dosing and shortened shelf life. Denaturation or destabilization of the enzymes can reduce their effectiveness by reducing the dose of active enzymes to less than the amount needed for effective treatment. In one embodiment, to protect and stabilize the pancreatic/digestive enzyme from unfavorable conditions such as oxidation, the pancreatic/digestive enzyme (core) is coated or encapsulated in a continuous coating containing an emulsifiable lipid. In another aspect, this disclosure provides new coated enzyme preparations with improved shelf life.

Manufacturers of enzyme preparations have used enteric coatings to deliver lipases in individuals requiring administration of lipases. Because the porcine enzymes are delivered in a mixture of proteases, lipases and amylases, and because these compositions for human consumption were prepared for lipase delivery, the uses of these enteric coatings, which include such substances as hypromellose phthalate, dimethicone 1000, and dibutyl phthalate, preclude delivery of proteases at the proper location for protein digestion, which is the duodenum. All other enzyme preparations presently on the market contain at least one of these enteric coating substances and/or other additives in the preparation.

In one embodiment the present disclosure includes a coated digestive enzyme preparation and/or composite, which in some embodiments is an encapsulated pancreatic/digestive enzyme preparation. In other aspects, the disclosure includes enzyme delivery systems and pharmaceutical compositions comprising coated pancreatic/digestive enzyme preparations. These coated or encapsulated enzyme preparations contain cores comprising pancreatic or digestive enzyme particles, and a coating comprising an emulsifiable lipid.

The coatings in the digestive/pancreatic enzyme preparations create a barrier to degradation and denaturation and to allow more accurate levels of active enzymes to be utilized by treated individuals. The lipid coating of this disclosure provides a significant barrier to moisture, humidity and exposure to light by allowing for a physical barrier as well as one that prevents and/or reduces hydrolysis. The coated enzyme preparations undergo less hydrolysis as a result of protection from moisture in the environment by the lipid coating. As a result of the present disclosure, pancreatic/digestive enzymes are provided which can tolerate storage conditions (e.g., moisture, heat, oxygen, etc.) for long periods of time thus enabling extended shelf life. The coating of the encapsulated enzyme preparation protects the enzyme from the environment and provides emulsification in a solvent without detracting from the abrasion resistance of the coating. The disclosure thus further relates to more stable enzyme preparations.

It is another aspect of the present disclosure to make an enzyme preparation without the use of extenders colorants, dyes, flow enhancers and other additives to reduce the potential for allergens and other sensitivity reactions in children and other treated individuals. It has been discovered that in some embodiments, the digestive enzymes can be encapsulated with a single lipid excipient to improve retention of enzyme activity, ease of administration, tolerability, and safety of administration, among other properties. Surprisingly, digestive enzyme particles containing lipases can be successfully encapsulated with coating consisting essentially of only hydrogenated soy oil.

Porcine pancreatic/digestive enzymes possess a significant odor and taste, similar to cured or smoked pork. This taste and smell can be strong and offensive to some individuals taking enzyme replacement, and especially to children. In one embodiment, the addition of a lipid coating provides significant odor and taste masking to the enzyme preparation, which allows for the tolerance of taste, as the lipid coating is odorless and tasteless. The use of this method of taste masking not involving the use of color, dyes, perfumes or other substances is preferable for the administration of medications, which have an unpleasant or undesirable taste and odor. In another embodiment, this disclosure relates to coated digestive enzyme preparations with improved taste and odor.

In some embodiments, the coatings on the digestive enzyme particle cores are preferably continuous coatings. By “continuous”, it is meant that the pancreatic/digestive enzyme is completely surrounded. The continuous coating fully surrounds or encapsulates the pancreatic/digestive enzymes. The encapsulation provides protection of the pancreatic/digestive enzyme from conditions such as moisture and oxidation.

In the manufacture of pharmaceuticals, encapsulation refers to a range of techniques used to enclose medicines in a relatively stable shell known as a capsule, allowing them to, for example, be taken orally or be used as suppositories. “Encapsulate” as used herein means that the coating completely surrounds the pancreatic/digestive enzyme. A coated or encapsulated preparation may contain one or more digestive enzyme particles enveloped in one coating to form one coated or encapsulated digestive enzyme particle in the coated or encapsulated digestive enzyme preparation.

The two main types of capsules are hard-shelled capsules, which are normally used for dry, powdered ingredients, and soft-shelled capsules, primarily used for oils and for active ingredients that are dissolved or suspended in oil. Both of these classes of capsule are made both from gelatin and from plant-based gelling substances like carrageenans and modified forms of starch and cellulose, and the latter form is usually seamless. Capsules are made in two parts by dipping metal rods in molten gelatin solution. The capsules are supplied as closed units to the pharmaceutical manufacturer. Before use, the two halves are separated, the capsule is filled with powder (either by placing a compressed slug of powder into one half of the capsule, or by filling one half of the capsule with loose powder) and the other half of the capsule is pressed on. The advantage of inserting a slug of compressed powder is that control of weight variation is better, but the machinery involved is more complex.

Sprinkle capsules are a dosage form consisting of small beads or granules of an active drug contained in a capsule that can be readily administered by simply opening up the capsule and distributing the contents over something to be swallowed.

In addition, the encapsulation also provides controlled release of the pancreatic/digestive enzyme. In one embodiment, the emulsification properties of the coating in a solvent allows for controlled release of the enzyme in the gastrointestinal (GI) system, preferably the region of the GI tract where the enzymes are to be utilized. For example, for conditions requiring treatment with proteases, the release of the protease portion of the enzymes is necessary in the proximal small intestine, thereby necessitating a lipid encapsulation, which has a dissolution profile showing a release of between 10% to 100% of the active substance into solution over a time period of between 30 and 90 minutes. In one embodiment, the dissolution profile shows a release of about 20%, 30%, 40%, 50%, 60%, 70%, 80%, 90% or 100%, and all values in between, of the coated substance into solution over a time period of about 30, 35, 40, 45, 50, 55, 60, 65, 70, 75, 80, 85 or 90 minutes and all values in between. Dissolution profiles may be obtained using methods and conditions known to those of skill in the art. For example, dissolution profiles can be determined at various pHs, including pH 1, 2, 3, 4, 5, 6, 7, 8, 9, 10 and all values in between.

The rate of release of the bioactive substance can also be controlled by the addition of additives as described below. When the preparations are exposed to a solvent, the solvent interacts with the mollifiable lipid in the coating and results in emulsification of the coating and release of the bioactive substance.

A suspension is a heterogeneous fluid containing solid particles that are sufficiently large for sedimentation. Usually, they must be larger than 1 micrometer. The internal phase (solid) is dispersed throughout the external phase (fluid) through mechanical agitation, with the use of certain excipients or suspending agents. Unlike colloids, suspensions will eventually settle. An example of a suspension would be sand in water. The suspended particles are visible under a microscope and will settle over time if left undisturbed. This distinguishes a suspension from a colloid in which the suspended particles are smaller and do not settle. Colloids and suspensions are different from a solution, in which the dissolved substance (solute) does not exist as a solid and solvent and solute are homogeneously mixed. Oftentimes, powders of active ingredients may be packaged such that the addition of a diluent dissolves the powder and holds it in a liquid suspension.

A pharmaceutical preparation may be prepared in which an excipient provides a matrix to capture and protect a product before delivery. Pharmaceutical preparations may be prepared whereby the individual who takes the preparation has a reduction in the number of capsules/tablets per dosage; i.e., the preparation is stabilized and may contain a therapeutically effective amount of a protease, an amylase, and/or a lipase. Preparations may include, for example, a stabilizing matrix consisting essentially of a solidified microcrystalline cellulose which captures and protects therapeutically effective amounts of digestive enzyme particles within the stabilizing matrix. This can be done, for example, through the use of what is known in the art as PROSOLV® technology.

PROSOLV® is a combination of excipients which allow for optimized flow, compaction and product uniformity. This technology allows for uniformity in this combination, as well as manufacturing a very small tablet which would be amenable for children. With PROSOLV® technology, the ingredients are not just blended, but are co-processed, which assures that equal particles are uniformly distributed and these results are easily reproducible. This allows for stability and superb product quality.

Whether utilizing the PROSOLV® method or other methodology, the one or more digestive enzymes will be formulated and manufactured such that the particles will be uniformly distributed and there will be no overage with respect to the amount of enzyme found in the preparation. Said new drug formulation can be found in, but is not limited to, formulations which include digestive/pancreatic enzymes with and without the utilization of the PROSOLV® technology.

In a further embodiment, a direct compression method may be used for the manufacture of a pharmaceutical tablet preparation including the steps of: (a) forming an active blend by blending an intimate admixture of silicified microcrystalline cellulose and a therapeutic agent comprising one or more digestive enzymes; (b) forming a color blend by blending an intimate admixture of one or more pharmaceutically acceptable dyes and silicified microcrystalline cellulose if color is necessary; (c) combining the active blend, the color blend and a disintegrant into a pre-blend; (d) adding a lubricant to the pre-blend to form a final blend; and (e) compressing the final blend to form a pharmaceutical tablet preparation or a mixture of time released microtabs or a time released tablet.

This may be accomplished by combining the digestive enzymes with one of the patented PROSOLV® technologies, i.e.: PROSOLV® SMCC 50 or PROSOLV® SMCC 90, or other PROSOLV® technologies. When employing the PROSOLV® method, the silicified microcrystalline cellulose (SMCC) used in the preparation of the present invention may be any commercially available combination of microcrystalline cellulose granulated with colloidal silicon dioxide. The SMCC generally will be as described in Sherwood et al, Pharm. Tech., October 1998, 78-88 and U.S. Pat. No. 5,585,115, which is incorporated herein by reference in its entirety. SMCC can be obtained commercially from Edward Mendell Company, Inc., a subsidiary of Penwest Ltd., under the name PROSOLV® SMCC. There are different grades of SMCC available, with particle size being the differentiating property among the grades. For example, PROSOLV® SMCC 90 has a median particle size, by sieve analysis, in the region of 90 micrometers. PROSOLV® SMCC 50 has a median particle size, by sieve analysis, in the region of about 40-50 micrometers.

A pharmaceutical composition described herein may be prepared using a direct compression method, a dry granulation method, or by wet granulation. Preferably, the digestive/pancreatic enzyme preparation may be prepared using a direct compression process. This preferred process consists of two main steps: blending and compression.

The blending step is composed of an active blend, color blend, pre-blend, and final blend (lubrication). The formulation of the present invention may include a number of other ingredients for optimal characteristics of the pharmaceutical composition. Such other ingredients and the amounts to be used are within the knowledge of one in the art and are known in the pharmaceutical arts. These may include disintegrates, lubricants and/or coloring agents among others. Suitable disintegrants include, for example, sodium starch glycolate, other starches such as pregelatinized starch, and celluloses. Suitable lubricants may be provided, such as magnesium stearate, calcium stearate, talc and stearic acid. Any coloring agent certified by the FDA may be used, such as FD&C Yellow #6, among others.

When used as a pharmaceutical preparation, elixirs contain an active ingredient that is dissolved in a solution that contains some percentage (usually 40-60%) of ethyl alcohol and is designed to be taken orally.

Syrups are oftentimes employed as a base for medicinal purposes and consist of a concentrated or saturated solution of refined sugar in distilled water.

A suspension of liquid droplets or fine solid particles in a gas is called an aerosol. This can take the form of an oral spray.

A gum may be devised whereby an active ingredient is incorporated into a vegetative resinous substance (e.g., acacia) and released via the actual mechanical effect of chewing or the action of saliva on the gum itself.

A thin strip is an active pharmaceutical product coated by a lipid layer designed to dissolve in the mouth over a brief period of time. The same technology could be used to produce a medicated lollipop for transmucosal delivery.

In pharmaceutical terms, a granule is a small particle gathered into a larger, permanent aggregate in which the original particles can still be identified.

In some aspects, the disclosure relates to the production of selected coated enzyme preparations made by coating digestive enzyme particles with lipids not previously used in coated digestive enzyme preparations. The unique mixtures of emulsifiable lipids and enzymes can deliver certain components of the pancreatic/digestive enzymes to selected locations and/or at selected times during transit of the GI tract. In some aspects, the disclosure relates to methods of delivering digestive enzymes to humans based upon dissolution profiles.

The emulsifiable lipid may be any lipid, lipid mixture, or blend of lipid and emulsifiers which emulsifies when exposed to a solvent, and has a melting point which allows the lipid to be a solid at typical storage temperatures. The emulsifiable lipid can be a vegetable or animal derived-lipid. In another embodiment, the emulsifiable lipid consists essentially of, or comprises one or more monoglycerides, diglycerides or triglycerides, or other components including, for example, emulsifiers found in hydrogenated vegetable oils. In another embodiment the lipid is a non-polar lipid.

As used herein, animal and/or vegetable “derived” lipids can include fats and oils originating from plant or animal sources and/or tissues, and/or synthetically produced based on the structures of fats and oils originating from plant or animal sources. Lipid material may be refined, extracted or purified by known chemical or mechanical processes. The lipid may, in one embodiment, comprise a Type I U.S.P.-National Formulary vegetable oil.

The digestive enzyme used in the present disclosure can be any combination of digestive enzymes of a type produced by the pancreas, including, but not limited to digestive enzymes from a pancreatic source or other sources. The scope of the disclosure is not limited to pancreatic enzymes of porcine origin, but can be of other animal or plant origin as well as those that are synthetically derived. In one embodiment, the digestive enzyme is derived from mammalian sources such as porcine-derived digestive enzymes. In another embodiment, the enzyme includes one or more enzymes, and is plant derived, synthetically derived, recombinantly produced in microbial, yeast, or mammalian cells, or includes a mixture of enzymes from one or more sources. For example, digestive enzymes may include one or more enzymes from one or more sources mixed together. This includes, for example, the addition of single digestive enzymes to digestive enzymes derived from pancreatic sources in order to provide appropriate levels of specific enzymes that provide more effective treatment for a selected disease or condition. One source of digestive enzymes can be obtained, for example, from Scientific Protein Laboratories. In one embodiment, the digestive enzyme is, for example a pancreatin/pancrelipase composition. In another embodiment, the digestive enzymes comprise or consist essentially of 25 U.S.P. units protease, 2 U.S.P. units lipase, and 25 U.S.P. units amylase per milligram. The term digestive enzyme may refer to one or more enzymes of a type produced by the pancreas.

In one embodiment, the digestive enzyme used present as consisting of particles having various sizes. In another embodiment, the particles of digestive enzyme are screened to obtain particles of a suitable size for encapsulation by removing particles that are too fine or too large. For example, the particles may be sieved to obtain particles of a suitable size or more uniform size range for encapsulation.

In one embodiment, the minimum amount of pancreatic enzyme present in the core is at least about 5% active enzymes by weight of the coated enzyme preparation, but in another embodiment is at least about 30%, or at least about 50% by weight. In one embodiment, the maximum amount of pancreatic/digestive enzyme present in the composite is at most about 99% by weight, and in another embodiment is at most about 98%, 95%, 90%, 85%, 80%, 75% or 70% of the coated enzyme preparation. In another embodiment, the amount of pancreatic enzyme present in the composite is about 10%, 15%, 20%, 25%, 35%, 40%, 45%, 55%, 60%, 65%, 70%, 72.5%, 75%, 77.5%, 80%, 82.5%, 87.5%, or 92.5% by weight or anywhere in between. At least about or at most about a % of enzyme may include equal to or about that % of enzyme. The term “about” includes equal to, and a range that takes into account experimental error in a given measurement. As used in connection with particle sizes, the term “about” can refer to plus or minus 10, 9, 8, 7, 6, 5, 4, 3, 2 or 1% or anywhere in between. As used in connection with % particles that can be sieved, the term “about” can refer to plus or minus 10, 9, 8, 7, 6, 5, 4, 3, 2 or 1% or anywhere in between.

In one embodiment, the composition which contains the encapsulated digestive enzyme preparation or composite is delivered as a sprinkle, powder, capsule, tablet, pellet, caplet or other oral form. In another embodiment, packaging the encapsulated enzyme preparations in an enzyme delivery system that further comprises single dose sachet-housed sprinkle preparations allows for ease of delivery and accurate dosing of the enzyme by allowing a specific amount of enzyme to be delivered in each dosing. Allowing for specific unit dosing of an enzyme preparation which maintains the enzyme activity within specific stability parameters is an enhancement over other sprinkle formulations, which are housed in a multi-unit dosing form that allows for air, moisture and heat to depredate and denature the enzyme preparation. In one embodiment, the powder or sachet is housed in a trilaminar pouch of which one layer is foil, or similar barrier to keep out moisture and to protect the enzyme preparation from adverse environmental factors. The disclosure further relates to an improvement in stability due to a reduction in hydrolysis due to the lipid encapsulation and composition of package.

In another embodiment, the lipid encapsulation methodology reduces the aerosolization of the enzyme preparation that may be caustic to the patient if inhaled. In another embodiment, the disclosure includes delivery of digestive enzymes with improved safety of administration, by reducing the amount of aerosolization of the enzyme. The lipid encapsulation reduces aerosolization and the potential for caustic burn, aspiration, and/or aspiration pneumonias in patients and administrators of the enzyme preparation, thereby reducing the potential for illness in already compromised children such as those with cystic fibrosis, and leading to safer administration.

As used herein, the term “non-aerosolizable” will be used to refer to a coated or encapsulated enzyme preparation where substantially all of the particles are large enough to eliminate or reduce aerosolization upon pouring of the coated enzyme preparation compared to uncoated enzyme particles.

As described and referred to herein, suitable pancreatic/digestive enzymes and suitable coatings may be used in the compositions and methods of this disclosure. The choice of suitable enzymes and of suitable lipid coatings, including choice of the type or amount of enzymes or coating, are guided by the specific enzyme needs of the individuals, and the selected diseases to be treated. The encapsulated enzyme preparations that are one aspect of this disclosure have not been previously described.

In another embodiment, the disclosure relates to a method of controlling the rate of release of the pancreatic/digestive enzyme from an encapsulated enzyme preparation upon exposure to a solvent. In one aspect, the method comprises blending an emulsifiable lipid with an amount of one or more additives to obtain a lipid blend and coating the digestive enzyme particle with the blend to form an encapsulated digestive enzyme preparation containing particles comprising a core which contains the enzyme, and a coating which contains the lipid. In one embodiment, the emulsifiable lipid is a blend where the emulsifiable lipid and additive are not the same, and where the rate of release of the enzyme from the encapsulated composite upon exposure to a solvent is decreased as the amount of additive is increased. In the alternative, the rate of release of the enzyme from the encapsulated composite upon exposure to a solvent is increased as the amount of additive is decreased.

The lipid coating surprisingly does not appear to be reduced or destroyed by hydrochloric acid (HCl) present in the stomach, thereby protecting the enzyme from degradation following administration until the enzyme preparation reaches its target region in the GI tract. Further the lipid coat reduces the exposure of the enzyme to attack by water, thereby reducing hydrolysis, and further protecting the digestive enzymes from degradation. In addition, the inventors have found that an excipient containing only lipid can be used to coat or encapsulate digestive enzyme particles containing lipase.

Enzyme preparations supplied by the API supplier may be provided as irregular shaped, and multi-sized particles, with uneven edges, and much clumping, and containing some crystalline salt particles. Uneven particle size and shape reduces flow properties, and interferes with packaging. In addition, pouring uncoated enzyme into the mouth of a subject would be difficult, and potentially may cause too much or too little of the enzyme to be delivered. In one embodiment, processing the digestive enzyme particles according to methods in accordance with one aspect of this disclosure yields a non-dusty, free-flowing particulate preparation suitable for sachet packaging and for pouring onto food or drink. In addition, as discussed throughout, the use of lipid encapsulation to prevent aerosolization and, therefore, increase safety, and to increase flow properties which enhance manufacturing of a pharmaceutical is an embodiment of the instant disclosure.

“Emulsifiable lipids” as used herein means those lipids that contain at least one hydrophilic group and at least one hydrophobic group, and have a structure capable of forming a hydrophilic and hydrophobic interface. These chemical and/or physical properties, mentioned above of an emulsifiable lipid permit emulsification. Examples of interfaces include, for example, micelles and bilayers. The hydrophilic group can be a polar group and can be charged or uncharged.

In one embodiment, the emulsifiable lipid is derived from animal or vegetable origins, such as, for example, palm kernel oil, soybean oil, cottonseed oil, canola oil, and poultry fat, including hydrogenated type I vegetable oils. In one embodiment, the lipid is hydrogenated. In another embodiment, the lipid is saturated or partially saturated. Examples of emulsifiable lipids include, but are not limited to, monoglycerides, diglycerides, fatty acids, esters of fatty acids, phospholipids, salts thereof, and combinations thereof.

The emulsifiable lipid is preferably a food grade emulsifiable lipid. Some examples of food grade emulsifiable lipids include sorbitan monostearates, sorbitan tristearates and calcium stearoyl lactylates. Examples of food grade fatty acid esters which are emulsifiable lipids include acetic acid esters of mono- and diglycerides, citric acid esters of mono- and diglycerides, lactic acid esters of mono- and diglycerides, polyglycerol esters of fatty acids, propylene glycol esters of fatty acids, and diacetyl tartaric acid esters of mono- and diglycerides. Lipids can include, for example, hydrogenated soy oil. Any emulsifiable lipid may be used in the methods and products of this disclosure. In one embodiment, the emulsifiable lipid used will produce non-agglomerating, non-aerosolizing enzyme preparation particles.

In another embodiment, the method relates to preparation of an encapsulated, controlled release digestive enzyme preparation with enhanced flow properties useful in the treatment of individuals with Celiac disease or a related disorder, the method comprising: a) blending an emulsifiable lipid with one or more additives to obtain a blend; and b) coating screened digestive enzyme with the blend to form an encapsulated digestive enzyme containing a core which contains the digestive enzyme and a coating which contains the blend of emulsifiable lipid.

The coating of the enzyme with the lipid allows for the enzyme to become more uniform in size and shape, but reduces the jagged edges associated with the raw enzyme, and allows for ease of administration and ease of packaging, as the flow properties associated with the covered enzyme will allow for the packaging machinery to easily fill the sachet/pouch with the enzyme and reduces overfilling or underfilling of the sachet.

In another embodiment, the disclosure relates to a method of controlling the rate of release of a digestive enzyme from the encapsulated preparation by using a lipid blend to coat the digestive enzyme. The method includes blending an emulsifiable lipid with one or more additives to obtain a blend, and coating the digestive enzyme with the blend to form an encapsulated digestive enzyme containing a core which contains the digestive enzyme and a coating which contains the blend of emulsifiable lipid. The rate of release of the enzyme from the encapsulated preparation upon exposure with a solvent is decreased as the amount of additive is increased. In the alternative, the rate of release of the enzyme from the encapsulated composite upon exposure with a solvent is increased as the amount of additive is decreased. Thus, the nature of the coating allows for controlled release of the enzyme from the encapsulate.

Different dosage forms have different benefits. Tablets and capsules are the most common dosage forms for oral administration due to ease of manufacture, packaging and administration. Different forms of tablets have been primarily devised to meet the needs of select populations while maintaining the integrity of the active pharmaceutical ingredient. Some populations, notably infants and young children, cannot swallow tablets or capsules or find it difficult to do so. In these instances, a tablet that dissolves under the tongue, in the mouth, or in a specified liquid, or one that could be harmlessly chewed would be beneficial. Capsules that could be opened and their contents sprinkled over a small amount of food or in a liquid would also be beneficial. Any improvement that eases the administration of a necessary medication or lessens the antagonism associated with said administration, without compromising the effectiveness of the active pharmaceutical ingredient, is worthwhile.

Other types of solid dosage forms such as thin strips, lollipops or gum bring a novel concept to the administration of medications to children. Aside from the obvious ease of administration from the viewpoint of the caregiver, there may be an added benefit. The administration of medication is oftentimes a private issue and the ability of a caregiver to provide a dose of medication in a seemingly matter-of-fact form may preserve that perception and instill in the user a mindset that views the administration as pleasant rather than monotonous or negative.

Liquid dosage forms also provide benefits of administration to infants and young children or anyone with compromised swallowing capability. Syrups, solutions and suspensions are easily swallowed. Unpleasant tastes can be masked by flavoring. An oral spray allows for the quick administration of a pre-measured dose of medication and supplies multiple metered doses in one handy device. With no need to aid swallowing (such as a glass of water, etc.) and the convenience of not having to rifle through a bottle of tablets, administration is simplified.

A tablet is a mixture of active substances and excipients, usually in powder form, pressed or compacted into a solid. The excipients include binders, glidants (flow aids) and lubricants to ensure efficient tableting; disintegrants to ensure that the tablet breaks up in the digestive tract; sweeteners or flavors to mask the taste of bad-tasting active ingredients; and pigments to make uncoated tablets visually attractive. A coating (sugar, enteric or film) may be applied to hide the taste of the tablet's components, to make the tablet smoother and easier to swallow, and to make it more resistant to the environment, extending its shelf life. Tablets may be buffered (by potassium metaphosphate, potassium phosphate, monobasic sodium acetate, etc.) to combat change in pH. Tablets may be delayed-release, sustained-release, extended-release, controlled-delivery, long-acting, orally-disintegrating or melts, among others, often denoting the pharmacokinetic profile of the active agent. A capsule-shaped tablet is a caplet.

Some tablets may be taken sublingually or allowed to dissolve in the mouth. The principle behind sublingual administration is simple. When a chemical comes in contact with the mucous membrane beneath the tongue, or buccal mucosa, it diffuses through it. Because the connective tissue beneath the epithelium contains a profusion of capillaries, the substance then diffuses into them and enters the venous circulation. Troches are medicated lozenges designed to dissolve in the mouth. Soluble tablets dissolve on contact with the tongue.

A slurry may be made when a dissolvable tablet containing a gelling agent is added to a liquid.

Tablets may also be micro-coated and placed in a capsule for administration.

The compositions described herein can be administered either alone or more typically in combination with one or more of a conventional pharmaceutical carrier, excipient buffer, stabilizer or the like. Such materials should be non-toxic and should not interfere with the efficacy of the active ingredient. The precise nature of the carrier or other material will depend on the route of administration. The term “excipient” is used herein to describe any ingredient other than the compound(s) (enzymes) used in the composition as described herein and known in the art.

Acceptable carriers are physiologically acceptable to the administered patient and retain the therapeutic properties of the compounds with/in which it is administered. Acceptable carriers and their formulations are and generally described in, for example, Remington' pharmaceutical Sciences (18th Edition, ed. A. Gennaro, Mack Publishing Co., Easton, Pa. 1990). Two exemplary carriers are water and physiological saline. The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject compounds from the administration site to a portion of the body. Each carrier is acceptable in the sense of being compatible with the other ingredients of the formulation and not injurious to a subject to whom it is administered. Nor should an acceptable carrier alter the specific activity of the subject compounds.

Acceptable carriers, excipients, or stabilizers are those that are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; and/or non-ionic surfactants such as TWEEN®, PLURONICS® or polyethylene glycol (PEG).

In one embodiment, coating of a digestive enzyme preparation is used to obtain release at selected transit times or in selected locations of the gastrointestinal tract of humans. In one aspect, this invention relates to controlled release enzyme preparations administered to a subject with Celiac disease or related disorders. In another aspect of the present invention one or more coatings are utilized to target delivery to the small intestines.

In yet another aspect, this invention relates to an enzyme delivery system comprising a coated enzyme preparation having particles which comprise: (a) a core comprising pancreatic or digestive enzymes present in an amount from about 5% to 95% by weight of the particles; and (b) a generally uniform coating to provide for controlled release of the enzymes, said coating comprising an emulsifiable lipid. In one aspect, the coated enzyme preparation particles of the enzyme delivery system are non-aerosolizable.

The present invention also relates to methods of making the enzyme preparations by lipid coating and/or encapsulation of digestive enzymes. The methods comprise providing an emulsifiable lipid, and coating optionally screened pancreatic/digestive enzyme particles with the lipid. The digestive enzymes comprise 5 to 95% of the coated enzyme preparations by weight.

In another aspect as described herein, the inventors have discovered that the methods of this invention produce coated digestive enzyme preparations comprising digestive and/or pancreatic enzymes coated with an emulsifiable lipid alone, or with a lipid blend to achieve a controlled rate of enzyme release, with increased release of the pancreatic/digestive enzyme upon exposure of the coated preparation to a suitable solvent. The inventors have discovered that coated pancreatic/digestive enzyme preparations having a coating consisting essentially of one or more monoglycerides exhibit increased release of the pancreatic/digestive enzymes upon exposure of the coated composite to a solvent, such as water, while protecting against release in 0.1 N HCl.

The nature of the human digestive tract creates challenges for the delivery of digestive enzymes to patients susceptible to treatment with digestive enzymes. Multiple temperature and pH changes over the course of the digestive tract make specific delivery a necessity and a challenge. For instance, pH as low as 1 is encountered in the stomach, but rapidly increases to a more basic pH of 5-6 in the proximal small intestine. For example, the pH in the stomach is approximately 1.2, the pH in the duodenum is about 5.0 to 6.0; the pH in the jejunum is about 6.8, and the pH is about 7.2 in the proximal ileum and about 7.5 in the distal ileum. The low pH in the stomach which changes rapidly to a more basic pH of 5-6 in the proximal small intestine calls for a specific delivery method depending upon where the enzyme is to be delivered.

Delivery of digestive enzymes can also be challenging due to the rapid degradation and denaturing of enzymes at ambient room temperature, as well as the enhanced degradation and denaturing that can occur with high temperature, pressure, humidity and/or exposure to light. Moisture and heat together can quickly destabilize enzymes, reducing their effectiveness, and shortening shelf life, leading to inaccurate dosing. Denaturation or destabilization of the enzymes can reduce their effectiveness by reducing the dose of active enzymes to less than the amount needed for effective treatment. Alternatively, attempting to compensate for the denaturation or destabilization by increasing the dose to ensure an effective level of active enzyme could risk an overdose or overfilling of a capsule or other dosage form. To protect and stabilize the pancreatic/digestive enzyme from unfavorable conditions, such a penetration, decomposition, the pancreatic/digestive enzyme (core) may be coated or encapsulated in a continuous coating containing an emulsifiable lipid. In another aspect, this invention provides new coated enzyme preparations with improved shelf life.

Manufacturers of enzyme preparations have used enteric coatings to deliver lipases in individuals requiring administration of lipases, such as individuals with cystic fibrosis. Because the porcine enzymes are delivered in a mixture of proteases, lipases and amylases, and because these compositions for human consumption were prepared for lipase delivery, the use of these enteric coatings, which include such substances as hypromellose phthalate, dimethicone 1000, and dibutyl phthalate, preclude delivery of proteases at the proper location in the digestive tract. All other enzyme preparations presently on the market contain at least one of these enteric coating substances and/or other additives in the preparation. Some additives that enable manufacturing, such as additives to improve flow properties, may further risk patient reactivity or sensitivity to the enzyme preparation.

In one embodiment the present invention includes a coated digestive enzyme preparation and/or composite, which, in some embodiments is an encapsulated pancreatic/digestive enzyme preparation. In other aspects, the invention includes enzyme delivery systems and pharmaceutical compositions comprising coated pancreatic/digestive enzyme preparations. These coated or encapsulated enzyme preparations contain cores comprising pancreatic or digestive enzyme particles, and a coating comprising an emulsifiable lipid.

The coatings in the digestive/pancreatic enzyme preparations create a barrier to degradation and denaturation and allow more accurate levels of active enzymes to reach the treated individuals. The lipid coating of this invention provides a significant barrier to moisture, heat, humidity and exposure to light by allowing for a physical barrier as well as one that prevents and/or reduces hydrolysis. The coated enzyme preparations undergo less hydrolysis as a result of protection from moisture in the environment by the lipid coating. As a result of the present invention, pancreatic/digestive enzymes are provided which can tolerate ranges of storage conditions (e.g., moisture, heat, oxygen, etc.) for long periods of time thus enabling extended shelf life. The coating of the encapsulated enzyme preparation protects the enzyme from the environment and provides emulsification in a solvent without detracting from the abrasion resistance of the coating. The invention thus further relates to more stable enzyme preparations.

It is another aspect of the present invention to make an enzyme preparation without the use of extenders, colorants, dyes, flow enhancers and other additives to reduce the potential for allergens and other sensitivity reactions in children and other treated individuals. It has been discovered that in some embodiments, the digestive enzymes can be encapsulated with a single lipid excipient to improve retention of enzyme activity, ease of administration, tolerability, and safety of administration, among other properties. Surprisingly, digestive enzyme particles containing lipases can be successfully encapsulated with coating consisting essentially of only hydrogenated soy oil.

In addition, porcine pancreatic/digestive enzymes possess a significant odor and taste, similar to cured/smoked pork. This taste can be strong and offensive to some individuals taking enzyme replacement, and especially to children. The addition of a lipid coating provides significant taste masking to the enzyme preparation, which allows for the tolerance of taste, as the lipid coating is odorless and tasteless. The use of this method of taste masking which does not involve the use of color, dyes, perfumes or other substances is preferable for the administration of medications, which have an unpleasant or undesirable taste and odor. In other embodiments, this invention relates to coated digestive enzyme preparations with improved taste and smell.

In addition, the encapsulation also provides controlled release of the pancreatic/digestive enzyme. The emulsification properties of the coating in a solvent allows for controlled release of the enzyme in the gastrointestinal system, preferably the region of the GI tract where the enzymes are to be utilized. The coating of the encapsulated composite protects the enzyme from the environment and provides emulsification in a solvent without detracting from the abrasion resistance of the coating. For example, for conditions requiring treatment with proteases, the release of the protease portion of the enzymes is necessary in the proximal small intestine, thereby necessitating a lipid encapsulation which has a dissolution profile between 30-90 minutes. The dissolution profile may also be about 30, 35, 40, 45, 50, 55, 60, 65, 70, 75, 80, 85 or 90 minutes. Dissolution profiles may be obtained using methods and conditions known to those of skill in the art. For example, dissolution profiles can be determined at various pHs, including pH 1, 2, 3, 4, 5, 6, 7, 8, 9, 10.

The rate of release of the bioactive substance can also be controlled by the addition of additives as described below. When the preparations are exposed to a solvent, the solvent interacts with the mollifiable lipid in the coating and results in emulsification of the coating and release of the bioactive substance.

“Encapsulate” as used herein means that the coating completely surrounds the pancreatic/digestive enzyme. In a population of encapsulated particles, encapsulated enzyme preparations may include improperly coated or small portion of particles with a substantially continuous coating as long as the release profiles of the encapsulated particles are not significantly altered. A coated or encapsulated particle may contain one or more digestive enzyme particles enveloped in one coating to form one coated or encapsulated digestive enzyme particle in the coated or encapsulated digestive enzyme preparation.

In some aspects, the invention relates to the production of selected coated enzyme preparations made by coating digestive enzyme particles with lipids not previously used in coated digestive enzyme preparations. The unique mixtures of emulsifiable lipids and enzymes can deliver certain components of the pancreatic/digestive enzymes to selected locations and/or at selected times during transit of the GI tract. In some aspects, the invention relates to methods of delivering digestive enzymes to humans based upon dissolution profiles.

The emulsifiable lipid is any lipid, lipid mixture, or blend of lipid and emulsifiers which emulsifies when exposed to a solvent and has a melting point which allows the lipid to be a solid at typical storage temperatures. The emulsifiable lipid can be a vegetable or animal derived-lipid. In some embodiments, the emulsifiable lipid consists essentially of, or comprises one or more monoglycerides, diglycerides or triglycerides, or other components including, for example, emulsifiers found in hydrogenated vegetable oils. In another embodiment the lipid is a non-polar lipid.

As used herein, animal and/or vegetable “derived” lipids can include fats and oils originating from plant or animal sources and/or tissues, and/or synthetically produced based on the structures of fats and oils originating from plant or animal sources. Lipid material may be refined, extracted or purified by known chemical or mechanical processes. Certain fatty acids present in lipids, termed essential fatty acids, must be present in the mammalian diet. The lipid may, in some embodiments, comprise a Type I U.S.P.-National Formulary vegetable oil.

The digestive enzyme used in the present invention can be any combination of digestive enzymes of a type produced by the pancreas, including, but not limited to digestive enzymes from a pancreatic source or other sources. The scope of the invention is not limited to pancreatic enzymes of porcine origin, but can be of other animal or plant origin as well as those which are synthetically derived. The digestive enzyme may be derived from mammalian sources such as porcine-derived digestive enzymes. The enzyme may include one or more enzymes, and can also be plant derived, synthetically derived, recombinantly produced in microbial, yeast, or mammalian cells, and can include a mixture of enzymes from one or more sources. Digestive enzyme can include, for example, one or more enzymes from one or more sources mixed together. This includes, for example, the addition of single digestive enzymes to digestive enzymes derived from pancreatic sources in order to provide appropriate levels of specific enzymes that provide more effective treatment for a selected disease or condition. One source of digestive enzymes can be obtained, for example, from Scientific Protein Laboratories. The digestive enzyme may be, for example a pancreatin/pancrealipase composition. In one embodiment, the digestive enzymes will comprise or consist essentially of 25 U.S.P. units/mg protease, 2 U.S.P. units/mg lipase, and 25 U.S.P. units/mg amylase. The term digestive enzyme may refer to one or more enzymes of a type produced by the pancreas.

The digestive enzyme particles used as cores in the present invention may include digestive enzyme particles where about 90% of the particles are between about #40 and #140 USSS mesh in size, or between about 105 to 425 μm, or where at least about 75% of the particles are between about #40 and #80 mesh, or about 180 to 425 μm in size. Particles between #40 and #140 mesh in size pass through #40 mesh but do not pass through #140 mesh. The coated or encapsulated digestive enzyme particles in one embodiment of this invention may comprise less than about 35, 30, 25, 20, 15 or 10% of the particles which can be sieved through #100 mesh (150 μm). In some embodiments, the term “non-aerosolizable” refers to a coated or encapsulated enzyme preparation where less than about 20% or less than about 15% of the particles can be sieved through #100 mesh (150 μm). The encapsulated digestive enzyme preparation can be an encapsulated digestive enzyme composite where the digestive enzyme particles contain two or more enzymes.

The particles may also be screened to obtain particles of a suitable size for encapsulation by removing particles that are too fine or too large. For example, the particles may be sieved to obtain particles of a suitable size or more uniform size range for encapsulation. As a further example, the particles may be sieved through USSS #40 mesh and through USSS #140 mesh. Particles that pass through the #40 mesh but are retained by the #140 mesh are of an appropriate size range for coating or encapsulation Particles may also be screened by sieving through USSS #140, #120, #100, #80, #70, #60, #50, #45, or #40 mesh, or any combination thereof. As used in connection with % particles that can be sieved, the term “about” can refer to plus or minus 10, 9, 8, 7, 6, 5, 4, 3, 2 or 1% or anywhere in-between.

The minimum amount of pancreatic enzyme present in the core is at least about 5% active enzymes by weight of the coated enzyme preparation, but in other embodiments may be at least about 30%, or at least about 50% by weight. The maximum amount of pancreatic/digestive enzyme present in the composite is at most about 95% by weight, and in other embodiments at most about 90%, 85%, 80%, 75% or 70% of the coated enzyme preparation. In other embodiments, the amount of pancreatic enzyme present in the composite is about 10%, 15%, 20%, 25%, 35%, 40%, 45%, 55%, 60%, 65%, 70%, 72.5%, 75%, 77.5%, 80%, 82.5%, 87.5%, or 92.5% by weight or anywhere in between. At least about or at most about a % of enzyme may include equal to or about that % of enzyme.

The composition which contains the encapsulated digestive enzyme preparation or composite can be delivered as a sprinkle, powder, capsule, tablet, pellet, caplet or other form. Packaging the encapsulated enzyme preparations in an enzyme delivery system that further comprises single dose sachet-housed sprinkle preparations allows for ease of delivery and accurate dosing of the enzyme, by allowing a specific amount of enzyme to be delivered in each dosing. Allowing for specific unit dosing of an enzyme preparation which maintains the enzyme activity within specific stability parameters is an enhancement over other sprinkle formulations, which are housed in a multi-unit dosing form that allows for air, moisture and heat to depredate and denature the enzyme preparation. In a preferred embodiment the powder or sachet is housed in a trilaminar foil pouch or similar barrier to keep out moisture and to protect the enzyme preparation from adverse environmental factors. The invention further relates to an improvement in stability due to a reduction in hydrolysis due to the lipid encapsulation.

Further the lipid encapsulation methodology reduces the aerosolization of the enzyme preparation that may be caustic to a child if inhaled through the lungs or the nose. In another embodiment, the invention includes delivery of digestive enzymes with improved safety of administration, by reducing the amount of aerosolization of the enzyme. The lipid encapsulation reduces aerosolization and the potential for caustic burn, aspiration, and/or aspiration pneumonias in children and administrators of the enzyme preparation, thereby reducing the potential for illness in already compromised children such as those with cystic fibrosis and leading to safer administration.

As used herein, the term “non-aerosolizable” will be used to refer to a coated or encapsulated enzyme preparation where substantially all of the particles are large enough to eliminate or reduce aerosolization upon pouring of the coated enzyme preparation compared to uncoated enzyme particles. For example, the term “non-aerosolizable” may refer to a coated or encapsulated enzyme preparation where at least about 90% of the particles are between about #40 and #140 mesh in size, or between about 106 to 425 μm, or where at least about 75% of the particles are between about #40 and #80 mesh, or about 180 to 425 μm. The term “non-aerosolizable” may also refer to a coated or encapsulated enzyme preparation where less than about 35, 30, 25, 20, 15 or 10% of the particles can be sieved through #100 mesh (150 μm). In some embodiments, the term “non-aerosolizable” refers to a coated or encapsulated enzyme preparation where less than about 20% or less than about 15% of the particles can be sieved through #100 mesh (150 μm).

As described and referred to herein, suitable pancreatic/digestive enzymes and suitable coatings may be used in the compositions and methods of this invention. The choice of suitable enzymes and of suitable lipid coatings, including choice of the type or amount of enzymes or coating, are guided by the specific enzyme needs of the individuals, and the selected diseases to be treated. The encapsulated enzyme preparations that are one aspect of this invention have not been previously described.

While general methods for coating certain sensitive biologic substances have been described, see, e.g., U.S. Pat. No. 6,251,478, hereby incorporated by reference, the encapsulated bioactive substance of this invention is an enzyme preparation comprising a core containing digestive enzymes comprising or consisting of multiple proteases, lipases and amylases, and a coating which comprises or consists essentially of an emulsifiable lipid.

Additives can be blended with the emulsifiable lipid. Selection of the lipid(s) and additives will control the rate of release of the bioactive substance. In the case of the digestive and or pancreatic enzymes, the lipid coat must be uniquely chosen to release the bioactive substance in the area of the digestive tract selected for release to optimize treatment.

Because in some embodiments the lipid encapsulation method does not require the enzyme preparation to be treated with solvents, extenders and excipients to facilitate flow or improve stability, one aspect of the invention includes a “clean” preparation of GRAS substances (generally regarded as safe) to be administered. The reduction in the use of solvents, extenders, excipients and other additives permitted by the methods of this invention reduces the exposure of the individuals taking the enzyme replacement to potential allergens, thereby producing a hypoallergenic enzyme preparation that further enhances its potential uses in the treatment of individuals who might otherwise develop an allergic response to treatment. Administration of the coated enzyme preparations of this invention can thus reduce exposure to potentially toxic substances and will also reduce the possibility of allergy formation. Accordingly, in some embodiments, the encapsulated digestive enzyme preparation is hypoallergenic.

In another embodiment, the invention relates to a method of controlling the rate of release of the pancreatic/digestive enzyme from an encapsulated enzyme preparation upon exposure to a solvent. In some aspects, the method comprises blending an emulsifiable lipid with an amount of one or more additives to obtain a lipid blend; and coating the digestive enzyme particle with the blend to form an encapsulated digestive enzyme preparation containing particles comprising a core which contains the enzyme, and a coating which contains the lipid. In some embodiments, the emulsifiable lipid is a blend where the emulsifiable lipid and additive are not the same, and where the rate of release of the enzyme from the encapsulated composite upon exposure to a solvent is decreased as the amount of additive is increased. In the alternative, the rate of release of the enzyme from the encapsulated composite upon exposure to a solvent is increased as the amount of additive is decreased.

The lipid coating surprisingly does not appear to be reduced or destroyed by hydrochloric acid (HCl) present in the stomach, thereby protecting the enzyme from degradation following administration until the enzyme preparation reaches its target region in the GI tract. Further the lipid coat reduces the exposure of the enzyme to attack by water, thereby reducing hydrolysis, and further protecting the digestive enzymes from degradation. In addition, the inventors have found that an excipient containing only lipid can be used to coat or encapsulate digestive enzyme particles containing lipase.

Enzyme preparations supplied by the API supplier may be provided as irregularly shaped, multi-sized particles with uneven edges, and much clumping, and containing some crystalline salt particles. Uneven particle size and shape reduces flow properties and interferes with packaging. In addition, pouring uncoated enzyme into the mouth of a subject would be difficult, and potentially may cause too much or too little of the enzyme to be delivered. Processing the digestive enzyme particles according to methods in accordance with one aspect of this invention yields a non-dusty, free-flowing particulate preparation suitable for sachet packaging and for pouring onto food or drink. In addition, as discussed throughout, the use of lipid encapsulation to prevent aerosolization, (and therefore increase safety) and to increase flow properties, which enhance manufacturing of a pharmaceutical, is an embodiment of the instant invention.

The size distribution of particles in an exemplary raw enzyme preparation is shown in the graph in FIG. 3. Large particles (>20 mesh) and very small particles (<140 mesh) are generally not suitable for proper encapsulation and can be removed by screening. In order to increase the flow properties of the encapsulated pancreatic enzyme preparation, digestive enzyme particles can be sieved to remove fines and overly large particles, for example by including only particles of sizes obtainable by using screens of USSS 20-140 mesh, or about 105 to 841 microns. In some embodiments, the coated digestive enzyme preparation containing 80% digestive enzyme by weight is made by coating sieved pancreatic enzyme particles with a hydrogenated vegetable oil using 20 lbs. of enzyme particles and 5 lbs of hydrogenated vegetable oil. In some embodiments, the temperature of the lipid or lipid blend is maintained at 110° F. before application to the digestive enzymes, which are not heated.

In some embodiments, the lipid should be present in the preparation at a minimum amount of about 5% by weight of the encapsulated composite, preferably about 30%, and more preferably about 50% by weight of the encapsulated composite. The maximum amount of pancreatic/digestive enzyme present in the encapsulated composite is about 95% by weight of the composite, preferably about 90%, and more preferably about 85% of the encapsulated composite. The emulsifiable lipid can be any lipid or lipid-derived material that emulsifies or creates an emulsion yet has a melting point which allows the emulsifiable lipid to be a solid at typical storage temperatures, for example, 23 degrees Centigrade.

“Emulsifiable lipids” as used herein means those lipids which contain at least one hydrophilic group and at least one hydrophobic group and have a structure capable of forming a hydrophilic and hydrophobic interface. These chemical and/or physical properties, mentioned above, of an emulsifiable lipid permit emulsification. Examples of interfaces include, for example, micelles and bilayers. The hydrophilic group can be a polar group and can be charged or uncharged.

The emulsifiable lipid can be derived from animal or vegetable origins, such as, for example, palm kernel oil, soybean oil, cottonseed oil, canola oil, and poultry fat, including hydrogenated type I vegetable oils. In some embodiments, the lipid is hydrogenated. The lipid can also be saturated or partially saturated. Examples of emulsifiable lipids include, but are not limited to, monoglycerides, diglycerides, fatty acids, esters of fatty acids, phospholipids, salts thereof, and combinations thereof.

The emulsifiable lipid is preferably a food grade emulsifiable lipid. Some examples of food grade emulsifiable lipids include sorbitan monostearates, sorbitan tristearates, calcium stearoyl lactylates, and calcium stearoyl lactylates. Examples of food grade fatty acid esters which are emulsifiable lipids include acetic acid esters of mono- and diglycerides, citric acid esters of mono- and diglycerides, lactic acid esters of mono- and diglycerides, polyglycerol esters of fatty acids, propylene glycol esters of fatty acids, and diacetyl tartaric acid esters of mono- and diglycerides. Lipids can include, for example, hydrogenated soy oil. Any emulsifiable lipid may be used in the methods and products of this invention. In certain embodiments the emulsifiable lipid used will produce non-agglomerating, non-aerosolizing enzyme preparation particles.

In other embodiments, the method relates to preparation of an encapsulated, controlled release digestive enzyme preparation with enhanced flow properties useful in the treatment of individuals with Celiac disease, the method comprising: a) blending an emulsifiable lipid with one or more additives to obtain a blend; and b) coating screened digestive enzyme with the blend to form an encapsulated digestive enzyme containing a core which contains the digestive enzyme and a coating which contains the blend of emulsifiable lipid.

The coating of the enzyme with the lipid allows for the enzyme to become more uniform in size and shape. reduces the jagged edges associated with the raw enzyme and allows for ease of administration and ease of manufacturing, as the flow properties associated with the covered enzyme will allow for the manufacturing machinery to easily fill the sachet/pouch with the enzyme and reduces overfilling or underfilling of the sachet. The unit dose packaging reduces the ability of a child to open the multidose can/box/or other container. The trilaminar foil pouch or sachet further reduces the ability of a child to open the sachet/pouch, prohibiting possible overdose.

In another embodiment, the invention relates to a method of controlling the rate of release of a digestive enzyme from the encapsulated preparation by using a lipid blend to coat the digestive enzyme. The method includes blending an emulsifiable lipid with one or more additives to obtain a blend, and coating the digestive enzyme with the blend to form an encapsulated digestive enzyme containing a core which contains the digestive enzyme and a coating which contains the blend of emulsifiable lipid. The rate of release of the enzyme from the encapsulated preparation upon exposure with a solvent is decreased as the amount of additive is increased. In the alternative, the rate of release of the enzyme from the encapsulated composite upon exposure with a solvent is increased as the amount of additive is decreased. Thus, the nature of the coating allows for controlled release of the enzyme from the encapsulate.

Non-emulsifiable lipids do not possess the chemical and/or physical properties related to emulsification as described above and include any lipid, lipid derived material, waxes, organic esters, or combinations thereof. Non-emulsifiable lipids generally do not emulsify by themselves. Non-emulsifiable lipids can be used as additives so long as the properties of the coating, and constituent lipids, permit emulsification. Non-emulsifiable lipids, such as, for example, triglycerides, can be blended with an emulsifiable lipid of the present invention. The non-emulsifiable lipid can be derived from animals, vegetables, mineral, or synthetic origins. The non-emulsifiable lipid is preferably hydrogenated, and can be saturated or partially saturated, and includes, but is not limited to triglycerides. In a preferred embodiment, the coating contains a blend of monoglycerides and triglycerides applied to a pancreatic/digestive enzyme.

The inclusion of one or more additives with an emulsifiable lipid of the present invention is used to control emulsification of the coating and release of the enzyme. For example, the additive, triglyceride, can be blended with monoglycerides (e.g., an emulsifiable lipid), to control emulsification of the coating and thus control (e.g., decrease) the rate of release of the enzyme from the composite. As a further example, one or more additives, such as a diglyceride and a triglyceride can be blended with the emulsifiable lipid to control the rate of release of the enzyme. Hydrogenated vegetable oils may contain emulsifying agents, such as soy lecithin or other components.

Properties including mechanical strength, melting point and hydrophobicity can be considered when choosing a suitable lipid coating for the digestive enzyme. Lipids having lower melting points or more polar, hydrophilic properties were generally less suitable for encapsulation because they resulted in product that would cake under accelerated storage stability conditions. Enzyme preparations made using, for example, hydrogenated soy oil, hydrogenated castor wax, and carnauba wax all demonstrated good pouring and no caking.

The wax can be paraffin wax; a petroleum wax; a mineral wax such as ozokerite, ceresin, or montan wax; a vegetable wax such as, for example, carnauba wax, bayberry wax, or flax wax; an animal wax such as, for example, spermaceti; or an insect wax such as beeswax.

Additionally, the wax material can be an ester of a fatty acid having 12 to 31 carbon atoms and a fatty alcohol having 12 to 31 carbon atoms, the ester having from a carbon atom content of from 24 to 62, or a mixture thereof. Examples include myricyl palmitate, cetyl palmitate, myricyl cerotate, cetyl myristate, ceryl palmitate, ceryl certate, myricyl melissate, stearyl palmitate, stearyl myristate, and lauryl laurate.

In a further embodiment, the invention provides a method for controlling rate of release of a pancreatic/digestive enzyme from an encapsulated composite upon exposure to a solvent. The method includes coating the enzyme with an amount of an emulsifiable lipid to form an encapsulated pancreatic enzyme substance composite, wherein the rate of release of the enzyme from the encapsulated composite is decreased as the amount of emulsifiable lipid based on total weight of the encapsulated composite is increased. In the alternative, the rate of release of the pancreatic enzyme from the encapsulated composite is increased as the amount of emulsifiable lipid based on total weight of the encapsulated composite is decreased. The emulsifiable lipid useful in this embodiment can consists essentially of one or more monoglycerides.

The solvent in which a lipid emulsifies can be an aqueous solvent. The aqueous solvent interacts with the hydrophilic groups present in the emulsifiable lipid and disrupts the continuity of the coating, resulting in an emulsion between the aqueous solvent and the lipids in the coating, thus releasing the bioactive substance from the composites.

Compositions described herein may also be formulated for administration as a suppository. For example, a gel formulated into a suppository would be one preferred product form for administration of digestive enzymes to mucosal surfaces of either the rectum or the vagina. Methods of making suppositories are known in the art and contemplated herein.

The phrase “pharmaceutically acceptable” refers to molecular entities and compositions that are physiologically tolerable and do not typically produce an allergic or similar untoward reaction, such as gastric upset, dizziness and the like, when administered to a human.

### Dosages and Methods of Treatment

Methods of preparing dosage forms are known, or will be apparent, to those skilled in this art; for example, see Remington: The Science and Practice of Pharmacy, 21st Edition (Lippincott Williams & Wilkins. 2005). Appropriate dosages will depend on the patient (age, weight, overall health, etc.), the severity of the condition, the type of formulation and other factors known to those having ordinary skill in the art. It is to be noted that concentrations and dosage values can vary with the severity of the condition. It is to be further understood that for any particular patient, specific dosage regimens should be adjusted over time according to the individual need and the professional judgment of the person administering or supervising the administration of the compositions.

The invention also relates to a specific blend of enzymes, with or without coating, with or without other components as described above whereby enzyme administration occurs in individuals with Celiac disease and related disorders, including but not limited to: Celiac disease, celiac sprue, non-tropical sprue, gluten-sensitive enteropathy and irritable bowel syndrome.

Provided herein is a method for alleviating symptoms of Celiac disease and related conditions including also known as sprue, non-tropical sprue, gluten intolerance or gluten-sensitive enteropathy. The method comprises the administration to a subject of a digestive enzyme either naturally or recombinantly derived, or their derivatives in an amount effective to reduce the symptoms of celiac and related conditions.

In one aspect, provided herein is a method for treating a subject exhibiting one or more symptoms of Celiac disease and related disorders, the method comprising administering a therapeutically effective amount of digestive enzymes to the individual. In one embodiment, the disorders comprise: Celiac disease, non-tropical sprue and celiac sprue.

In another embodiment, the symptoms of Celiac disease and other related disorders which are potentially suited for alleviation according to the present method are selected from the group consisting of gastrointestinal symptoms and include but are not limited to: irritable bowel syndrome, protein deficiency, iron-deficiency anemia caused by menstrual blood loss or protein deficiency, inflammatory bowel disease, diverticulitis, intestinal infections, stool abnormalities, diarrhea which may be protracted or intermittent, constipation, alternating constipation with diarrhea, abdominal pain, bloating, gas or indigestion, decreased appetite (may also be increased or unchanged), lactose intolerance, nausea and vomiting, stools that float, are foul smelling, bloody, or “fatty”, and unexplained weight loss.

In another embodiment, the symptoms of celiac and related disorders which are potentially suited for alleviation according to the present method are selected from those which represent neurological or neuropsychiatric symptoms and include but are not limited to: depression, anxiety, chronic headaches, migraines, fatigue, memory loss, dementia, mania, hypomania, malaise (which may be protracted) seizures, tingling or numbness especially in the extremities and the head, seizures, and irritable and fussy behavior in children.

In another embodiment, the symptoms of celiac and related disorders which are potentially suited for alleviation according to the present method are selected from those which represent metabolic, dermatological, immunological or hormonal or symptoms and include but are not limited to: bruising easily, hair loss, mouth ulcers, nosebleeds, missed menstrual periods, Vitamin B12 deficiency, vitamin deficiencies, mineral deficiencies, osteoporosis, delayed growth in children, itchy skin (dermatitis herpetiformis), muscle cramps, joint pain, short stature, poor weight gain, slowed growth, failure to thrive, low cholesterol, pancreatic cancer, lymphoma, gastric cancer, colon cancer, and intestinal cancer.

Individuals with Celiac disease and other associated disorders generally exhibit one or more symptoms of the disease or disorder. In addition, they often exhibit symptoms of co-morbid conditions such as thyroid disease, autoimmune disorders such as Rheumatoid Arthritis, Systemic Lupus Erythematosus, and Sjogren Syndrome, as well as Addison's Disease, Down Syndrome, Turner Syndrome, lactose intolerance and Type 1 Diabetes.

Recognition and determination of a reduction in symptoms of Celiac disease, related disorders and resultant symptomatology can be performed by those skilled in the art. The skilled artisan will recognize that those who suffer from the related disorders can potentially have Celiac disease.

The recognition of the symptoms of celiac and related disorders present in a subject and determination that the present method may alleviate said symptoms prior to, during, or after the practice of this method is well within the purview of a subject ordinarily skilled in the art, who can perform suitable clinical, diagnostic, and/or observational or other techniques required.

Another aspect of the invention relates to the method for treating Celiac disease and related disorders in a subject comprising administering an effective amount of a composition comprising one or more digestive enzymes to the individual. In one embodiment the symptoms of Celiac disease and related disorders are selected from a group comprising gastrointestinal symptoms and include, but are not limited to, irritable bowel syndrome, protein deficiency, iron-deficiency anemia caused by menstrual blood loss or protein deficiency, inflammatory bowel disease, diverticulitis, intestinal infections, stool abnormalities, diarrhea which may be protracted or intermittent, constipation, alternating constipation with diarrhea, abdominal pain, bloating, gas or indigestion, decreased appetite (may also be increased or unchanged), lactose intolerance, nausea and vomiting, stools that float, are foul smelling, bloody, or “fatty”, and unexplained weight loss. In yet another embodiment the symptoms comprise those which are neurological or neuropsychiatric symptoms and include but are not limited to: depression, anxiety, chronic headaches, migraines, fatigue, memory loss, dementia, mania, hypomania, malaise (which may be protracted) seizures, tingling or numbness especially in the extremities and the head, seizures and irritable and fussy behavior in children. In another embodiment, the symptoms are metabolic, dermatological, immunological or hormonal and include, but are not limited to, bruising easily, hair loss, mouth ulcers, nosebleeds, missed menstrual periods, low cholesterol, Vitamin B12 deficiency, vitamin deficiencies, mineral deficiencies, osteoporosis, delayed growth in children, itchy skin (dermatitis herpetiformis), muscle cramps, joint pain, short stature, poor weight gain, slowed growth, failure to thrive, lymphoma, gastric cancer, colon cancer, intestinal cancer, and pancreatic cancer.

The invention further relates to methods for administering the enzyme preparations. In some aspects, the methods include administering the pancreatic/digestive enzymes as coated preparations. In some aspects, the invention relates to a method of treatment comprising administering to a subject with Celiac disease or related disorders, including but not limited to: Celiac disease, celiac sprue, non-tropical sprue, and gluten-sensitive enteropathy, irritable bowel syndrome, and chronic fatigue, in need of treatment with digestive enzymes, at least two doses of a composition comprising a therapeutically effective amount of a coated or uncoated digestive enzyme preparation comprising a core comprising a digestive enzyme; and in the case of a coated enzyme preparation, a coating comprising an emulsifiable lipid. Determination of whether a subject is in need of treatment with an effective amount of digestive enzymes may be based on a determination that the subject has an enzyme deficiency.

In yet another aspect, the methods include administering the pancreatic/digestive enzymes as coated preparations. In some aspects, the invention relates to a method of treatment comprising administering to a subject with Celiac disease or related disorders, including but not limited to those who exhibit gastrointestinal symptoms which include but also are not limited to: irritable bowel syndrome, protein deficiency, iron-deficiency anemia caused by menstrual blood loss or protein deficiency, inflammatory bowel disease, diverticulitis, intestinal infections, stool abnormalities, diarrhea which may be protracted or intermittent, constipation, alternating constipation with diarrhea, abdominal pain, bloating, gas or indigestion, decreased appetite (may also be increased or unchanged), lactose intolerance, nausea and vomiting, stools that float, are foul smelling, bloody, or “fatty”, and unexplained weight loss; in need of treatment with digestive enzymes, at least two doses of a composition comprising a therapeutically effective amount of a coated or uncoated digestive enzyme preparation comprising a core comprising a digestive enzyme; and in the case of a coated enzyme preparation, a coating comprising an emulsifiable lipid. Determination of whether a subject is in need of treatment with an effective amount of digestive enzymes may be based on a determination that the subject has an enzyme deficiency.

In yet another aspect, the methods include administering the pancreatic/digestive enzymes as coated preparations. In some aspects, the invention relates to a method of treatment comprising administering to a subject with Celiac disease or related disorders, including but not limited to those who exhibit neurological or neuropsychiatric symptoms and include but are not limited to: depression, anxiety, chronic headaches, migraines, fatigue, memory loss, dementia, mania, hypomania, malaise (which may be protracted) seizures, tingling or numbness especially in the extremities and the head, seizures and irritable and fussy behavior in children, in need of treatment with digestive enzymes, at least two doses of a composition comprising a therapeutically effective amount of a coated or uncoated digestive enzyme preparation comprising a core comprising a digestive enzyme; and in the case of a coated enzyme preparation, a coating comprising an emulsifiable lipid. Determination of whether a subject is in need of treatment with an effective amount of digestive enzymes may be based on a determination that the subject has an enzyme deficiency.

In yet another aspect, the methods include administering the pancreatic/digestive enzymes as coated preparations. In some aspects, the invention relates to a method of treatment comprising administering to a subject with Celiac disease or related disorders, including but not limited to those who exhibit metabolic, dermatological, immunological or hormonal or symptoms and include but are not limited to: bruising easily, hair loss, mouth ulcers, nosebleeds, missed menstrual periods, low cholesterol, Vitamin B12 deficiency, vitamin deficiencies, mineral deficiencies, osteoporosis, delayed growth in children, itchy skin (dermatitis herpetiformis), muscle cramps, joint pain, short stature, poor weight gain, slowed growth, failure to thrive, lymphoma, gastric cancer, colon cancer, intestinal cancer and pancreatic cancer, in need of treatment with digestive enzymes, at least two doses of a composition comprising a therapeutically effective amount of a coated or uncoated digestive enzyme preparation comprising a core comprising a digestive enzyme; and in the case of a coated enzyme preparation, a coating comprising an emulsifiable lipid. Determination of whether a subject is in need of treatment with an effective amount of digestive enzymes may be based on a determination that the subject has an enzyme deficiency.

In another aspect of the invention, it is well known that determining a dosage regimen of the compound is well within the purview of those skilled in the art. By way of example, the dose levels may range from 900 milligrams to 10 grams as determined by weight. Further activity of the enzymes may range from 100 units of activity to 1,000,000 units of activity per dose for amylases, lipases and proteases.

In some embodiments, each dose contains about 100 to 1500 mg of coated or encapsulated enzyme preparation, and each dose may contain about 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, or 1500 mg of coated or encapsulated enzyme preparation. “About” can include 80 to 125% of the recited preparation. Each dose may also be plus or minus 2%, 5%, or 10% of the recited weight. In one embodiment each does will have a protease activity of not less than about 156 U.S.P. units/dose±2%, 5%, or 10%. The protease activity may also be not less than about 100, 105, 110, 115, 120, 125, 130, 135, 140, 145, 150, 155, 160, 165, 170, 175, 180, 185, 190, 195, or 200 U.S.P. units/dose.

In another embodiment, the invention relates to methods of treatment comprising administering to a subject with a neurological or neuropsychiatric condition susceptible to treatment with digestive enzymes, at least two doses of a composition comprising a therapeutically effective amount of the coated digestive enzyme preparations. In certain embodiments, about 80% of the enzyme is released by about 30 minutes in a dissolution test performed at pH 6.0. In other embodiments, about 80% of the enzyme is released by about 30 minutes after the coated digestive enzyme preparations reach the small intestine.

The invention further relates in another aspect to the delivery of digestive enzymes with improved safety of administration. The lipid coat adds weight to the enzyme preparation, which reduces the potential for aerosolization. Previous uncoated enzymes have been shown to become aerosolized, and can therefore be inhaled and contact the nasal cavity or the lungs, causing injury to the mucosa of those taking and those administering the enzyme preparation.

The invention further relates to the improvement of administering a sachet preparation for delivery to children. The invention specifically relates to the administration of a coated digestive enzyme preparation, housed in a sachet which allows for particular types of administration including but not limited to administration in food, drink, or direct administration into the oral cavity or directly into the GI system through a NG-tube, G-tube or other GI entrances. The use of a sachet delivery of enzymes has heretofore been not utilized in the enzyme preparations presently marketed. In one embodiment, the sachet represents a single unit dosage or multiple doses for a day. The sachet of a trilaminar foil allows the enzyme/lipid powder to remain stable, and allows for ease of administration.

The methods further relate to the administering of the coated and/or encapsulated enzyme preparation in a sachet or pouch preparation for ease of delivery to children and adults. In some embodiments, the invention specifically relates to the administration of a coated enzyme particle preparation, housed in a sachet or pouch. This facilitates administration, including but not limited to, administration in food or drink, direct administration into the oral cavity, or administration directly into the GI system through an NG-tube, G-tube or other GI entrances or deliveries.

Compositions comprising an effective amount of the compound may be administered via any conventional route including but not limited to oral, parenteral, intramuscular, intravenous, transmucosal, transdermal, via suppository or other method. Further the oral administration can be in the form of pellets, capsules, caplets, beadlets, sprinkles, tablets, softgels, or other carrier.

The pharmaceutical formulations can also be prepared for parenteral use. Such formulations typically take the form of sterile isotonic solutions of the active ingredient according to standard pharmaceutical practice.

In one embodiment of the present invention, the increase of protein digestion of a subject suffering from Celiac disease or other related disorders leads to the improvement of such disorders. In another embodiment, a subject suffering from or diagnosed with Celiac disease or related disorders benefits from the administration of digestive enzymes since digestive enzymes aid in the protein digestion process. In one embodiment, the Celiac disease or related disorder symptoms of a subject suffering from or diagnosed with celiac or related disorders is improved or alleviated from the administration of digestive enzymes.

The present invention provides a method for using digestive enzymes and their derivatives to alleviate the symptoms of celiac or related disorders. The method comprises administering to the individual a digestive enzyme either naturally or recombinantly derived, or their derivatives, in an amount effective to reduce the symptoms of Celiac disease or related disorders.

In another embodiment, the administration of digestive enzymes to those with celiac or related disorders who exhibit symptoms of said disease or disorders is utilized to examine the efficacy of the digestive enzyme preparation with respect to administration of an effective amount of the enzyme and evaluating the enzyme's effect on the reduction of symptomatology associated with Celiac disease or related disorders.

The application of these enzymes of the high protease classification as applied to individuals with Celiac disease or related disorders represents a novel discovery for the use of digestive enzymes.

In one embodiment, a composition can be administered 1 or more times a day, such as 1, 2, 3, 4, 5, 6, 7, 8, 9, or 10 times a day with or without food. In another embodiment, a composition can be administered orally 3 times a day with or without food.

The term “unit dose” when used in reference to a therapeutic composition refers to physically discrete units suitable as unitary dosage for humans, each unit containing a predetermined quantity of active material calculated to produce the desired therapeutic effect in association with the required diluent; i.e., carrier, or vehicle.

Provided herein are methods for administering the enzyme compositions/preparations. In one aspect, the methods include administering the pancreatic/digestive enzymes as coated preparations. In another aspect, the disclosure relates to a method of treatment comprising administering to a subject with Celiac disease or a related disorder, including but not limited to: bruising easily, hair loss, mouth ulcers, nosebleeds, missed menstrual periods, Vitamin B12 deficiency, vitamin deficiencies, mineral deficiencies, osteoporosis, delayed growth in children, itchy skin (dermatitis herpetiformis), muscle cramps, joint pain, short stature, poor weight gain, slowed growth, failure to thrive, low cholesterol, pancreatic cancer, lymphoma, gastric cancer, colon cancer, intestinal cancer, autoimmune disorders such as rheumatoid arthritis, systemic lupus erythematosus, and Sjogren syndrome; Addison's disease; Down syndrome; Intestinal cancer; Intestinal lymphoma; Lactose intolerance; Thyroid disease; and/or Type 1 diabetes. The method comprises administering to the individual a digestive enzyme either naturally or recombinantly derived, or their derivatives, in an amount effective to reduce the symptoms of celiac and related disorders. The enzymes may be coated or uncoated.

The present invention provides a method for using digestive enzymes and their derivatives to alleviate one or more symptoms of Celiac disease or a related disorder. Symptoms to be alleviated include, but are not limited to, gastrointestinal symptoms and include, but are not limited to, irritable bowel syndrome, protein deficiency, iron-deficiency anemia caused by menstrual blood loss or protein deficiency, inflammatory bowel disease, diverticulitis, intestinal infections, stool abnormalities, diarrhea which may be protracted or intermittent, constipation, alternating constipation with diarrhea, abdominal pain, bloating, gas or indigestion, decreased appetite (may also be increased or unchanged), lactose intolerance, nausea and vomiting, stools that float, are foul smelling, bloody, or “fatty”, and unexplained weight loss.

In one aspect of the present disclosure, it is well known that determining a dosage regimen of the compound is well within the purview of those in the art. By way of example, the dose levels may range from 100 milligrams to 10 grams as determined by weight. Further activity of the enzymes may range from 100 units of activity to 1,500,000 units of activity per dose for amylases, lipases and proteases, respectively.

In another embodiment, the disclosure relates to methods of treatment comprising administering to a subject with a neurological or neuropsychiatric condition susceptible to treatment with digestive enzymes, at least two doses of a composition comprising a therapeutically effective amount of the coated digestive enzyme preparations. In certain embodiments, about 80% of the enzyme is released by about 30 minutes in a dissolution test performed at pH 6.0. In other embodiments, about 80% of the enzyme is released by about 30 minutes after the coated digestive enzyme preparations reach the small intestine.

The disclosure further relates in another aspect to the delivery of digestive enzymes with improved safety of administration. The lipid coat adds weight to the enzyme preparation, which reduces the potential for aerosolization. Previous uncoated enzymes have been shown to become aerosolized, and can therefore be inhaled and contact the nasal cavity or the lungs, causing injury to the mucosa of those taking and those administering the enzyme preparation.

The disclosure further relates to the improvement of administering a sachet preparation for delivery to children. The disclosure specifically relates to the administration of a coated or uncoated digestive enzyme preparation, housed in a sachet which allows for particular types of administration including but not limited to administration in food, drink, or direct administration into the oral cavity or directly into the GI system through a NG-tube, G-tube or other GI entrances. The use of a sachet delivery of enzymes has heretofore not been utilized in the enzyme preparations presently marketed. In one embodiment, the sachet represents a single unit dosage or multiple doses for a day. The sachet of a trilaminar pouch allows the enzyme or enzyme/lipid powder to remain stable, and allows for ease of administration.

The disclosure further relates to the administering of the coated or uncoated enzyme preparation in a sachet or pouch preparation for ease of delivery to children and adults. In some embodiments, the disclosure specifically relates to the administration of a coated or uncoated enzyme particle preparation, housed in a sachet or pouch. This facilitates administration, including but not limited to, administration in food or drink, direct administration into the oral cavity, or administration directly into the GI system through an NG-tube, G-tube or other GI entrances or deliveries.

Compositions comprising an effective amount of the compound may be administered via any conventional route including but not limited to oral, parenteral, intramuscular, intravenous, transmucosal, transdermal, suppository or other method. Further the oral administration can be in the form of pellets, capsules, caplets, beadlets, sprinkles, tablets, softgels, or other carrier.

In one embodiment, the increase of protein digestion of a subject suffering from Celiac disease or a related disorder to the improvement of such disease or disorders. In another embodiment, a subject suffering from or diagnosed with Celiac disease benefits from the administration of digestive enzymes. In one embodiment, the neuropsychiatric symptoms of a subject suffering from or diagnosed with Celiac disease is improved or alleviated from the administration of digestive enzymes.

The present invention provides a method for using digestive enzymes and their derivatives to alleviate the symptoms of Celiac disease. The method comprises administering to the individual a digestive enzyme either naturally or recombinantly derived, or their derivatives, in an amount effective to reduce one or more symptoms of Celiac disease.

Provided herein are methods of preventing one or more symptoms associated with Celiac disease by administering a composition described herein. As used herein, “prevention” refers to prophylaxis, prevention of onset of symptoms, prevention of progression of a Celiac disease. As used herein, “inhibition”, “prevention”, “treatment” and “treating” refer to, for example, stasis of symptoms, as well as partial or full amelioration of one or more symptoms associated with Celiac disease. Because Celiac disease is hereditary, family members of a person with the disease may wish to be tested. Such patients can be monitored and be treated as needed with a composition described herein.

Compositions can be administered to a patient in an amount that is effective for producing some desired therapeutic effect by alleviating one or more symptoms associated with Celiac disease at a reasonable benefit/risk ratio applicable to any medical treatment. A therapeutically effective amount is an amount achieves at least partially a desired therapeutic or prophylactic effect in tissue subject. The amount of digestive enzymes necessary to bring about alleviation one or more symptoms associated with Celiac disease is not fixed per se. The amount of digestive enzymes administered may vary with the type of disorder, extensiveness of the disorder, and size of the patient suffering from the disorder. A response is achieved when the patient experiences partial or total alleviation, or reduction of one or more signs or symptoms of illness. The patient's symptoms can remain static (i.e., not get worse) or can be reduced.

A physician can readily determine and prescribe the effective amount (ED50) of the composition required. For example, the physician could start doses of the compounds employed in the composition at levels lower than that required in order to achieve the desired therapeutic effect and gradually increase the dosage until the desired effect is achieved. Alternatively, a dose can remain constant.

In such methods of treatment, one or more symptoms are ameliorated or reduced following administration of a composition provided herein. In one embodiment, one or more symptoms of such disorders are reduced in severity or duration by about 2%, about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100%. In another embodiment, one or more symptoms of such disorders are reduced in severity or duration by about 2-fold, about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 90-fold, about 95-fold, about 100-fold or more.

The diagnostic criteria described above may be used to assess whether administration of a composition described herein reduces the severity and/or duration of one or more symptoms of Celiac disease or a related disorder.

In yet another embodiment, the duration of symptoms may be reduced in severity and/or duration following administration of a composition described herein. That is, one or more symptoms may persist for less than 6 months, 5 months, 4 months, 3 months, 2 months, 1 month, 3 weeks, 2 weeks or 1 week.

### Combination Therapy

Another aspect provided herein is combination therapy of a patient with a composition described herein along with another therapeutically effective agent or rehabilitation. Such treatments include, but are not limited to, administration of monoclonal-, or polyclonal-, monomeric, dimeric, or polymeric IgA; peptide inhibitors of gliadin peptides; gluten-free diet.

Treatment may also be combined with an adherence to a lifelong gluten-free diet, thereby allowing the intestinal villi to heal. Foods, beverages, and medications that contain wheat, barley, and rye are eliminated. Wheat, rye, and barley are the grains that contain pathogenic peptides. Gastrointestinal symptoms in patients with symptomatic Celiac disease who adhere to a gluten-free diet typically resolve within a few weeks.

A gluten-free diet means not eating foods that contain wheat, rye and barley. The foods and products made from these grains should also be avoided. In other words, a person with Celiac disease should not eat most grain, pasta, cereal and many processed foods.

Despite these restrictions, people with Celiac disease can eat a well-balanced diet with a variety of foods. They can use potato, rice, soy, amaranth, quinoa, buckwheat, or bean flour instead of wheat flour. They can buy gluten-free bread, pasta, and other products from stores that carry organic foods, or order products from special food companies. “Plain” meat, fish, rice, fruits, and vegetables do not contain gluten, so people with Celiac disease can freely eat these foods. In the past, people with Celiac disease were advised not to eat oats. New evidence suggests that most people can safely eat small amounts of oats, as long as the oats are not contaminated with wheat gluten during processing.

People with Celiac disease should work closely with their health care team when deciding whether to include oats in their diet. The gluten-free diet requires a completely new approach to eating. Newly diagnosed people and their families may find support groups helpful as they learn to adjust to a new way of life. People with Celiac disease must be cautious about what they buy for lunch at school or work, what they purchase at the grocery store, what they eat at restaurants or parties, and what they grab for a snack. Eating out can be a challenge. When in doubt about a menu item, a person with Celiac disease should ask the waiter or chef about ingredients and preparation or if a gluten-free menu is available.

Gluten is also used in some medications. People with Celiac disease should ask a pharmacist if prescribed medications contain wheat. Because gluten is sometimes used as an additive in unexpected products-such as lipstick and Play Dough-reading product labels is important. If the ingredients are not listed on the label, the manufacturer should provide a list upon request. With practice, screening for gluten becomes second nature.

In addition to meats and vegetable and gluten free dairy foods, the following are allowable foods for those with gluten sensitivity/Celiac disease: Acacia Gum; Acesulfame K; Acesulfame Potassium; Acetanisole; Acetophenone; Acorn Quercus; Adipic Acid; Adzuki Bean; Acacia Gum; Agar; Agave; Albumen; Alcohol (Spirits—Specific Types); Alfalfa; Algae; Algin; Alginic Acid; Alginate; Alkalized Cocoa; Allicin; Almond Nut; Alpha-amylase; Alpha-lactalbumin; Aluminum; Amaranth; Ambergris; Ammonium Hydroxide; Ammonium Phosphate; Ammonium Sulphate; Amylose; Amylopectin; Annatto; Annatto Color; Apple Cider Vinegar; Arabic Gum; Arrowroot; Artichokes; Artificial Butter Flavor; Artificial Flavoring; Ascorbic Acid; Aspartame (can cause IBS symptoms); Aspartic Acid; Aspic; Astragalus Gummifer; Autolyzed Yeast Extract; Avena Sativia; Avena Sativia Extract; Avidin; Azodicarbonamide; baking soda; Balsamic Vinegar; Beeswax; Beans; Bean, Adzuki; Bean, Hyacinth; Bean, Lentil; Bean, Mung; Bean Romano (Chickpea); Bean Tepary; Benzoic acid; Besan (Chickpea); Beta Glucan; Betaine; Beta Carotene; BHA; BHT; Bicarbonate of Soda; Biotin; Blue Cheese; Brown Sugar; Buckwheat; Butter (check additives); Butylated Hydroxyanisole; Butyl Compounds; Calcium Acetate; Calcium Carbonate; Calcium Caseinate; Calcium Chloride; Calcium Disodium; Calcium Hydroxide; Calcium Lactate; Calcium Pantothenate; Calcium Phosphate; Calcium Propionate; Calcium Silicate; Calcium Sorbate; Calcium Stearoyl Lactylate; Calcium Stearate; Calcium Sulfate; Calrose; Camphor; Cane Sugar; Cane Vinegar; Canola (Rapeseed); Canola Oil (Rapeseed Oil); Caprylic Acid; Carageenan Chondrus Crispus; Carbonated Water; Carboxymethyl; Cellulose; Carmine; Camauba Wax; Carob Bean; Carob Bean Gum; Carob Flour; Carrageenan; Casein; Cassava Manihot Esculenta; Castor Oil; Catalase; Cellulose; Cellulose Ether; Cellulose Gum; Cetyl Alcohol; Cetyl Stearyl Alcohol; Champagne Vinegar; Channa (Chickpea); Chana Flour (chickpea flour); Cheeses; Chestnuts; Chickpea; Chlorella; Chocolate Liquor; Choline; Chloride; Chromium; Citrate; Chymosin; Citric Acid; Citrus Red No. 2; Cochineal; Cocoa; Cocoa Butter; Coconut; Coconut Vinegar; Collagen; Colloidal Silicon Dioxide; Confectioner's Glaze; Copernicia Cerifera; Copper Sulphate; Corn; Corn Gluten; Corn Masa Flour; corn meal; Corn Flour; corn starch; Corn Sugar; Corn Sugar Vinegar; Corn Syrup; Corn Syrup Solids; Corn Sweetener; Corn Vinegar; Corn Zein; Cortisone; Cotton Seed; Cotton Seed Oil; Cowitch; Cowpea; Cream of Tartar; Crospovidone; Curds; Cyanocobalamin; Cysteine, L; Dal (Lentils); D-Alpha-tocopherol; Dasheen Flour (Taro); Dates; D-Calcium Pantothenate; Delactosed Whey; Demineralized Whey; Desamidocollagen; Dextran; Dextrose; Diglycerides; Dioctyl Sodium; Dioctyl Sodium Solfosuccinate; Dipotassium Phosphate; Disodium Guanylate; Disodium Inosinate; Disodium Phosphate; Distilled Alcohols; Distilled Vinegar; Distilled White Vinegar; Dutch Processed Cocoa; EDTA (Ethylenediaminetetraacetic Acid); Eggs; Egg Yolk; Elastin; Ester Gum; Ethyl Alcohol; Ethylenediaminetetraacetic Acid; Ethyl Maltol; Ethyl Vanillin; Expeller Pressed Canola Oil; FD&C Blue No. 1 Dye; FD&C Blue No. 1 Lake; FD&C Blue No. 2 Dye; FD&C Blue No. 2 Lake; FD&C Green No. 3 Dye; FD&C Green No. 3 Lake; FD&C Red No. 3 Dye; FD&C Red No. 40 Dye; FD&C Red No. 40 Lake; FD&C Yellow No. 5 Dye; FD&C Yellow No. 6 Dye; FD&C Yellow No. 6 Lake; Ferric Orthophosphate; Ferrous Gluconate; Ferrous Fumerate; Ferrous Lactate; Ferrous Sulfate; Fish (fresh); Flaked Rice; Flax; Folacin; Folate; Folic Acid-Folacin; Formaldehyde; Fructose; Fruit (including dried); Fruit Vinegar; Fumaric Acid; Galactose; Garbanzo Beans; Gelatin; Glucoamylase; Gluconolactone; Glucose; Glucose Syrup; Glutamate (free); Glutamic Acid; Glutamine (amino acid); Glutinous Rice; Glutinous rice flour; Glycerides; Glycerin; Glycerol Monooleate; Glycol Monosterate; Glycol; Glycolic acid; Gram flour (chick peas); Grape Skin Extract; Grits, Corn; Guar Gum; Gum Acacia; Gum Arabic; Gum Base; Gum Tragacanth; Hemp; Hemp Seeds; Herbs; Herb Vinegar; Hexanedioic Acid; High Fructose Corn Syrup; Hominy; Honey; Hops; Horseradish (Pure); Hyacinth Bean; Hydrogen Peroxide; Hydrolyzed Caseinate; Hydrolyzed Meat Protein; Hydrolyzed Soy Protein; Hydroxypropyl Cellulose; Hydroxypropyl Methylcellulose; Hypromellose; Illepe; Iodine; Inulin; Invert Sugar; Iron Ammonium Citrate; Isinglass; Isolated Soy Protein; Isomalt; Jowar (Sorghum); Karaya Gum; Kasha (roasted buckwheat); Keratin; K-Carmine Color; K-Gelatin; Koshihikari (rice); Kudzu; Kudzu Root Starch; Lactalbumin Phosphate; Lactase; Lactic Acid; Lactitol; Lactose; Lactulose; Lanolin; Lard; L-cysteine; Lecithin; Lemon Grass; Lentils; Licorice; Licorice Extract; lipase; L-leucine; L-lysine; L-methionine; Locust Bean Gum; L-tryptophan; Magnesium Carbonate; Magnesium Hydroxide; Magnesium Oxide; Maize; Maize Waxy; Malic Acid; Maltitol; Maltodextrin; Maltol; Manganese Sulfate; Manioc; Masa; Masa Flour; Masa Harina; Meat (fresh); Medium Chain Triglycerides; Menhaden Oil; Methyl Cellulose; Microcrystalline Cellulose; Micro-particulated Egg White Protein; Milk; Milk Protein Isolate; Millet; Milo (Sorghum); Mineral Oil; Mineral Salts; Molybdenum Amino Acid Chelate; Monocalcium Phosphate; Monoglycerides; Mono and Diglycerides; Monopotassium Phosphate; Monosaccharides; Monosodium Glutamate (MSG); Monostearates; Mung Bean; Musk; Mustard Flour; Myristic Acid; Natural Smoke Flavor; Niacin-Niacinamide; Neotame; Niacin; Niacinamide; Nitrates; Nitrous Oxide; Non-fat Milk; Nuts (except wheat, rye & barley); Nut, Acron; Nut, Almond; Oils and Fats; Oleic Acid; Oleoresin; Olestra; Oleyl Alcohol/Oil; Orange B; Oryzanol; Palmitic Acid; Pantothenic Acid; Papain; Paprika; Paraffin; Patially Hydrogenated Cottonseed Oil; Patially Hydrogenated Soybean Oil; Peas; Pea—Chick; Pea—Cow; Pea Flour; Pea Starch; Peanuts; Peanut Flour; Pectin; Pectinase; Peppermint Oil; Peppers; Pepsin; Peru Balsam; Petrolatum; PGPR (Polyglycerol Polyricinoleate); Phenylalanine; Phosphoric Acid; Phosphoric Glycol; Pigeon; Peas; Polenta; Polydextrose; Polyethylene Glycol; Polyglycerol; Polyglycerol; Polyricinoleate (PGPR); Polysorbates; Polysorbate 60; Polysorbate 80; Potassium Benzoate; Potassium Caseinate; Potassium Citrate; Potassium Iodide; Potassium Lactate; Potassium Matabisulphite; Potassium Sorbate; Potatoes; Potato Flour; potato starch; Povidone; Prinus; Pristane; Propolis; Propylene Glycol; Monosterate; Propyl Gallate; protease; Psyllium; Pyridoxine Hydrochloride; Quinoa; Ragi; Raisin Vinegar; Rape; Recaldent; Reduced Iron; Rennet; Rennet Casein; Resinous Glaze; Reticulin; Riboflavin; Rice; Rice (Enriched); Rice Flour; Rice Starch; Rice Syrup; Rice Vinegar; Ricinoleic Acid; Romano Bean (chickpea); Rosematta; Rosin; Royal Jelly; Saccharin; Saffron; Sago; Sago Palm; Sago Flour; Sago Starch; Saifun (bean threads); Salt; Seaweed; Seeds (except wheat, rye & barley); Seed—Sesame; Seed—Sunflower; Shea; Sherry Vinegar; Silicon Dioxide; Soba (be sure its 100% buckwheat); Sodium Acid Pyrophosphate; Sodium Acetate; Sodium Alginate; Sodium Ascorbate; Sodium Benzoate; Sodium Caseinate; Sodium Citrate; Sodium Erythrobate; Sodium Hexametaphosphate; Sodium Lactate; Sodium Lauryl Sulfate; Sodium Metabisulphite; Sodium Nitrate; Sodium Phosphate; Sodium Polyphosphate; Sodium; Silaco Aluminate; Sodium; Stearoyl Lactylate; Sodium Sulphite; Sodium Stannate; Sodium Tripolyphosphate; Sorbic Acid; Sorbitan Monostearate; Sorbitol-Mannitol (can cause IBS symptoms); Sorghum; Sorghum Flour; Soy; Soybean; Soy Lecithin; Soy Protein; Soy Protein Isolate; Spices (pure); Spirits (Specific Types); Spirit Vinegar; Stearates; Stearamide; Stearamine; Stearic Acid; Stearyl Lactate; Stevia; Subflower Seed; Succotash (corn and beans); Sucralose; Sucrose; Sulfosuccinate; Sulfites; Sulfur Dioxide; Sweet Chestnut Flour; Tagatose; Tallow; Tapioca; tapioca flour; Tapioca Starch; Tara Gum; Taro; Tarro; Tarrow Root; Tartaric Acid; Tartrazine; TBHQ is Tetra or Tributylhydroquinone; Tea; Tea-Tree Oil; Teff; Teff Flour; Tepary Bean; Textured Vegetable Protein; Thiamin Hydrochloride; Thiamine Mononitrate; Thiamine Hydrochloride; Titanium Dioxide; Tofu (Soy Curd); Tolu Balsam; Torula Yeast; Tragacanth; Tragacanth Gum; Triacetin; Tricalcium Phosphate; Tri-Calcium Phosphate; Trypsin; Turmeric (Kurkuma); TVP; Tyrosine; Urad/Urid Beans; Urad/Urid Dal (peas) Vegetables; Urad/Urid flour; Urd; Vinegar (All except Malt); Vanilla Extract; Vanilla Flavoring; Vanillin; Vinegars (Specific Types); Vitamin A (retinol); Vitamin A Palmitate; Vitamin B1; Vitamin B-12; Vitamin B2; Vitamin B6; Vitamin D; Vitamin E Acetate; Waxy Maize; Whey; Whey Protein Concentrate; Whey Protein Isolate; White Vinegar; Wines; Wine Vinegars (& Balsamic); Wild Rice; xanthan Gum; Xylitol; Yam Flour; Yeast; Yogurt (plain, unflavored); Zinc Oxide; and Zinc Sulfate.

The following are the foods which are generally “forbidden” from the gluten free diet: Abyssinian Hard (Wheat Triticum durum); Alcohol (Spirits—Specific Types; Amp-Isostearoyl Hydrolyzed Wheat Protein; Atta Flour; Barley Grass (can contain seeds); Barley Hordeum vulgar; Barley Malt; Beer (most contain barley or wheat); Bleached Flour; Bran; bread Flour; Brewer's Yeast; Brown Flour; Bulgur (Bulgar Wheat/Nuts); Bulgur Wheat; Cereal; Binding; Chilton; Club Wheat (Triticum aestivum subspecies compactum) Common Wheat (Triticum aestivum); cookie Crumbs; Cookie Dough; Cookie Dough Pieces; Couscous; Criped Rice; Dinkle (Spelt); Disodium Wheatgermamido Peg-2 Sulfosuccinate; Durum wheat (Triticum durum); Edible Coatings; Edible Films; Edible Starch; Einkorn (Triticum monococcum); Emmer (Triticum dicoccon); Enriched Bleached Flour; Enriched Bleached Wheat Flour; Enriched Flour; Farina; Farina Graham; Farro; Filler; Flour; Fu (dried wheat gluten); Germ; Graham Flour; Granary Flour; Groats (barley, wheat); Hard Wheat; Heeng; Hing; Hordeum vulgare; Extract; Hydrolyzed Wheat Gluten; Hydrolyzed Wheat Protein; Hydrolyzed Wheat Protein Pg-Propyl Silanetriol; Hydrolyzed Wheat Starch; Hydroxypropyltrimonium; Hydrolyzed Wheat Protein; Kamut (pasta wheat); Kecap Manis (Soy Sauce); Ketjap Manis (Soy Sauce); Kluski Pasta; Maida (Indian wheat flour); Malt; Malted Barley Flour; Malted Milk; Malt Extract; Malt Syrup; Malt Flavoring; Malt Vinegar; Macha Wheat (Triticum aestivum); Matza; Matzah; Matzo; Matzo Semolina; Meringue; Meripro 711; Mir; Nishasta; Oriental Wheat (Triticum turanicum); Orzo Pasta; Pasta; Pearl Barley; Persian Wheat (Triticum carthlicum); Perungayam; Poulard Wheat (Triticum turgidum); Polish Wheat (Triticum polonicum); Rice Malt (if barley or Koji are used); Roux; Rusk; Rye; Seitan; Semolina; Semolina Triticum; Shot Wheat (Triticum aestivum); Small Spelt; Spirits (Specific Types); Spelt (Triticum spelta); Sprouted Wheat or Barley; Stearyldimoniumhydroxypropyl Hydrolyzed Wheat Protein; Strong Flour; Suet in Packets; Tabbouleh Tabouli; Teriyaki Sauce; Timopheevi Wheat (Triticum timopheevii); Triticale X triticosecale; Triticum Vulgare (Wheat); Flour Lipids; Triticum Vulgare (Wheat); Germ Extract; Triticum Vulgare (Wheat); Germ Oil; Udon (wheat noodles); Unbleached Flour; Vavilovi Wheat (Triticum aestivum); Vital Wheat Gluten; wheat; Abyssinian Hard Triticum durum; Wheat Amino acids; Wheat Bran Extract; Wheat, Bulgur; wheat Durum triticum; wheat Germ Extract; Wheat Germ Glycerides; Wheat Germ Oil; Wheat Germamidopropyldimonium Hydroxypropyl; Hydrolyzed Wheat Protein; Wheat Grass (can contain seeds); Wheat Nuts; Wheat Protein; Wheat Triticum aestivum; Wheat Triticum Monococcum; Wheat (Triticum Vulgare) Bran Extract; Whole-meal Flour; Wild Einkorn (Triticum boeotictim); and Wild Emmer (Triticum dicoccoides).

People with Celiac disease are more likely to have Autoimmune disorders such as rheumatoid arthritis, systemic lupus erythematosus, and Sjogren syndrome; Addison's disease; Down syndrome; Intestinal cancer; Intestinal lymphoma; Lactose intolerance; Thyroid disease; and/or Type 1 diabetes.

Treatment of a patient described herein who exhibits one or more symptoms of Celiac disease as well as one or more symptoms of the diseases or disorders described may be administered conventional therapy of such one or more symptoms of the diseases or disorders.

Patients typically experience the resolution of the findings of malnutrition, improved growth with resultant normal stature, and normalization of blood and biochemical laboratory studies. Such treatments may be combined with those of the compositions and treatments described herein to facilitate and/or advance resolution of one or more symptoms. Normal results from a follow-up endoscopy with biopsy several months after the diagnosis and treatment confirm the disease.

### EXAMPLES

### Example 1: Pancreatin

Pancreatin is a substance containing enzymes, principally amylase, lipase, and protease, obtained from the pancreas of the hog Sus scrofa Linne var. domesticus Gray (Fam. Suidae) or of the ox Bos Taurus Linne (Fam. Bocidae). Pancreatin contains, in each mg, not less than 25 U.S.P. units of amylase activity, not less than 2 U.S.P. units of lipase activity, and not less than 25 U.S.P. of protease activity. Pancreatin of a higher digestive power may be labeled as a whole-number multiple of the three minimum activities or may be diluted by admixture with lactose, or with sucrose containing not more than 3.25 percent of starch, or with pancreatin of a lower digestive power.

### Example 2: Reduction of Gastrointestinal Symptoms

A reduction in gastrointestinal symptoms associated with untreated or partially treated Celiac disease can be seen when the individuals are administered a digestive enzyme preparation high in proteases including chymotrypsin, trypsin, and other proteases. Said results are obtained within 3-10 days of administration. The methodology as described may be altered accordingly by one skilled in the art of administration of enzymes. One ordinarily skilled in the art would be able to administer the enzyme.

### Example 3

One example of an enzyme preparation comprised of amylases, proteases and lipases contains protease in an amount ranging between 155,000 and 310,000 U.S.P. units of protease activity, the main component of the enzyme preparation. The methodology as described may be altered accordingly as one skilled in the art of administration of enzymes or administration. One ordinarily skilled in the art would be able to administer the enzyme.

### Example 4

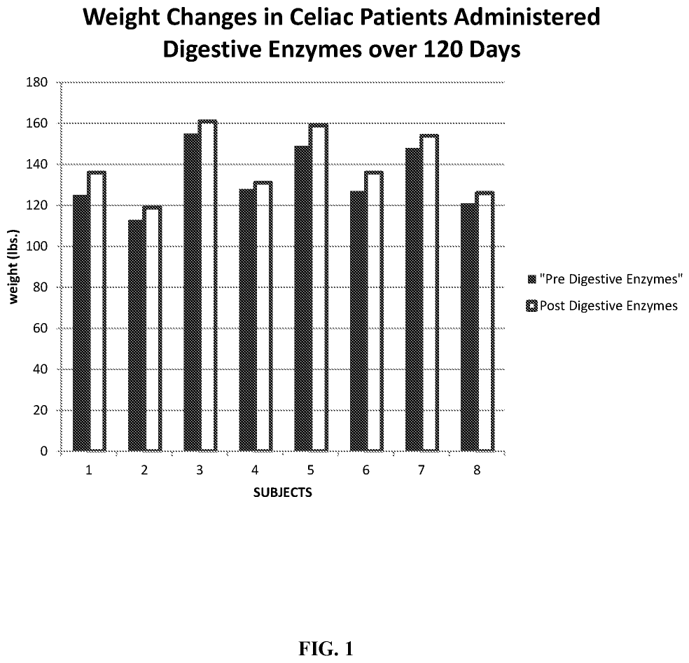

This example describes a method of treating Celiac disease which includes administering an enzyme preparation comprised of amylases, proteases and lipases. The protease strength range of between 155,000 and 310,000 units of protease activity is the main component of the enzyme preparation. The enzyme preparation administration given to those with Celiac disease resulted in an increase in weight gain (See Table 2 and FIG. 1).

TABLE 2

| | | | Celiac | weight | weight |
|---|---|---|---|---|---|
| | | | diagnosis | pre DE | post DE |
| Subject | Sex | AGE | Confirmed | 16 weeks (lbs) | 16 weeks (lbs) |
| 1 | M | 37 | Yes | 125 | 136 |
| 2 | F | 42 | Yes | 113 | 119 |
| 3 | F | 32 | Yes | 155 | 161 |
| 4 | F | 24 | Yes | 128 | 131 |
| 5 | M | 36 | Yes | 149 | 159 |
| 6 | F | 22 | Yes | 127 | 136 |
| 7 | M | 52 | Yes | 148 | 154 |
| 8 | F | 45 | Yes | 121 | 126 |

### Example 5

This example describes a method of treating Celiac disease which includes administering an enzyme preparation comprised of amylases, proteases and lipases. The protease strength of between 155,000 and 310,000 units of protease activity is the main component of the enzyme preparation. The enzyme administration given to those with Celiac disease resulted in a decrease in gastrointestinal pain, bloating, loose stools, constipation, diarrhea, flatulence and fatty “floating” stools.

A 34 year old male presented with a history of chronic diarrhea, floating and “fatty” type stools, who has difficulty maintaining weight due to Celiac disease. The patient was given enzyme therapy with high protease enzymes of at least 350,000 U.S.P. U/dose of protease three times daily with food. A continual gluten-free diet was maintained throughout the 120 days of treatment. There was no co-morbidity or other medication given. The individual suffered from diarrhea, with loose and frequent bowel movements every day regardless of the gluten-free diet. Post enzyme therapy, the individual demonstrated fewer bowel movements per day. On average there were 6-8 bowel movements per day which reduced to 2-3, and the stool was formed instead of soft and mushy. There was no odor associated with the bowel movements after 10 days of taking the enzyme preparation. On day 90 a stool test was performed whereby fecal fat content went from a +3 on day 1 to 0 on day 90. Further, the stool sunk to the bottom of the bowl whereas the stool previously floated, regardless of formation.

A 29 year old female presented with chronic constipation, flatulence and bloating. Causes other than Celiac disease were ruled out. She presented with continual difficulty after eating, and had been diagnosed with Celiac disease at age 15. She was administered 300,000 units of protease coupled with amylase and lipase three times daily with food. On day 12 her constipation reduced, and by day 30 she was able to stop her stool softener. On day 30, her bloating reduced with a disappearance of the flatulence. The individual continued to take her enzyme preparation with food. If the individual missed 3 consecutive doses while eating, the flatulence and bloating returned.

### Example 6

This example describes a method of treating Celiac disease which includes administering an enzyme preparation comprised of amylases, proteases and lipases. The protease strength of between 155,000 and 310,000 units of protease activity is the main component of the enzyme preparation. The enzyme administration given to those with Celiac disease resulted in a decrease in associated neurological symptoms including tingling, numbness, memory loss, tremor, irritability and seizure activity associated with celiac and related disorders according to the Tremor Scale described below.

TABLE 3
Tremor Scale (symptomatic complaint
of tremor in any part of the body)

| | |
|---|---|
| 0= | Absent (no tremor or writing impairment) |
| 1= | Slight and infrequently present (mild tremor, |
| | writing, and drawing of spiral |
| | minimally impaired) |
| 2= | Moderate; bothersome to most patients (writing |
| | and drawing of spiral moderately |
| | impaired) |
| 3= | Severe tremor (writing and drawing severely |
| | impaired; interferes with many |
| | activities such as drinking liquids) |
| 4= | Marked tremor (interferes with most activities) |

### Example 7

This example describes a method of treating Celiac disease which includes administering an enzyme preparation comprised of amylases, proteases and lipases. The protease strength of between 155,000 and 310,000 units of protease activity is the main component of the enzyme preparation. The enzyme administration given to those with Celiac disease resulted in a reduction in symptoms which are non GI or neurological: nose bleeds, osteoporosis and muscle cramps associated with celiac and related disorders.

While preferred embodiments have been shown and described herein, it will be obvious to those skilled in the art that such embodiments are provided by way of example only. Numerous variations, changes, and substitutions will now occur to those skilled in the art without departing from the embodiments. It should be understood that various alternatives to the embodiments described herein may be employed in practicing the described compositions and methods. It is intended that the following claims define the scope of the embodiments and that methods and structures within the scope of these claims and their equivalents be covered thereby.

What is claimed is:

1. A method for preventing or treating Celiac disease or a related disorder in an individual, comprising administering a therapeutically effective amount of a composition that comprises digestive enzymes to the individual, wherein the one or more symptoms of Celiac disease or a related disorder are treated.

2. The method of claim 1 wherein the composition is administered until one or more symptoms of Celiac disease or a related disorder are partially or completely resolved

3. The method of claim 2, wherein the one or more symptoms are selected from the group consisting of abdominal pain, bloating, gas, or indigestion, constipation, decreased appetite, diarrhea, lactose intolerance, nausea and vomiting, unexplained weight loss, bruising easily, depression, anxiety, chronic headaches, migraines, fatigue, growth delay in children, hair loss, itchy skin, missed menstrual periods, mouth ulcers, muscle cramps and joint pain, nosebleeds, seizures, tingling or numbness in the hands or feet, unexplained short height, defects in the tooth enamel and changes in tooth color, delayed puberty, irritable and fussy behavior, poor weight gain, slowed growth and shorter than normal height, or a combination thereof.

4. The method of claim 1, wherein the one or more symptoms are reduced in severity or duration by about 2%, about 5%, about 10%, about 15%, about 20%, about 25%, about 30%, about 35%, about 40%, about 45%, about 50%, about 55%, about 60%, about 65%, about 70%, about 75%, about 80%, about 90%, about 95%, or about 100%.

5. The method of claim 2, wherein the composition is administered 1, 2, 3 or 4 times a day until one or more symptoms are partially or completely resolved.

6. The method of claim 2, wherein the composition is administered 1, 2, 3 or 4 times a week until one or more symptoms are partially or completely resolved.

7. The method of claim 1, wherein the method further comprises administering one or more additional agents or treatment regimens.

8. The method of claim 7, wherein the one or more additional agents or treatment regimens comprises IgA or a gluten-free diet.

9. The method of claim 1, wherein the composition comprising digestive enzymes comprises amylases, lipases, proteases, or a combination thereof.

10. The method of claim 9, wherein the composition further comprises chymotrypsin, trypsin, pancreatin, papain, or a combination thereof.

11. The method of claim 9, wherein the digestive enzymes are derived from a source selected from the group consisting of animal enzymes, plant enzymes, synthetic enzymes, and a combination thereof.

12. The method of claim 11, wherein the animal enzyme is derived from a mammal.

13. The method of claim 12, wherein the mammal is a pig.

14. The method of claim 11, wherein the animal enzyme is derived from a pancreas.

15. The method of claim 1, wherein the compositions comprising digestive enzymes are manufactured using a technology selected from the group consisting of enteric coating, lipid encapsulation, direct compression, dry granulation, wet granulation, and a combination thereof.

16. The method of claim 1, wherein the composition is administered orally.

17. The method of claim 1, wherein the composition is a dosage formulation selected from the group consisting of pills, tablets, capsules, microcapsules, mini-capsules, time released capsules, mini-tabs, sprinkles, suppository and a combination thereof.

18. The method of claim 9, wherein the total amount of protease in the composition is from about 10,000 to about 1,500,000; from about 25,000 to about 1,000,000; from about 50,000 to about 750,000; from about 75,000 to about 500,000; from about 85,000 to about 250,000; from about 95,000 to about 200,000; or from about 110,000 to about 150,000 U.S.P. units/dose.

19. The method of claim 18, wherein the total amount of protease in the composition is about 5,000; about 7,500; about 10,000; about 15,000; about 20,000; about 25,000; about 30,000; about 35,000; about 40,000; about 50,000; about 55,000; about 65,000; about 70,000; about 75,000; about 90,000; about 95,000; about 100,000; about 110,000; about 115,000; about 130,000; about 140,000; about 140,400; about 150,000; about 155,000; about 160,000; about 170,000; about 175,000; about 180,000; about 190,000; about 195,000; about 200,000; about 210,000; about 220,000; about 230,000; about 240,000; about 250,000; about 280,000; about 290,000; about 300,000; about 310,000; about 320,000; about 330,000; about 340,000; about 350,000; about 360,000; about 370,000; about 380,000; about 390,000; about 400,000; about 410,000; about 420,000; about 430,000; about 440,000; about 450,000; about 465,000; about 470,000; about 480,000; about 490,000; about 500,000; about 510,000; about 520,000; about 530,000; about 540,000; about 550,000; about 560,000; about 570,000; about 580,000; about 590,000; about 600,000; about 610,000; about 620,000; about 630,000; about 640,000; about 650,000; about 660,000; about 670,000; about 680,000; about 690,000; about 700,000; about 710,000; about 720,000; about 730,000; about 740,000; about 750,000; about 760,000; about 770,000; about 780,000; about 790,000; about 800,000; about 810,000; about 820,000; about 830,000; about 840,000; about 850,000; about 860,000; about 870,000; about 880,000; about 890,000; about 900,000; about 910,000; about 920,000; about 930,000; about 940,000; about 950,000; about 960,000; about 970,000; about 980,000; about 990,000; about 1,000,000; about 1,010,000; about 1,020,000; about 1,020,000; about 1,030,000; about 1,040,000; about 1,050,000; about 1,060,000; about 1,070,000; about 1,080,000; about 1,090,000; about 1,100,000; about 1,100,000; about 1,120,000; about 1,130,000; about 1,140,000; about 1,150,000; about 1,170,000; about 1,190,000; about 1,200,000; about 1,210,000; about 1,250,000; about 1,300,000; about 1,350,000; about 1,400,000; about 1,450,000; or about 1,500,000; about 1,200,000; about 1,250,000; about 1,300,000; about 1,350,000; about 1,400,000; about 1,450,000; or about 1,500,000 U.S.P. units/dose.

20. The method of claim 9, wherein the total amount of amylase in the composition is from about 1,000 to about 15,000,000; from about 5,000 to about 1,000,000; from about 15,000 to about 750,000; from about 50,000 to about 500,000; from about 75,000 to about 250,000; from about 95,000 to about 200,000; or from about 100,000 to about 150,000 U.S.P. units/dose.

21. The method of claim 20, wherein the total amount of amylase in the composition is about 1,000; about 3,000; about 5,000; about 7,500; about 10,000; about 15,000; about 20,000; about 25,000; about 30,000; about 35,000; about 40,000; about 50,000; about 65,000; about 70,000; about 75,000; about 100,000; about 140,000; about 144,000; about 210,000; about 280,000; about 350,000; about 420,000; about 490,000; about 500,000; about 560,000; about 630,000; about 700,000; about 770,000; about 840,000; about 910,000; about 980,000; about 1,000,000; about 1,050,000; about 2,000,000; about 3,000,000; about 4,000,000; about 5,000,000; about 6,000,000; about 7,000,000; about 8,000,000; about 9,000,000; about 10,000,000; about 11,000,000; about 12,000,000; about 13,000,000; about 14,000,000; and about 15,000,000 U.S.P. units/dose.

22. The method of claim 9, wherein the total amount of lipase in the composition is from about 1,500 to about 282,000; from about 5,000 to about 200,000; from about 5,000 to about 150,000; from about 75,000 to about 100,000; from about 10,000 to about 75,000; from about 15,000 to about 50,000; or from about 20,000 to about 40,000 U.S.P. units/dose.

23. The method of claim 22, wherein the total amount of lipase is about 1,500; about 1,880; about 2,000; about 3,000; about 5,000; about 7,500; about 8,400; about 10,000; about 15,000; about 16,800; about 20,000; about 23,000; about 23,040; about 25,000; about 30,000; about 33,600; about 40,000; about 50,000; about 50,400; about 65,000; about 67,200; about 75,000; about 84,000; about 100,000; about 100,800; about 117,600; about 125,000; about 134,400; about 150,000; about 151,200; about 168,000; about 184,800; about 200,000; about 201,600; about 218,400; about 235,200; about 250,000; about 252,000; and about 282,000 U.S.P. units/dose.

24. The method of claim 1, wherein the pharmaceutical composition comprises about 23,000 U.S.P. units/dose of lipase, about 144,000 U.S.P. units/dose of amylase and about 140,000 U.S.P. units/dose of protease.

25. The method of claim 1, wherein the pharmaceutical composition comprises about 23,040 U.S.P. units/dose of lipase, about 144,000 U.S.P. units/dose of amylase and about 140,400 U.S.P. units/dose of protease.

26. The method of claim 1, wherein the pharmaceutical composition comprises about 16,800 U.S.P. units/dose of lipase, about 70,000 U.S.P. units/dose of protease, and about 70,000 U.S.P. units/dose of amylase.

27. The method of claim 1, wherein the pharmaceutical composition comprises about 16,800 U.S.P. units/dose of lipase, about 110,000 U.S.P. units/dose of protease, and about 70,000 U.S.P. units/dose of amylase.

28. The method of claim 9, wherein the pharmaceutical composition comprises about 8,400 U.S.P. units lipase, 35,000 U.S.P. units protease, and about 35,000 U.S.P. units amylase.

29. The method of claim 9, wherein the pharmaceutical composition comprises about 16,800 U.S.P. units lipase, about 70,000 U.S.P. units protease, and about 70,000 U.S.P. units amylase.

30. The method of claim 9, wherein the pharmaceutical composition comprises about 33,600 U.S.P. units lipase, about 140,000 U.S.P. units protease, and about 140,000 U.S.P. units amylase.

31. The method of claim 9, wherein the pharmaceutical composition comprises about 50,400 U.S.P. units lipase, about 210,000 U.S.P. units protease, and about 210,000 U.S.P. units amylase.

32. The method of claim 9, wherein the pharmaceutical composition comprises about 67,200 U.S.P. units lipase, about 280,000 U.S.P. units protease, and about 280,000 U.S.P. units amylase.

33. The method of claim 9, wherein the pharmaceutical composition comprises about 84,000 U.S.P. units lipase, about 350,000 U.S.P. units protease, and about 350,000 U.S.P. units amylase.

34. The method of claim 9, wherein the pharmaceutical composition comprises about 100,800 U.S.P. units lipase, about 420,000 U.S.P. units protease, and about 420,000 U.S.P. units amylase.

35. The method of claim 9, wherein the pharmaceutical composition comprises about 117,600 U.S.P. units lipase, about 490,000 U.S.P. units protease, and about 490,000 U.S.P. units amylase.

36. The method of claim 9, wherein the pharmaceutical composition comprises about 134,400 U.S.P. units lipase, about 560,000 U.S.P. units protease, and about 560,000 U.S.P. units amylase.

37. The method of claim 9, wherein the pharmaceutical composition comprises about 151,200 U.S.P. units lipase, about 630,000 U.S.P. units protease, and about 630,000 U.S.P. units amylase.

38. The method of claim 9, wherein the pharmaceutical composition comprises about 168,000 U.S.P. units lipase, about 700,000 U.S.P. units protease, and about 700,000 U.S.P. units amylase.

39. The method of claim 9, wherein the pharmaceutical composition comprises about 184,800 U.S.P. units lipase, about 770,000 U.S.P. units protease, and about 770,000 U.S.P. units amylase.

40. The method of claim 9, wherein the pharmaceutical composition comprises about 201,600 U.S.P. units lipase, about 840,000 U.S.P. units protease, and about 840,000 U.S.P. units amylase.

41. The method of claim 9, wherein the pharmaceutical composition comprises about 218,400 U.S.P. units lipase, about 910,000 U.S.P. units protease, and about 910,000 U.S.P. units amylase.

42. The method of claim 9, wherein the pharmaceutical composition comprises about 235,200 U.S.P. units lipase, about 980,000 U.S.P. units protease, and about 980,000 U.S.P. units amylase.

43. The method of claim 9, wherein the pharmaceutical composition comprises about 252,000 U.S.P. units lipase, about 1,050,000 U.S.P. units protease, and about 1,050,000 U.S.P. units amylase.

44. The method of any one of claims 28-43, further comprising a supplemental amount of protease selected from the group consisting of about 0 U.S.P. units; about 20,000 U.S.P. units; about 40,000 U.S.P. units; about 60,000 U.S.P. units; about 80,000 U.S.P. units; about 100,000 U.S.P. units; about 120,000 U.S.P. units; about 140,000 U.S.P. units; and about 160,000 U.S.P. units protease.

45. The method of claim 1, wherein the treatment increases weight gain of the individual.

46. A pharmaceutical composition comprising a therapeutically effective amount of digestive enzymes for use in the prevention or treatment of Celiac disease.

47. The pharmaceutical composition of claim 46, wherein administration of the composition to a subject partially or fully resolves one or more symptoms of Celiac disease.

48. The pharmaceutical composition of claim 47, wherein the one or more symptoms are selected from the group consisting of abdominal pain, bloating, gas, or indigestion, constipation, decreased appetite, diarrhea, lactose intolerance, nausea and vomiting, unexplained weight loss, bruising easily, depression, anxiety, chronic headaches, migraines, fatigue, growth delay in children, hair loss, itchy skin, missed menstrual periods, mouth ulcers, muscle cramps and joint pain, nosebleeds, seizures, tingling or numbness in the hands or feet, unexplained short height, defects in the tooth enamel and changes in tooth color, delayed puberty, irritable and fussy behavior, poor weight gain, slowed growth and shorter than normal height, or a combination thereof.

49. The pharmaceutical composition of claim 46, wherein the composition comprising digestive enzymes comprises amylases, lipases, proteases, or a combination thereof.

50. The pharmaceutical composition of claim 49, wherein the composition further comprises chymotrypsin, trypsin, pancreatin, papain, or a combination thereof.

51. The pharmaceutical composition of claim 49, wherein the digestive enzymes are derived from a source selected from the group consisting of animal enzymes, plant enzymes, synthetic enzymes, and a combination thereof.

52. The pharmaceutical composition of claim 51, wherein the animal enzyme is derived from a mammal.

53. The pharmaceutical composition of claim 52, wherein the mammal is a pig.

54. The pharmaceutical composition of claim 51, wherein the animal enzyme is derived from a pancreas.

55. The pharmaceutical composition of claim 46, wherein the compositions comprising digestive enzymes are manufactured using a technology selected from the group consisting of enteric coating, lipid encapsulation, direct compression, dry granulation, wet granulation, and a combination thereof.

56. The pharmaceutical composition of claim 46, wherein the composition is formulated for oral administration.

57. The pharmaceutical composition of claim 46, wherein the composition is a dosage formulation selected from the group consisting of pills, tablets, capsules, microcapsules, mini-capsules, time released capsules, mini-tabs, sprinkles, suppository and a combination thereof.

58. The pharmaceutical composition of claim 49, wherein the total amount of protease in the composition is from about 10,000 to about 1,500,000; from about 25,000 to about 1,000,000; from about 50,000 to about 750,000; from about 75,000 to about 500,000; from about 85,000 to about 250,000; from about 95,000 to about 200,000; or from about 110,000 to about 150,000 U.S.P. units/dose.

59. The pharmaceutical composition of claim 58, wherein the total amount of protease in the composition is about 5,000; about 7,500; about 10,000; about 15,000; about 20,000; about 25,000; about 30,000; about 35,000; about 40,000; about 50,000; about 55,000; about 65,000; about 70,000; about 75,000; about 90,000; about 95,000; about 100,000; about 110,000; about 115,000; about 130,000; about 140,000; about 140,400; about 150,000; about 155,000; about 160,000; about 170,000; about 175,000; about 180,000; about 190,000; about 195,000; about 200,000; about 210,000; about 220,000; about 230,000; about 240,000; about 250,000; about 280,000; about 290,000; about 300,000; about 310,000; about 320,000; about 330,000; about 340,000; about 350,000; about 360,000; about 370,000; about 380,000; about 390,000; about 400,000; about 410,000; about 420,000; about 430,000; about 440,000; about 450,000; about 465,000; about 470,000; about 480,000; about 490,000; about 500,000; about 510,000; about 520,000; about 530,000; about 540,000; about 550,000; about 560,000; about 570,000; about 580,000; about 590,000; about 600,000; about 610,000; about 620,000; about 630,000; about 640,000; about 650,000; about 660,000; about 670,000; about 680,000; about 690,000; about 700,000; about 710,000; about 720,000; about 730,000; about 740,000; about 750,000; about 760,000; about 770,000; about 780,000; about 790,000; about 800,000; about 810,000; about 820,000; about 830,000; about 840,000; about 850,000; about 860,000; about 870,000; about 880,000; about 890,000; about 900,000; about 910,000; about 920,000; about 930,000; about 940,000; about 950,000; about 960,000; about 970,000; about 980,000; about 990,000; about 1,000,000; about 1,010,000; about 1,020,000; about 1,020,000; about 1,030,000; about 1,040,000; about 1,050,000; about 1,060,000; about 1,070,000; about 1,080,000; about 1,090,000; about 1,100,000; about 1,100,000; about 1,120,000; about 1,130,000; about 1,140,000; about 1,150,000; about 1,170,000; about 1,190,000; about 1,200,000; about 1,210,000; about 1,250,000; about 1,300,000; about 1,350,000; about 1,400,000; about 1,450,000; or about 1,500,000; about 1,200,000; about 1,250,000; about 1,300,000; about 1,350,000; about 1,400,000; about 1,450,000; or about 1,500,000 U.S.P. units/dose.

60. The pharmaceutical composition of claim 49, wherein the total amount of amylase in the composition is from about 1,000 to about 15,000,000; from about 5,000 to about 1,000,000; from about 15,000 to about 750,000; from about 50,000 to about 500,000; from about 75,000 to about 250,000; from about 95,000 to about 200,000; or from about 100,000 to about 150,000 U.S.P. units/dose.

61. The pharmaceutical composition of claim 60, wherein the total amount of amylase in the composition is about 1,000; about 3,000; about 5,000; about 7,500; about 10,000; about 15,000; about 20,000; about 25,000; about 30,000; about 35,000; about 40,000; about 50,000; about 65,000; about 70,000; about 75,000; about 100,000; about 140,000; about 144,000; about 210,000; about 280,000; about 350,000; about 420,000; about 490,000; about 500,000; about 560,000; about 630,000; about 700,000; about 770,000; about 840,000; about 910,000; about 980,000; about 1,000,000; about 1,050,000; about 2,000,000; about 3,000,000; about 4,000,000; about 5,000,000; about 6,000,000; about 7,000,000; about 8,000,000; about 9,000,000; about 10,000,000; about 11,000,000; about 12,000,000; about 13,000,000; about 14,000,000; and about 15,000,000 U.S.P. units/dose.

62. The pharmaceutical composition of claim 49, wherein the total amount of lipase in the composition is from about 1,500 to about 282,000; from about 5,000 to about 200,000; from about 5,000 to about 150,000; from about 75,000 to about 100,000; from about 10,000 to about 75,000; from about 15,000 to about 50,000; or from about 20,000 to about 40,000 U.S.P. units/dose.

63. The pharmaceutical composition of claim 62, wherein the total amount of lipase in the composition is about 1,500; about 1,880; about 2,000; about 3,000; about 5,000; about 7,500; about 8,400; about 10,000; about 15,000; about 16,800; about 20,000; about 23,000; about 23,040; about 25,000; about 30,000; about 33,600; about 40,000; about 50,000; about 50,400; about 65,000; about 67,200; about 75,000; about 84,000; about 100,000; about 100,800; about 117,600; about 125,000; about 134,400; about 150,000; about 151,200; about 168,000; about 184,800; about 200,000; about 201,600; about 218,400; about 235,200; about 250,000; about 252,000; and about 282,000 U.S.P. units/dose.

64. A pharmaceutical composition comprising digestive enzymes, wherein the pharmaceutical composition comprises about 23,000 U.S.P. units/dose of lipase, about 144,000 U.S.P. units/dose of amylase and about 140,000 U.S.P. units/dose of protease.

65. A pharmaceutical composition comprising digestive enzymes, wherein the pharmaceutical composition comprises about 23,040 U.S.P. units/dose of lipase, about 144,000 U.S.P. units/dose of amylase and about 140,400 U.S.P. units/dose of protease.

66. A pharmaceutical composition comprising digestive enzymes, wherein the digestive enzymes comprise about 16,800 U.S.P. units/dose of lipase, about 70,000 U.S.P. units/dose of protease, and about 70,000 U.S.P. units/dose of amylase.

67. A pharmaceutical composition comprising digestive enzymes, wherein the digestive enzymes comprise about 16,800 U.S.P. units/dose of lipase, about 110,000 U.S.P. units/dose of protease, and about 70,000 U.S.P. units/dose of amylase.

68. A dose of a pharmaceutical composition comprising digestive enzymes, wherein the dose comprises about 8,400 U.S.P. units lipase, 35,000 U.S.P. units protease, and about 35,000 U.S.P. units amylase.

69. A dose of a pharmaceutical composition comprising digestive enzymes, wherein the dose comprises about 16,800 U.S.P. units lipase, about 70,000 U.S.P. units protease, and about 70,000 U.S.P. units amylase.

70. A dose of a pharmaceutical composition comprising digestive enzymes, wherein the dose comprises about 33,600 U.S.P. units lipase, about 140,000 U.S.P. units protease, and about 140,000 U.S.P. units amylase.

71. A dose of a pharmaceutical composition comprising digestive enzymes, wherein the dose comprises about 50,400 U.S.P. units lipase, about 210,000 U.S.P. units protease, and about 210,000 U.S.P. units amylase.

72. A dose of a pharmaceutical composition comprising digestive enzymes, wherein the dose comprises about 67,200 U.S.P. units lipase, about 280,000 U.S.P. units protease, and about 280,000 U.S.P. units amylase.

73. A dose of a pharmaceutical composition comprising digestive enzymes, wherein the dose comprises about 84,000 U.S.P. units lipase, about 350,000 U.S.P. units protease, and about 350,000 U.S.P. units amylase.

74. A dose of a pharmaceutical composition comprising digestive enzymes, wherein the dose comprises about 100,800 U.S.P. units lipase, about 420,000 U.S.P. units protease, and about 420,000 U.S.P. units amylase.

75. A dose of a pharmaceutical composition comprising digestive enzymes, wherein the dose comprises about 117,600 U.S.P. units lipase, about 490,000 U.S.P. units protease, and about 490,000 U.S.P. units amylase.

76. A dose of a pharmaceutical composition comprising digestive enzymes, wherein the dose comprises about 134,400 U.S.P. units lipase, about 560,000 U.S.P. units protease, and about 560,000 U.S.P. units amylase.

77. A dose of a pharmaceutical composition comprising digestive enzymes, wherein the dose comprises about 151,200 U.S.P. units lipase, about 630,000 U.S.P. units protease, and about 630,000 U.S.P. units amylase.

78. A dose of a pharmaceutical composition comprising digestive enzymes, wherein the dose comprises about 168,000 U.S.P. units lipase, about 700,000 U.S.P. units protease, and about 700,000 U.S.P. units amylase.

79. A dose of a pharmaceutical composition comprising digestive enzymes, wherein the dose comprises about 184,800 U.S.P. units lipase, about 770,000 U.S.P. units protease, and about 770,000 U.S.P. units amylase.

80. A dose of a pharmaceutical composition comprising digestive enzymes, wherein the dose comprises about 201,600 U.S.P. units lipase, about 840,000 U.S.P. units protease, and about 840,000 U.S.P. units amylase.

81. A dose of a pharmaceutical composition comprising digestive enzymes, wherein the dose comprises about 218,400 U.S.P. units lipase, about 910,000 U.S.P. units protease, and about 910,000 U.S.P. units amylase.

82. A dose of a pharmaceutical composition comprising digestive enzymes, wherein the dose comprises about 235,200 U.S.P. units lipase, about 980,000 U.S.P. units protease, and about 980,000 U.S.P. units amylase.

83. A dose of a pharmaceutical composition comprising digestive enzymes, wherein the dose comprises about 252,000 U.S.P. units lipase, about 1,050,000 U.S.P. units protease, and about 1,050,000 U.S.P. units amylase.

84. A dose of any one of claims 64-83, further comprising a supplemental amount of protease selected from the group consisting of about 0 U.S.P. units; about 20,000 U.S.P. units; about 40,000 U.S.P. units; about 60,000 U.S.P. units; about 80,000 U.S.P. units; about 100,000 U.S.P. units; about 120,000 U.S.P. units; about 140,000 U.S.P. units; and about 160,000 U.S.P. units protease.

85. A method of inducing weight gain in a subject, comprising administering to the subject a pharmaceutical composition comprising a therapeutically effective amount of digestive enzymes to the subject.

86. The method of claim 85, wherein the composition is administered 1, 2, 3 or 4 times a day until the subject begins gaining weight.

87. The method of claim 85, wherein the composition is administered 1, 2, 3 or 4 times a week until the subject begins gaining weight.

88. The method of claim 85, wherein the method further comprises modifying the diet of the subject.

89. The method of claim 88, wherein the diet is a gluten-free diet.

90. The method of claim 85, wherein the composition comprising digestive enzymes comprises amylases, lipases, proteases, or a combination thereof.

91. The method of claim 90, wherein the composition further comprises chymotrypsin, trypsin, pancreatin, papain, or a combination thereof.

92. The method of claim 85, wherein the digestive enzymes are derived from a source selected from the group consisting of animal enzymes, plant enzymes, synthetic enzymes, and a combination thereof.

93. The method of claim 92, wherein the animal enzyme is derived from a mammal.

94. The method of claim 93, wherein the mammal is a pig.

95. The method of claim 92, wherein the animal enzyme is derived from a pancreas.

96. The method of claim 85, wherein the compositions comprising digestive enzymes are manufactured using a technology selected from the group consisting of enteric coating, lipid encapsulation, direct compression, dry granulation, wet granulation, and a combination thereof.

97. The method of claim 85, wherein the composition is administered orally.

98. The method of claim 85, wherein the composition is a dosage formulation selected from the group consisting of pills, tablets, capsules, microcapsules, mini-capsules, time released capsules, mini-tabs, sprinkles, suppository and a combination thereof.

99. The method of claim 90, wherein the total amount of protease in the composition is from about 10,000 to about 1,500,000; from about 25,000 to about 1,000,000; from about 50,000 to about 750,000; from about 75,000 to about 500,000; from about 85,000 to about 250,000; from about 95,000 to about 200,000; or from about 110,000 to about 150,000 U.S.P. units/dose.

100. The method of claim 99, wherein the total amount of protease in the composition is about 5,000; about 7,500; about 10,000; about 15,000; about 20,000; about 25,000; about 30,000; about 35,000; about 40,000; about 50,000; about 55,000; about 65,000; about 70,000; about 75,000; about 90,000; about 95,000; about 100,000; about 110,000; about 115,000; about 130,000; about 140,000; about 140,400; about 150,000; about 155,000; about 160,000; about 170,000; about 175,000; about 180,000; about 190,000; about 195,000; about 200,000; about 210,000; about 220,000; about 230,000; about 240,000; about 250,000; about 280,000; about 290,000; about 300,000; about 310,000; about 320,000; about 330,000; about 340,000; about 350,000; about 360,000; about 370,000; about 380,000; about 390,000; about 400,000; about 410,000; about 420,000; about 430,000; about 440,000; about 450,000; about 465,000; about 470,000; about 480,000; about 490,000; about 500,000; about 510,000; about 520,000; about 530,000; about 540,000; about 550,000; about 560,000; about 570,000; about 580,000; about 590,000; about 600,000; about 610,000; about 620,000; about 630,000; about 640,000; about 650,000; about 660,000; about 670,000; about 680,000; about 690,000; about 700,000; about 710,000; about 720,000; about 730,000; about 740,000; about 750,000; about 760,000; about 770,000; about 780,000; about 790,000; about 800,000; about 810,000; about 820,000; about 830,000; about 840,000; about 850,000; about 860,000; about 870,000; about 880,000; about 890,000; about 900,000; about 910,000; about 920,000; about 930,000; about 940,000; about 950,000; about 960,000; about 970,000; about 980,000; about 990,000; about 1,000,000; about 1,010,000; about 1,020,000; about 1,020,000; about 1,030,000; about 1,040,000; about 1,050,000; about 1,060,000; about 1,070,000; about 1,080,000; about 1,090,000; about 1,100,000; about 1,100,000; about 1,120,000; about 1,130,000; about 1,140,000; about 1,150,000; about 1,170,000; about 1,190,000; about 1,200,000; about 1,210,000; about 1,250,000; about 1,300,000; about 1,350,000; about 1,400,000; about 1,450,000; or about 1,500,000; about 1,200,000; about 1,250,000; about 1,300,000; about 1,350,000; about 1,400,000; about 1,450,000; or about 1,500,000 U.S.P. units/dose.

101. The method of claim 90, wherein the total amount of amylase in the composition is from about 1,000 to about 15,000,000; from about 5,000 to about 1,000,000; from about 15,000 to about 750,000; from about 50,000 to about 500,000; from about 75,000 to about 250,000; from about 95,000 to about 200,000; or from about 100,000 to about 150,000 U.S.P. units/dose.

102. The method of claim 101, wherein the total amount of amylase in the composition is about 1,000; about 3,000; about 5,000; about 7,500; about 10,000; about 15,000; about 20,000; about 25,000; about 30,000; about 35,000; about 40,000; about 50,000; about 65,000; about 70,000; about 75,000; about 100,000; about 140,000; about 144,000; about 210,000; about 280,000; about 350,000; about 420,000; about 490,000; about 500,000; about 560,000; about 630,000; about 700,000; about 770,000; about 840,000; about 910,000; about 980,000; about 1,000,000; about 1,050,000; about 2,000,000; about 3,000,000; about 4,000,000; about 5,000,000; about 6,000,000; about 7,000,000; about 8,000,000; about 9,000,000; about 10,000,000; about 11,000,000; about 12,000,000; about 13,000,000; about 14,000,000; and about 15,000,000 U.S.P. units/dose.

103. The method of claim 90, wherein the total amount of lipase in the composition is from about 1,500 to about 282,000; from about 5,000 to about 200,000; from about 5,000 to about 150,000; from about 75,000 to about 100,000; from about 10,000 to about 75,000; from about 15,000 to about 50,000; or from about 20,000 to about 40,000 U.S.P. units/dose.

104. The method of claim 103, wherein the total amount of lipase in the composition is about 1,500; about 1,880; about 2,000; about 3,000; about 5,000; about 7,500; about 8,400; about 10,000; about 15,000; about 16,800; about 20,000; about 23,000; about 23,040; about 25,000; about 30,000; about 33,600; about 40,000; about 50,000; about 50,400; about 65,000; about 67,200; about 75,000; about 84,000; about 100,000; about 100,800; about 117,600; about 125,000; about 134,400; about 150,000; about 151,200; about 168,000; about 184,800; about 200,000; about 201,600; about 218,400; about 235,200; about 250,000; about 252,000; and about 282,000 U.S.P. units/dose.

105. The method of claim 85, wherein the subject has Celiac disease.

106. The method of claim 85, wherein the pharmaceutical composition comprises about 23,000 U.S.P. units/dose of lipase, about 144,000 U.S.P. units/dose of amylase and about 140,000 U.S.P. units/dose of protease.

107. The method of claim 85, wherein the pharmaceutical composition comprises about 23,040 U.S.P. units/dose of lipase, about 144,000 U.S.P. units/dose of amylase and about 140,400 U.S.P. units/dose of protease.

108. The method of claim 85, wherein the pharmaceutical composition comprises digestive enzymes, wherein the digestive enzymes comprise about 16,800 U.S.P. units/dose of lipase, about 70,000 U.S.P. units/dose of protease, and about 70,000 U.S.P. units/dose of amylase.

109. The method of claim 85, wherein the pharmaceutical composition comprises digestive enzymes, wherein the digestive enzymes comprise about 16,800 U.S.P. units/dose of lipase, about 110,000 U.S.P. units/dose of protease, and about 70,000 U.S.P. units/dose of amylase.

110. The method of claim 90, wherein the pharmaceutical composition comprises about 8,400 U.S.P. units lipase, 35,000 U.S.P. units protease, and about 35,000 U.S.P. units amylase.

111. The method of claim 90, wherein the pharmaceutical composition comprises about 16,800 U.S.P. units lipase, about 70,000 U.S.P. units protease, and about 70,000 U.S.P. units amylase.

112. The method of claim 90, wherein the pharmaceutical composition comprises about 33,600 U.S.P. units lipase, about 140,000 U.S.P. units protease, and about 140,000 U.S.P. units amylase.

113. The method of claim 90, wherein the pharmaceutical composition comprises about 50,400 U.S.P. units lipase, about 210,000 U.S.P. units protease, and about 210,000 U.S.P. units amylase.

114. The method of claim 90, wherein the pharmaceutical composition comprises about 67,200 U.S.P. units lipase, about 280,000 U.S.P. units protease, and about 280,000 U.S.P. units amylase.

115. The method of claim 90, wherein the pharmaceutical composition comprises about 84,000 U.S.P. units lipase, about 350,000 U.S.P. units protease, and about 350,000 U.S.P. units amylase.

116. The method of claim 90, wherein the pharmaceutical composition comprises about 100,800 U.S.P. units lipase, about 420,000 U.S.P. units protease, and about 420,000 U.S.P. units amylase.

117. The method of claim 90, wherein the pharmaceutical composition comprises about 117,600 U.S.P. units lipase, about 490,000 U.S.P. units protease, and about 490,000 U.S.P. units amylase.

118. The method of claim 90, wherein the pharmaceutical composition comprises about 134,400 U.S.P. units lipase, about 560,000 U.S.P. units protease, and about 560,000 U.S.P. units amylase.

119. The method of claim 90, wherein the pharmaceutical composition comprises about 151,200 U.S.P. units lipase, about 630,000 U.S.P. units protease, and about 630,000 U.S.P. units amylase.

120. The method of claim 90, wherein the pharmaceutical composition comprises about 168,000 U.S.P. units lipase, about 700,000 U.S.P. units protease, and about 700,000 U.S.P. units amylase.

121. The method of claim 90, wherein the pharmaceutical composition comprises about 184,800 U.S.P. units lipase, about 770,000 U.S.P. units protease, and about 770,000 U.S.P. units amylase.

122. The method of claim 90, wherein the pharmaceutical composition comprises about 201,600 U.S.P. units lipase, about 840,000 U.S.P. units protease, and about 840,000 U.S.P. units amylase.

123. The method of claim 90, wherein the pharmaceutical composition comprises about 218,400 U.S.P. units lipase, about 910,000 U.S.P. units protease, and about 910,000 U.S.P. units amylase.

124. The method of claim 90, wherein the pharmaceutical composition comprises about 235,200 U.S.P. units lipase, about 980,000 U.S.P. units protease, and about 980,000 U.S.P. units amylase.

125. The method of claim 90, wherein the pharmaceutical composition comprises about 252,000 U.S.P. units lipase, about 1,050,000 U.S.P. units protease, and about 1,050,000 U.S.P. units amylase.

126. The method of any one of claims 110-125, further comprising a supplemental amount of protease selected from the group consisting of about 0 U.S.P. units; about 20,000 U.S.P. units; about 40,000 U.S.P. units; about 60,000 U.S.P. units; about 80,000 U.S.P. units; about 100,000 U.S.P. units; about 120,000 U.S.P. units; about 140,000 U.S.P. units; and about 160,000 U.S.P. units protease.
